(19) United States
(12) Patent Application Publication
(10) Pub. No.: US 2022/0033913 A1
(43) Pub. Date: Feb. 3, 2022

(54) GENOMIC REARRANGEMENTS ASSOCIATED WITH PROSTATE CANCER AND METHODS OF USING THE SAME

(71) Applicants: THE HENRY M. JACKSON FOUNDATION FOR THE ADVANCEMENT OF MILITARY MEDICINE, INC., BETHESDA, MD (US); CHILDREN'S MEDICAL CENTER CORPORATION, BOSTON, MA (US)

(72) Inventors: Albert DOBI, Rockville, MD (US); Gyorgy PETROVICS, Bethesda, MD (US); Shiv K. SRIVASTAVA, Potomac, MD (US); Hua LI, Bethesda, MD (US); Zoltan SZALLASI, Boston, MA (US)

(21) Appl. No.: 17/312,248

(22) PCT Filed: Dec. 12, 2019
(86) PCT No.: PCT/US2019/066094

§ 371 Date: Jun. 9, 2021

Related U.S. Application Data
(60) Provisional application No. 62/779,035, filed on Dec. 13, 2018.

(51) Int. Cl. C12Q 1/6886

(52) U.S. Cl. CPC C12Q 1/6886; C12Q 2600/158; C12Q 2600/156

(57) ABSTRACT

The present disclosure provides methods of identifying or characterizing prostate cancer comprising detecting in a biological sample the presence or absence of a genomic rearrangement that results in a deletion of an LSAMP gene and detecting in a biological sample the presence or absence of a genomic rearrangement that results in a deletion of a CHD1 gene. In certain embodiments, the patient self-identifies as being of African descent. Also disclosed herein are methods of testing for the presence of genomic rearrangements in an LSAMP gene and a CHD1 gene in a biological sample. The LSAMP and CHD1 genomic rearrangements serves as a biomarker for prostate cancer and can be used to stratify prostate cancer based on ethnicity or the severity or aggressiveness of prostate cancer and/or identify a patient for prostate cancer treatment. Also provided are kits for diagnosing and prognosing prostate cancer and methods of selecting a targeted prostate cancer treatment for a patient.

### CROSS-REFERENCE TO RELATED APPLICATIONS

This application claims the benefit of, and relies on the filing date of, U.S. provisional patent application No. 62/779,035, filed 13 Dec. 2018, the entire contents of which are incorporated herein by reference.

### GOVERNMENT INTEREST

This invention was made in part with Government support under grant HU0001-10-2-0002 awarded by Uniformed Services University. The Government has certain rights in the invention.

### SEQUENCE LISTING

This application contains a Sequence Listing which has been submitted electronically in ASCII format and is hereby incorporated by reference in its entirety. Said ASCII copy, created on 9 Dec. 2019, is named HMJ-163-PCT_SL.txt and is 208,585 bytes in size.

### FIELD

This application generally relates to gene profiles, methods of detecting the same and their use in diagnosing/prognosing and/or treating prostate cancer.

### BACKGROUND

Prostate cancer is the second leading cause of cancer death among men in the United States, with an anticipated 174,650 newly diagnosed cases and approximately 31,620 deaths in 2019 [1]. It is estimated that 1 in 6 men of African ancestry will be diagnosed with cancer of the prostate (CaP) in their lifetime, in comparison with 1 in 8 men of Caucasian ancestry.

Emerging data support biological and genetic differences between CaP patients of African descent (AD) and CaP patients of Caucasian descent (CD). Tumor sequencing studies have highlighted frequent alterations of ERG, PTEN, and SPOP genes in early stages of CaP, and of the androgen receptor (AR), p53, PIK3CB, and other genes in metastatic CaP or castration resistant prostate cancer. The majority of these studies were performed in men of European ancestry. ERG oncogenic fusion and PTEN deletion are found to be more frequent in CaP patients of CD than in CaP patients of AD, while recurrent deletions in LSAMP locus have been found to be more prevalent in CaP patients of AD than in CaP patients of CD [Petrovics et al., EBioMedicine 2015; 2:1957-64]. The lower frequency of the key biomarkers (ERG, PCA3) in other racial groups, including AD CaP patients, has recently been highlighted.

The racial disparity exists from presentation and diagnosis through treatment, survival, and quality of life [2]. Researchers have suggested that socio-economic status (SES) contributes significantly to these disparities including CaP-specific mortality [3]. As well, there is evidence that reduced access to care is associated with poor CaP outcomes, which is more prevalent among men of AD than men of CD [4].

However, there are populations in which men of AD have similar outcomes to men of CD. Sridhar and colleagues [5] published a meta-analysis in which they concluded that when SES is accounted for, there are no differences in the overall and CaP-specific survival between men of CD and AD. Similarly, the military and veteran populations (systems of equal access and screening) do not observe differences in survival across race [6], and differences in pathologic stage at diagnosis narrowed by the early 2000s in a veterans' cohort [7]. Of note, both of these studies showed that men of AD were more likely to have higher Gleason scores and prostate-specific antigen (PSA) levels than men of CD [6, 7].

While socio-economic factors may contribute to CaP outcomes, they do not seem to account for all variables associated with the diagnosis and disease risk. Several studies support that men of AD have a higher incidence of CaP compared to men of CD [1, 8, 9]. Studies also show that men of AD have a significantly higher PSA at diagnosis, higher grade disease on biopsy, greater tumor volume for each stage, and a shorter PSA doubling time before radical prostatectomy [10-12]. Biological differences between prostate cancers from men of CD and AD have been noted in the tumor microenvironment with regard to stress and inflammatory responses [13]. Although questions remain to be clarified over the role of biological differences, observed differences in incidence and disease aggressiveness at presentation indicate a potential role for different pathways of prostate carcinogenesis between men of AD and CD.

Over the past decade, much research has focused on alterations of cancer genes and their effects in CaP [14-16]. Variations in prevalence across ethnicity and race have been noted in the TMPRSS2:ERG gene fusion that is recurrent in CaP and is the most common known oncogene in CaP [17, 18]. Accumulating data suggest that there are differences of ERG oncogenic alterations across ethnicities [17, 19-21]. Significantly greater ERG expression in men of CD compared to men of AD was noted in initial papers describing ERG overexpression and ERG splice variants [17, 21]. The CD vs. AD difference is even more pronounced (50% versus 16%) between and in patients with high Gleason grade (8-10) tumors [37]. Thus, ERG is a major difference in somatic gene alteration between these ethnic groups. Yet beyond TMPRSS2:ERG, little is known regarding the genetic basis for the CaP, and the disparity between AA and CA men remains unknown [24].

Therefore, new biomarkers and therapeutic markers that are specific for distinct ethnic populations and provide more accurate diagnostic and/or prognostic potential are needed.

### SUMMARY

One aspect of the present disclosure is directed to methods of identifying or characterizing prostate cancer in a subject based on the detection of the presence or absence of a genomic rearrangement that results in an LSAMP gene deletion or detection of the presence or absence of a genomic rearrangement that results in a CHD1 gene deletion in a biological sample comprising prostate cells. The presence of either genomic rearrangement may identify prostate cancer in the subject or characterize the prostate cancer in the subject as being an aggressive form of prostate cancer or as having an increased risk of developing into an aggressive form of prostate cancer, particularly in human subjects of African descent. In certain embodiments, the presence of either or both genomic rearrangements may identify prostate cancer as having an increased risk of metastasizing. In certain embodiments, the presence of either or both genomic rearrangements may identify prostate cancer has having a combined Gleason score of 8-10 or an increased risk of developing into prostate cancer having a combined Gleason score of 8-10. In certain embodiments, the presence of either or both genomic rearrangements may identify a prostate cancer as having an increased risk of biochemical recurrence.

Another aspect of the present disclosure is directed to a method of testing for the presence of a genomic rearrangement in an LSAMP gene and a CHD1 gene comprising assaying the biological sample to determine if it contains either a genomic rearrangement that results in deletion of an LSAMP gene or a genomic rearrangement that results in deletion of a CHD1 gene. Another genomic rearrangement of interest that is associated with prostate cancer is the PTEN deletion. While the PTEN gene is a common tumor suppressor and its deletion is known to be associated with cancer, it has been surprisingly discovered that the PTEN deletion occurs with significantly different frequencies in different ethnic groups and is markedly absent in subjects of African descent. Understanding the stratification of cancer-related genomic rearrangements, such as the PTEN deletion, between different patient populations provides important information to instruct treatment options for prostate cancer patients.

In some embodiments, the genomic rearrangement that results in deletion of an LSAMP gene can further involve a ZBTB20 gene, such as a ZBTB20 gene deletion. In some embodiments, the biological sample comprises human prostate cells or nucleic acids isolated therefrom. In some embodiments the biological sample is a tissue sample, a cell sample, a blood sample, a serum sample, a semen or seminal fluid sample, or a urine sample. The genomic rearrangement that results in deletion of an LSAMP gene and the genomic rearrangement that results in deletion of a CHD1 gene can be measured at either the nucleic acid or protein level.

In some embodiments, the methods disclosed herein further comprise detecting the presence or absence of a genomic rearrangement that results in the deletion of a PTEN gene in the biological sample, and in some further embodiments, the methods further comprise detecting the presence or absence of a genomic rearrangement that results in a TMPRSS2:ERG gene fusion in the biological sample.

In some embodiments, the genomic rearrangement that results in the deletion of an LSAMP gene is a genomic rearrangement on chromosome region 3q13 between a ZBTB20 gene and and LSAMP gene, and in various embodiments, the genomic rearrangement that results in the deletion of an LSAMP gene is a deletion that spans the ZBTB20 gene and the LSAMP gene.

In some embodiments, the methods disclosed herein further comprise measuring the expression of one or more of the following genes: PTEN, COL10A1, HOXC4, ESPL1, MMP9, ABCA13, PCDHGA1, AGSK1, ERG, AMACR, PCA3, or KLK3. In certain embodiments, measuring the expression of a gene may indicate the presence of a genomic rearrangement that has resulted in the deletion of that gene, such as the deletion of the PTEN gene.

Given the prognostic value of the genomic rearrangement that results in deletion of an LSAMP gene or the genomic rearrangement that results in deletion of a CHD1 gene, the methods may further comprise a step of selecting a treatment regimen for the subject based on the detection of the presence of a genomic rearrangement, such as a genomic rearrangement resulting in the deletion of the LSAMP gene or the deletion of the CHD1 gene. In some embodiments, the methods may further comprise treating the subject with a treatment regimen if the presence of a genomic rearrangement, such as a genomic rearrangement resulting in the deletion of the LSAMP gene or the deletion of the CHD1 gene, is detected in the biological sample obtained from the subject. Alternatively, the methods may further comprise a step of increasing the frequency of monitoring the subject for the development of prostate cancer or a more aggressive form of prostate cancer. In some embodiments, the treatment regimen may comprise at least one of surgery, radiation therapy, hormone therapy, chemotherapy, biological therapy, or high intensity focused ultrasound. In certain embodiments, the treatment regimen may comprise at least one of poly(ADP-ribose) polymerase (PARP) inhibitors and platinum-based agents.

In some embodiments, the methods disclosed herein may further comprise a step of testing the biological sample from the subject to confirm that the biological sample does not contain a genomic rearrangement that results in deletion of a PTEN gene.

Another aspect of the present disclosure is directed to a kit for use in diagnosing or prognosing prostate cancer comprising a first oligonucleotide probe for detecting a genomic rearrangement that results in a deletion of an LSAMP gene and a second oligonucleotide probe for detecting a genomic rearrangement that results in a deletion of a CHD1 gene. In some embodiments, the kit is for use by human subjects of African descent. In other various embodiments, the kit contains oligonucleotide probes for detecting no more than 500 different genes, such as no more than 250, 100, 50, 25, 15, 10, 5, or 2 different genes.

The kits disclosed herein may further comprise an oligonucleotide probe for detecting a gene selected from PTEN, COL10A1, HOXC4, ESPL1, MMP9, ABCA13, PCDHGA1, AGSK1, ERG, AMACR, PCA3, and KLK3. The oligonucleotide probe is optionally labeled. In certain embodiments, the kits contain oligonucleotide probes for detecting no more than 500, 250, 100, 50, 25, 15, 10, 5, or 2 different genes. In yet another embodiment, the first and second oligonucleotide probes are attached to a surface of an array, and in certain embodiments, the array comprises no more than 500, 250, 100, 50, 25, 15, 10, or 5 different addressable elements. In certain embodiments, the first and second oligonucleotide probes are labeled. In certain embodiments, the kits disclosed herein further comprise an antibody probe for detecting an ERG oncoprotein.

Another aspect of the present disclosure is directed to a method of selecting a targeted prostate cancer treatment for a patient, such as a patient of African descent, wherein the method comprises (a) identifying a patient as having prostate cells that comprise at least one of a first genomic rearrangement that results in a deletion of an LSAMP gene and a second genomic rearrangement that results in a deletion of a CHD1 gene; (b) excluding prostate cancer therapy that targets the PI3K/PTEN/Akt/mTOR pathway as a treatment option for the patient; and selecting an appropriate prostate cancer treatment for the patient. In some embodiments, the method further comprises a step of testing a biological sample from the patient, wherein the biological sample comprises prostate cells to confirm that the prostate cells do not contain a genomic rearrangement that results in a deletion of a PTEN gene. In some embodiments, the appropriate cancer treatment comprises administration of at least one of PARP inhibitors and platinum-based agents.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced.

FIG. 1 is a map showing the presence or absence of genomic rearrangements resulting in a deletion of an LSAMP or a CHD1 gene in a cohort of 42 African-American patient specimens, as disclosed in the Example. The black circles indicate that the patient later developed a bone metastasis of the prostate cancer, whereas white circles indicate that the patient did not later develop a bone metastasis of the prostate cancer. The black rectangles indicate that the patient specimen was determined to contain a genomic rearrangement indicating a deletion in the LSAMP and/or the CHD1 gene. The white rectangles indicate that the patient specimen was determined not to contain a genomic rearrangement indicating a deletion in the LSAMP and/or the CHD1 gene.

FIG. 2 is a map showing the presence or absence of genomic rearrangements resulting in a deletion of an LSAMP or a CHD1 gene in a cohort of 59 Caucasian-American patient specimens, as disclosed in the Example. The black circles indicate that the patient later developed a bone metastasis of the prostate cancer, whereas white circles indicate that the patient did not later develop a biochemical recurrence of the prostate cancer. The black rectangles indicate that the patient specimen was determined to contain a genomic rearrangement indicating a deletion in the LSAMP and/or the CHD1 gene. The white rectangles indicate that the patient specimen was determined not to contain a genomic rearrangement indicating a deletion in the LSAMP and/or the CHD1 gene.

### DETAILED DESCRIPTION

The following detailed description is presented to enable any person skilled in the art to make and use the invention. For purposes of explanation, specific nomenclature is set forth to provide a thorough understanding of the present invention. However, it will be apparent to one skilled in the art that these specific details are not required to practice the invention. Descriptions of specific applications are provided only as representative examples. The present invention is not intended to be limited to the embodiments shown, but is to be accorded the widest possible scope consistent with the principles and features disclosed herein.

### Definitions

In order that the present invention may be more readily understood, certain terms are first defined. Additional definitions are set forth throughout the detailed description.

The term “of African descent” refers to individuals who self-identify as being of African descent, including individuals who self-identify as being African-American, and individuals determined to have genetic markers correlated with African ancestry, also called Ancestry Informative Markers (AIM), such as the AIMs identified in Judith Kidd et al., Analyses of a set of 128 ancestry informative single-nucleotide polymorphisms in a global set of 119 population samples, Investigative Genetics, (2):1, 2011, which reference is incorporated by reference in its entirety.

The term “of Caucasian descent” refers to individuals who self-identify as being of Caucasian descent, including individuals who self-identify as being Caucasian-American, and individuals determined to have genetic markers correlated with Caucasian (e.g., European or Asian (Western, Central or Southern) ancestry, also called Ancestry Informative Markers (AIM), such as the AIMs identified in Judith Kidd et al., Analyses of a set of 128 ancestry informative single-nucleotide polymorphisms in a global set of 119 population samples, Investigative Genetics, (2):1, 2011, which reference is incorporated by reference in its entirety.

The term “antibody” refers to an immunoglobulin or antigen-binding fragment thereof, and encompasses any polypeptide comprising an antigen-binding fragment or an antigen-binding domain. The term includes but is not limited to polyclonal, monoclonal, monospecific, polyspecific, humanized, human, single-chain, chimeric, synthetic, recombinant, hybrid, mutated, grafted, and in vitro generated antibodies. Unless preceded by the word “intact”, the term “antibody” includes antibody fragments such as Fab, F(ab′)2, Fv, scFv, Fd, dAb, and other antibody fragments that retain antigen-binding function. Unless otherwise specified, an antibody is not necessarily from any particular source, nor is it produced by any particular method.

The term “detecting” or “detection” means any of a variety of methods known in the art for determining the presence, absence, or amount of a nucleic acid or a protein. As used throughout the specification, the term “detecting” or “detection” includes either qualitative or quantitative detection.

The term “therapeutically effective amount” refers to a dosage or amount that is sufficient for treating an indicated disease or condition.

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The terms “polypeptide,” “peptide,” and “protein” are used interchangeably herein to refer to polymers of amino acids.

The term “polypeptide probe” as used herein refers to a labeled (e.g., fluorescently or isotopically labeled) polypeptide that can be used in a protein detection assay (e.g., mass spectrometry) to quantify a polypeptide of interest in a biological sample.

The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site.

A “variation” or “variant” refers to an allele sequence that is different from the reference at as little as a single base or for a longer interval.

The term “LSAMP gene deletion” or “deletion of an LSAMP gene” and the like refers to any deletion of the LSAMP gene that is associated with prostate cancer. LSAMP refers to limbic system associated membrane protein (LSAMP), which has been assigned the unique Huge Gene Nomenclature Committee (HGNC) identifier code HGNC:6705 and is located on the chromosome region 3q13.31.

The term “CHD1 gene deletion” or “deletion of a CHD1 gene” and the like refers to any deletion of the CHD1 gene that is associated with prostate cancer. CHD1 refers to chromodomain helicase DNA binding protein 1 (CHD1), which has been assigned the unique HGNC identifier code HGNC:1915 and is located on the chromosome region 5q15-q21.1.

The term “PTEN gene deletion” or “deletion of a PTEN gene” and the like refers to any deletion of the PTEN gene that is associated with prostate cancer. PTEN refers to phosphatase and tensin homolog (PTEN), which has been assigned the unique HGNC identifier code HGNC:9588 and is located on the chromosome region 10q23.31.

The term “ERG” or “ERG gene” refers to Ets-related gene (ERG), which has been assigned the unique HGNC identifier code: HGNC:3446, and includes ERG gene fusion products that are prevalent in prostate cancer, including TMPRSS2:ERG fusion products. Analyzing the expression of ERG or the ERG gene includes analyzing the expression of ERG gene protein (ERG oncoprotein) or mRNA products that are associated with prostate cancer, such as TMPRSS2:ERG.

As used herein, the term “aggressive form of prostate cancer” refers to prostate cancer with a primary Gleason grade of 4 or 5 (also known as “poorly differentiated” prostate cancer or prostate cancer that has metastasized or has recurred following prostatectomy) or a combined Gleason score of at least 6, such as a Gleason score of 6, 7, 8, 9, or 10.

As used herein, the term “Gleason 6-7” refers to Gleason grade 3+3 and 3+4. It is also referred to in the art as primary pattern 3 or primary Gleason pattern 3.

As used herein, a “biological sample” comprises human prostate cells or nucleic acids isolated therefrom. A biological sample of the present disclosure includes, but is not limited to a tissue sample, a cell sample, a blood sample, a serum sample, a semen or seminal fluid sample, a urine sample or any combination thereof.

As used herein, a “biochemical recurrence” (BCR) refers to a post-radical prostatectomy serum prostate-specific antigen (PSA) increase that indicates treatment by hormonal ablation and/or chemotherapy. The PSA increase is typically a PSA greater than or equal to 0.1 ng/mL, or a PSA greater than or equal to 0.2 ng/mL, measured no less than eight weeks after radical prostatectomy, followed by a successive, confirmatory PSA level greater than or equal to 0.2 ng/mL.

Detecting Gene Expression

As used herein, measuring or detecting the expression of any of the foregoing genes or nucleic acids comprises measuring or detecting any nucleic acid transcript (e.g., mRNA, cDNA, or genomic DNA) corresponding to the gene of interest or the protein encoded thereby. The presence or absence of a gene may be detected by measuring or detecting the expression of a gene or nucleic acids, for example if the gene or nucleic acids are not detected, or if the measurement of the expression of the gene or nucleic acid falls below a threshold level, the gene or nucleic acids may be determined to be absent. Likewise, if the gene or nucleic acids are detected, or if the measurement of the expression of the gene or nucleic acid falls above a threshold level, the gene or nucleic acids may be determined to be present. If a gene is associated with more than one mRNA transcript or isoform, the expression of the gene can be measured or detected by measuring or detecting one or more of the mRNA transcripts of the gene, or all of the mRNA transcripts associated with the gene.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, microarray analysis, RNA-sequencing, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual, 4th Ed., Cold Spring Harbor Press, Cold Spring Harbor, N.Y., 2012. Other techniques include digital, multiplexed analysis of gene expression, such as the nCounter® (NanoString Technologies, Seattle, Wash.) gene expression assays, which are further described in US20100112710 and US20100047924.

Detecting a nucleic acid of interest generally involves hybridization between a target (e.g., mRNA, cDNA, or genomic DNA) and a probe. The nucleic acid sequences of the genes described herein are known. Therefore, one of skill in the art can readily design hybridization probes for detecting those genes. See, e.g., Sambrook et al., Molecular Cloning: A Laboratory Manual, 4th Ed., Cold Spring Harbor Press, Cold Spring Harbor, N.Y., 2012. Each probe may be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, for example hybridization to the polyA tail would not provide specificity. If a target has multiple splice variants, it is possible to design a hybridization reagent that recognizes a region common to each variant and/or to use more than one reagent, each of which may recognize one or more variants.

In some embodiments, RNA-sequencing (RNA-seq) is used. As used herein, RNA-seq, also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample's RNA from a genome at a given moment in time. In certain embodiments, RNA is converted to cDNA fragments via reverse transcription prior to sequencing, and, in certain embodiments, RNA can be directly sequenced from RNA fragments without conversion to cDNA. Adaptors may be attached to the 5′ and/or 3′ ends of the fragments, and the RNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLiD system).

In some embodiments, microarray analysis or a PCR-based method is used, including, but not limited to, real-time PCR, nested PCT, quantitative PCR, multiplex PCR, and digital drop PCR. In this respect, measuring the expression of the foregoing nucleic acids in a biological sample can comprise, for instance, contacting a sample containing or suspected of containing prostate cancer cells or exosomes derived therefrom with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4th Ed., Cold Spring Harbor Press, Cold Spring Harbor, N.Y., 2012. Similarly, detailed protocols for preparing and using microarrays to analyze gene expression are known in the art and described herein.

Alternatively or additionally, expression levels of genes can be determined at the protein level, meaning that levels of proteins encoded by the genes discussed herein are measured. Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins. In certain embodiments, ERG oncoprotein can be detected by an antibody array, as described, for example, in the art. See, e.g., Furusato et al., ERG oncoprotein expression in prostate cancer: clonal progression of ERG-positive tumor cells and potential for ERG-based stratification, PROSTATE CANCER AND PROSTATIC DISEASE, 2010; 13:228-237.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g., PCR) and monitoring the amplified nucleic acid product.

Although immunoassays have been used for the identification and quantification of proteins, recent advances in mass spectrometry (MS) techniques have led to the development of sensitive, high-throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include, for example, two-dimensional electrophoresis, liquid chromatography, and capillary electrophoresis. Selected reaction monitoring (SRM), also known as multiple reaction monitoring (MRM), has also emerged as a useful high-throughput MS-based technique for quantifying targeted proteins in complex biological samples, including prostate cancer biomarkers that are encoded by gene fusions (e.g., TMPRSS2/ERG).

Genomic Rearrangements that Result in Deletion of LSAMP or CHD1

In certain embodiments, a genomic rearrangement resulting in the deletion of an LSAMP gene or a genomic rearrangement resulting in the deletion of a CHD1 gene may be used to identify or characterize prostate cancer in a human subject of African descent. Likewise, the absence of a genomic rearrangement resulting in the deletion of a PTEN gene, coupled with the presence of a genomic rearrangement resulting in the deletion of an LSAMP gene or a genomic rearrangement resulting in the deletion of a CHD1 gene may be used to identify or characterize prostate cancer in a human subject of African descent. In certain embodiments, the genomic rearrangement that results in the deletion of an LSAMP gene spans the ZBTB20 gene and the LSAMP gene.

The unique identifier code assigned by HGNC for the human LSAMP gene is HGNC:6705. The Entrez Gene code for LSAMP is 4045. The nucleotide and amino acid sequences of LSAMP are known and represented by the NCBI Reference Sequence NM_002338.3, GI:257467557 (SEQ ID NO:1 and SEQ ID NO:2). The chromosomal location of the LSAMP gene is 3q13.2-q21. The LSAMP gene encodes a neuronal surface glycoprotein found in cortical and subcortical regions of the limbic system. LSAMP has been reported as a tumor suppressor gene (Baroy et al., 2014, Mol Cancer 28; 13:93). For example, Kuhn et al. reported a recurrent deletion in chromosome region 3q13.31, which contains the LSAMP gene, in a subset of core binding factor acute myeloid leukemia [29]. In osteosarcoma, chromosome region 3q13.31 was identified as the most altered genomic region, with most alterations taking the form of a deletion, including, in certain instances, deletion of a region that contains the LSAMP gene [30]. A chromosomal translocation (t1;3) with a breakpoint involving the NORE1 gene of chromosome region 1q32.1 and the LSAMP gene of chromosome region 3q13.3 was identified in clear cell renal carcinomas [31]. A chromosomal translocation in epithelial ovarian carcinoma has also been identified [32]. Single nucleotide variations of LSAMP have been shown to be a significant predictor of prostate cancer-specific mortality [33].

The unique identified code assigned by HGNC for the human CHD1 gene is HGNC:1915. The Entrez Gene code for CHD1 is 1105. The nucleotide and amino acid sequences of CHD1 are known and represented by the NCBI Reference Sequence NM_001364113.1, GI:1396658733 (SEQ ID NO:3). The chromosomal location of the CHD1 gene is 5q15-q21.1. CHD1 is a known tumor suppressor gene whose deletion has been implication in CaP (Shenoy et al., 2017, Ann Oncol 28; 7:1495-1507), which reference is hereby incorporated by reference in its entirety. CHD1 is a DNA helicase and chromatin remodeler that functions in the DNA damage control mechanism and regulates chromatin assembly and transcription. Deletion of CHD1 may be associated with mutations in the SPOP gene, wherein the prostate cancer sample does not contain TMPRSS:ERG gene fusion and/or does not contain a PTEN deletion. Loss of CHD1 has been shown to increase the cancer cell sensitivity to DNA damage. The increased sensitivity results in an enhanced lethal response to therapies that inhibit the DNA damage control system (Shenoy et al., 2017). Homozygous deletion of the CHD1 gene has been found in 4.7% of primary prostate cancers and in 9% of metastatic castration resistant prostate cancers of Caucasian descents (The Cancer Genome Atlas Research Network, 2015 Cell 163(4):1011-1025; Robinson et al., 2015, Cell 161(5):1215-1228). Among African-American patients, significantly higher homozygous deletion frequency (28%) of CHD1 in primary prostate tumor samples has been observed, as well as rapid disease progression.

The unique identifier code assigned by HGNC for the ZBTB20 gene is HGNC:13503. The Entrez Gene code for ZBTB20 is 26137. ZBTB20 is a DNA binding protein and is believed to be a transcription factor. There are at least 7 alternative transcript variants. There are at least four distinct promoters that can initiate transcription from at least four distinct sites within the ZBTB20 locus, producing four variants of exon 1 of ZBTB20: E1, E1A, E1B, and E1C. Representative nucleotide and amino acid sequences of ZBTB20 variant 1 are known and represented by the NCBI Reference Sequence NM_001164342.1 GI:257900532 (SEQ ID NO:4 and SEQ ID NO:5). Variant 2 differs from variant 1 in the 5′ untranslated region, lacks a portion of the 5′ coding region, and initiates translation at a downstream start codon, compared to variant 1. The encoded isoform (2) has a shorter N-terminus compared to isoform 1. Variants 2-7 encode the same isoform (2). Representative nucleotide and amino acid sequences of ZBTB20 variant 2 are known and represented by the NCBI Reference Sequence NM_015642.4, GI:257900536 (SEQ ID NO:6 and SEQ ID NO:7). The chromosomal location of the ZBTB20 gene is 3q13.2.

In certain embodiments, a genomic rearrangement results in the deletion of an LSAMP gene or a genomic rearrangement results in the deletion of a CHD1 gene. Additional exemplary genomic rearrangements involving the LSAMP gene may be found, for example, in U.S. Published Patent Application No. 2016/0326595, which is hereby incorporated by reference in its entirety. In one embodiment, the genomic rearrangement comprises a gene fusion between the ZBTB20 gene and the LSAMP gene, such as a fusion between exon 1 (e.g., E1, E1A, E1B, or E1C) of the ZBTB20 gene and exon 4 of the LSAMP gene. In another embodiment, the genomic rearrangement comprises a gene inversion involving the ZBTB20 gene and the LSAMP gene. In another embodiments, the genomic rearrangement comprises a deletion in chromosome region 3q13, wherein the deletion spans both the ZBTB20 and LSAMP genes (or a portion of one or both genes). In yet another embodiment, the genomic rearrangement comprises a gene duplication involving the ZBTB20 and LSAMP genes.

Likewise, exemplary genomic rearrangements involving the CHD1 gene may include gene fusions, gene inversions, gene deletions, and gene duplications. In certain embodiments, the genomic rearrangement results in a deletion of the CHD1 gene, wherein all or a portion of the CHD1 gene is deleted.

Certain embodiments are directed to a method of collecting data for use in diagnosing or prognosing CaP, the method comprising assaying a biological sample comprising prostate cells (or nucleic acid or polypeptides isolated from prostate cells) to detect whether it contains a genomic rearrangement resulting in the deletion of the LSAMP gene and/or a genomic rearrangement resulting in the deletion of the CHD1 gene. The method may optionally include an additional step of diagnosing or prognosing CaP using the collected gene expression data. In one embodiment, detecting a genomic rearrangement resulting in either the deletion of the LSAMP gene or the deletion of the CHD1 gene indicates the presence of CaP in the biological sample or an increased likelihood of developing CaP. In another embodiment detecting a genomic rearrangement resulting in either the deletion of the LSAMP gene or the deletion of the CHD1 gene indicates the presence of an aggressive form of CaP in the biological sample or an increased likelihood of developing an aggressive form of CaP.

In some embodiments, the genomic rearrangement resulting in a deletion in the LSAMP gene comprises a deletion in chromosome region 3q13, wherein the deletion spans both the ZBTB20 and LSAMP genes (or a portion of one or both genes).

In certain embodiments, at least one of (1) a genomic rearrangement resulting in the deletion of an LSAMP gene, (2) a genomic rearrangement resulting in the deletion of a CHD1 gene, (3) a genomic rearrangement resulting in the deletion of a PTEN gene, or (4) over-expression of an ERG gene may be used to identify or characterize prostate cancer in a human subject regardless of race. In certain embodiments, a genomic rearrangement resulting in the deletion of any of LSAMP, CHD1, or PTEN or expression of ERG oncogene may indicate a prostate cancer having an increased risk of metastazing in a human subject regardless of race. In certain embodiments, a genomic rearrangement resulting in the deletion of any of LSAMP, CHD1, or PTEN or expression of ERG may indicate a prostate cancer as having a combined Gleason score of 8-10 or an increased risk of developing into a prostate cancer having a combined Gleason score of 8-10 in a human subject regardless of race, and in certain embodiments, a genomic rearrangement resulting in the deletion of any of LSAMP, CHD1, or PTEN or expression of ERG may indicate a prostate cancer as having an increased risk of biochemical recurrence in a human subject regardless of race.

The methods of collecting data or diagnosing and/or prognosing CaP may further comprise detecting expression of other genes associated with prostate cancer, including, but not limited to COL10A1, HOXC4, ESPL1, MMP9, ABCA13, PCDHGA1, and AGSK1. In another embodiment, the methods of collecting data or diagnosing and/or prognosing CaP may further comprise detecting expression of other genes associated with prostate cancer, including, but not limited to ERG, AMACR, KLK3, and PCA3. The unique identifier codes assigned by HGNC and Entrez Gene for these human genes that are more frequently overexpressed in patients of African descent and the accession number of representative sequences are provided in Table 1.

TABLE 1

| | HGNC | Entrez | | |
|---|---|---|---|---|
| Gene | ID | Gene ID | NCBI Reference | SEQ ID NOs. |
| COL10A1 | 2185 | 1300 | NM_000493.3 GI:98985802 | 8 |
| HOXC4 | 5126 | 3221 | NM_014620.5 GI:546232084 | 9 and 10 |
| ESPL1 | 16856 | 9700 | NM_012291.4 GI:134276942 | 11 and 12 |
| MMP9 | 7176 | 4318 | NM_004994.2 GI:74272286 | 13 and 14 |
| ABCA13 | 14638 | 154664 | AY204751.1 GI:30089663 | 15 and 16 |
| PCDHGA1 | 8696 | 56114 | NM_018912.2 GI:14196453 | 17 and 18 |
| AGSK1 | N/A | 80154 | NR_026811 GI:536293433 | 19 |
| | | | NR_033936.3 GI:536293365 | |
| | | | NR_103496.2 GI:536293435 | |
| ERG | | 2078 | NM_004449 | 20 |
| AMACR | | 23600 | NM_014324 | 21 |
| | | 114899 | | |
| | | 100534612 | | |
| KLK3 | | 354 | NM_001648 | 22 |
| PCA3 | | 50652 | NR_015342 | 23 |

Genomic Rearrangement that Results in Deletion of PTEN

PTEN (phosphatase and tensin homolog) is a known tumor suppressor gene that is mutated in a large number of cancers at high frequency. The protein encoded by this gene is a phosphatidylinositol-3,4,5-trisphosphate 3-phosphatase. It contains a tensin like domain as well as a catalytic domain similar to that of the dual specificity protein tyrosine phosphatases. Unlike most of the protein tyrosine phosphatases, PTEN preferentially dephosphorylates phosphoinositide substrates. It negatively regulates intracellular levels of phosphatidylinositol-3,4,5-trisphosphate in cells and functions as a tumor suppressor by negatively regulating AKT/PKB signaling pathway. Activation of growth factor receptors by binding of a growth factor to its receptor or by mutation of the growth factor receptor leads to activation of the PI3K/PTEN/Akt/mTOR cascade, which, among other things, leads to the activation of certain transcription factors [28]. PTEN normally acts to down regulate this pathway. Thus, in cancers that contain a PTEN gene deletion, the expression of the Akt gene and activation of mTOR is frequently increased.

The unique identifier codes assigned by HGNC and Entrez Gene for the human PTEN gene are HGNC:9588 and Entrez Gene:5728, respectively. The accession number of representative PTEN nucleic acid and polypeptide sequences is NM_000314.4, GI:257467557 (SEQ ID NO:24 and SEQ ID NO:25). The chromosomal location of the PTEN gene is 10q23.

Whole genome sequence analysis of prostate cancer samples from patients of of African descent and Caucasian descent disclosed a significant disparity between the genomic rearrangement of the PTEN locus in the different ethnic groups. More specifically, PTEN deletion was detected primarily in patients of Caucasian descent. Additional FISH analysis in a tissue microarray confirmed that PTEN deletion is an infrequent event in the development of prostate cancer in patients of African descent as compared to patients of Caucasian descent.

Accordingly, one aspect is directed to using this discovery about the disparity in the PTEN deletion across ethnic groups to make informed decisions about treatment options available to a subject who has prostate cancer. In particular, given the disclosed disparity in the PTEN deletion in prostate cancer from patients of Caucasian and African descent, as a general rule, prostate cancer therapies that target the PI3K/PTEN/Akt/mTOR pathway [28] should not be selected for patients of African descent. Or, at a minimum, a prostate cancer therapy that targets the PI3K/PTEN/Akt/mTOR pathway [28] should not be considered for a patient of African descent unless it is first confirmed by genetic testing that prostate cells from the patient contain the PTEN deletion. As such, one embodiment is directed to a method of selecting a targeted prostate cancer treatment for a patient of African descent, wherein the method comprises excluding a prostate cancer therapy that targets the PI3K/PTEN/Akt/mTOR pathway [28] as a treatment option; and selecting an appropriate prostate cancer treatment. In one embodiment, the method further comprises a step of testing a biological sample from the patient, wherein the biological sample comprises prostate cells to confirm that the prostate cells to do not contain a PTEN gene deletion.

There are various inhibitors that target the PI3K/PTEN/Akt/mTOR pathway, including PI3K inhibitors, Akt inhibitors, mTOR inhibitors, and dual PI3K/mTOR inhibitors. PI3K inhibitors include, but are not limited to LY-294002, wortmannin, PX-866, GDC-0941, CAL-10, XL-147, XL-756, IC87114, NVP-BKM120, and NVP-BYL719. Akt inhibitors include, but are not limited to, A-443654, GSK690693, VQD-002 (a.k.a. API-2, triciribine), KP372-1, KRX-0401 (perifosine), MK-2206, GSK2141795, LY317615 (enzasturin), erucylphosphocholine (ErPC), erucylphosphohomocholine (ErPC3), PBI-05204, RX-0201, and XL-418. mTOR inhibitors include, but are not limited to, rapamycin, modified rapamycins (rapalogs, e.g., CCI-779, afinitor, torisel, temsirolimus), AP-23573 (ridaforolimus), and RAD001 (afinitor, everolimus), metformin, OSI-027, PP-242, AZD8055, AZD2014, palomid 529, WAY600, WYE353, WYE687, WYE132, Ku0063794, and OXA-01. Dual PI3K/mTOR inhibitors include, but are not limited to, PI-103, NVP-BEZ235, PKI-587, PKI-402, PF-04691502, XL765, GNE-477, GSK2126458, and WJD008.

Detecting Genomic Rearrangements that Result in Gene Deletion

Measuring or detecting the expression of a genomic rearrangement of a gene, such as the LSAMP or CHD1 genes, in the methods described herein comprises measuring or detecting any nucleic acid transcript (e.g., mRNA, cDNA, or genomic DNA) that evidences the genomic rearrangement or any protein encoded by such a nucleic acid transcript, if applicable. Thus, in one embodiment, the genomic rearrangement results in the deletion of the LSAMP gene or the CHD1 gene. In one embodiment, a genomic rearrangement results in the deletion of the LSAMP gene, and a genomic rearrangement results in the deletion of the CHD1 gene. In one embodiment, detecting the presence of a genomic rearrangement that results in deletion of the LSAMP gene in the biological sample comprises detecting a deletion in chromosome region 3q13, and in one embodiment, the deletion spans the LSAMP gene or a portion thereof, while in another embodiment the deletion spans the ZBTB20 and LSAMP genes. In other embodiments, detecting the presence of the genomic rearrangement that results in deletion of the CHD1 gene in the biological sample comprises detecting a deletion in chromosome region 5q15-q21.1, wherein the deletion spans the CHD1 gene or a portion thereof.

The deletion of the LSAMP gene can be measured or detected by measuring or detecting one or more of the genomic sequences or mRNA/cDNA transcripts corresponding to the LSAMP deletion, or to all of the genomic sequences or mRNA/cDNA transcripts associated with the LSAMP gene.

The deletion of the CHD1 gene can be measured or detected by measuring or detecting one or more of the genomic sequences or mRNA/cDNA transcripts corresponding to the CHD1 deletion, or to all of the genomic sequences or mRNA/cDNA transcripts associated with the CHD1 gene.

Detecting a genomic rearrangement resulting in the deletion of the PTEN gene comprises detecting a deletion in chromosome region 10q23, wherein the deletion spans the PTEN gene or a portion thereof. The deletion of the PTEN gene can be measured or detected by measuring or detecting one or more of the genomic sequences or mRNA/cDNA transcripts corresponding to the PTEN deletion, or to all of the genomic sequences or mRNA/cDNA transcripts associated with the PTEN gene.

Chromosomal rearrangements can be detected by any method known in the art, including but not limited to DNA-sequencing (DNA-seq) and fluorescent in situ hybridization (FISH) analysis. For example, FISH analysis can be used to detect chromosomal rearrangements. In these embodiments, nucleic acid probes that hybridize under conditions of high stringency to the chromosomal rearrangement, such as deletion of the LSAMP gene, deletion of the CHD1 gene, or deletion of the PTEN gene, are incubated with a biological sample comprising prostate cells (or nucleic acid obtained therefrom). Other known in situ hybridization techniques can be used to detect chromosomal rearrangements, such as gene deletions. The nucleic acid probes (DNA or RNA) can hybridize to DNA or mRNA and can be designed to detect genomic rearrangements in the LSAMP, CHD1, or PTEN genes, including deletions. Typically, the nucleic acid probes are labeled to assist with detection of hybridization to a target sequence. Such labeled nucleic acid probes do not occur naturally. As used herein, DNA-seq refers to any high-throughput sequencing technique used to detect the presence and quantity of DNA in a sample. DNA-seq can be used to identify genomic variants and rearrangements, including, for example, gene fusions, gene deletions, gene inversions, and gene duplications. For example, in some embodiments, high-throughout sequencing techniques may be used to sequence relatively short fragments of sample DNA, which may then be mapped to a reference genome to identify genomic rearrangements. In certain embodiments, the genomic rearrangements may further include gene fusion events, amplifications, deletions, or mutations.

As discussed above, gene expression can be detected or measured on the basis of mRNA, cDNA, or protein levels, using any known quantitative or qualitative method, including, but not limited to, Northern Blotting, RNAse protection assays, microarray analysis, RNA-seq, or a nucleic acid amplification procedure, such as reverse-transcription PCR (RT-PCR) or real-time RT-PCR, also known as quantitative RT-PCR (qRT-PCR).

Detecting a nucleic acid of interest generally involves hybridization between a target (e.g., mRNA, cDNA, or genomic DNA) and a probe. One of skill in the art can readily design hybridization probes for detecting the genomic rearrangement of the genes, including deletion of the genes such as deletion of the LSAMP gene, the CHD1 gene, or the PTEN gene. See e.g., Sambrook et al., Molecular Cloning: A Laboratory Manual, 4th Ed., Cold Spring Harbor Press, Cold Spring Harbor, N.Y., 2012. Each probe should be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, e.g., hybridization to the polyA tail would not provide specificity. If a target has multiple splice variants, it is possible to design a hybridization reagent that recognizes a region common to each variant and/or to use more than one reagent, each of which may recognize one or more variants.

Stringency of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In certain embodiments, microarray analysis or a PCR-based method is used. In this respect, measuring the expression of the genomic rearrangement resulting in the deletion of the LSAMP gene or genomic rearrangement resulting in the deletion of the CHD1 gene in prostate cancer cells can comprise, for instance, contacting a sample containing or suspected of containing prostate cancer cells with polynucleotide probes specific to the LSAMP or CHD1 genomic rearrangement, or with primers designed to amplify a portion of the LSAMP or CHD1 deletion, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4th Ed., Cold Spring Harbor Press, Cold Spring Harbor, N.Y., 2012. Similarly, detailed protocols for preparing and using microarrays to analyze gene expression are known in the art and described herein. As one of ordinary skill in the art would appreciate, similar methods may be used to measure the expression of a genomic rearrangement resulting in the deletion of various other genes, including, for example, PTEN.

Alternatively or additionally, expression levels of various genomic rearrangements can be determined at the protein level, meaning that when the genomic rearrangement results in a truncated protein, such as a truncated LSAMP or CHD1 protein, the levels of such proteins encoded by the LSAMP or CHD1 genomic rearrangement are measured. Several methods and devices are well known for determining levels of proteins including immunoassays such as described in e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays typically include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO/2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with a sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g., PCR) and monitoring the amplified nucleic acid product.

Although immunoassays have typically been used for the identification and quantification of proteins, recent advances in mass spectrometry (MS) techniques have led to the development of sensitive, high throughput MS protein analyses. The MS methods can be used to detect low concentrations of proteins in complex biological samples. For example, it is possible to perform targeted MS by fractionating the biological sample prior to MS analysis. Common techniques for carrying out such fractionation prior to MS analysis include two-dimensional electrophoresis, liquid chromatography, and capillary electrophoresis [25], which reference is hereby incorporated by reference in its entirety. Selected reaction monitoring (SRM), also known as multiple reaction monitoring (MRM), has also emerged as a useful high throughput MS-based technique for quantifying targeted proteins in complex biological samples, including prostate cancer biomarkers that are encoded by gene fusions (e.g., TMPRSS2/ERG) [26, 27], which references are hereby incorporated by reference in their entirety.

Samples

The methods described in this application involve analysis of a genomic rearrangement resulting in the deletion of the LSAMP gene and/or a genomic rearrangement resulting in the deletion of the CHD1 gene in cells, including prostate cells. These prostate cells are found in a biological sample, such as, but not limited to, prostate tissue, blood, serum, plasma, urine, saliva, semen, seminal fluid, or prostatic fluid. Nucleic acids or polypeptides may be isolated from the cells prior to detecting gene expression.

In some embodiments, the biological sample comprises prostate tissue and is obtained through a biopsy, such as a transrectal or transperineal biopsy. In other embodiments, the biological sample is urine. Urine samples may be collected following a digital rectal examination (DRE) or a prostate biopsy. In other embodiments, the sample is blood, serum, or plasma, and contains circulating tumor cells that have detached from a primary tumor. The sample may also contain tumor-derived exosomes. Exosomes are small (typically 30 to 100 nm) membrane-bound particles that are released from normal, diseased, and neoplastic cells and are present in blood and other bodily fluids. The methods disclosed in this application can be used with samples collected from a variety of mammals, but preferably with samples obtained from a human subject.

Prostate Cancer

This application discloses certain genomic rearrangements that are associated with prostate cancer, wherein the genomic rearrangements result from the deletion of LSAMP or CHD1 genes. Detecting a genomic rearrangement resulting from the deletion of LSAMP or CHD1 genes in a biological sample can be used to identify cancer cells, such as prostate cancer cells, in a sample or to measure the severity or aggressiveness of prostate cancer, for example, distinguishing between well-differentiated prostate cancer and poorly-differentiated prostate cancer and/or identifying prostate cancer that has metastasized or recurred following prostatectomy or is more likely to metastasize or recur following prostatectomy.

When prostate cancer is found in a biopsy, it is typically graded to estimate how quickly it is likely to grow and spread. The most commonly used prostate cancer grading system, called Gleason grading, evaluates prostate cancer cells on a scale of 1 to 5, based on their pattern when viewed under a microscope.

Cancer cells that still resemble healthy prostate cells have uniform patterns with well-defined boundaries and are considered well differentiated (Gleason grades 1 and 2). The more closely the cancer cells resemble prostate tissue, the more the cells will behave like normal prostate tissue and the less aggressive the cancer. Gleason grade 3, the most common grade, shows cells that are moderately differentiated, that is, still somewhat well-differentiated, but with boundaries that are not as well-defined. Poorly-differentiated cancer cells have random patterns with poorly defined boundaries and no longer resemble prostate tissue (Gleason grades 4 and 5), indicating a more aggressive cancer.

Prostate cancers often have areas with different grades. A combined Gleason score is determined by adding the grades from the two most common cancer cell patterns within the tumor. For example, if the most common pattern is grade 4 and the second most common pattern is grade 3, then the combined Gleason score is 4+3=7. If there is only one pattern within the tumor, the combined Gleason score can be as low as 1+1=2 or as high as 5+5=10. Combined scores of 2 to 4 are considered well-differentiated, scores of 5 to 6 are considered moderately-differentiated and scores of 7 to 10 are considered poorly-differentiated. Cancers with a high Gleason score are more likely to have already spread beyond the prostate gland (metastasized) at the time they were found.

In general, the lower the Gleason score, the less aggressive the cancer and the better the prognosis (outlook for cure or long-term survival). The higher the Gleason score, the more aggressive the cancer and the poorer the prognosis for long-term, metastasis-free survival.

In certain embodiments, genomic rearrangements resulting in the deletion of the LSAMP gene and/or genomic rearrangements resulting in the deletion of the CHD1 gene indicate a prostate cancer having a high Gleason score, such as a combined Gleason score of 8-10, particularly in human subjects of African descent. In certain embodiments, genomic rearrangements resulting in the deletion of the LSAMP gene and/or genomic rearrangements resulting in the deletion of the CHD1 gene indicate a prostate cancer having an increased risk of developing into a prostate cancer having a high Gleason score, such as a combined Gleason score of 8-10, particularly in human subjects of African descent. In further embodiments, genomic rearrangements resulting in the deletion of the LSAMP gene and/or genomic rearrangements resulting in the deletion of the CHD1 gene indicate a prostate cancer having an increased risk of metastasizing, particularly in human subjects of African descent.

This application also discloses that genomic rearrangements resulting in the deletion of the tumor suppressor gene, PTEN, occur predominately, if not exclusively, in subjects of Caucasian descent. Conversely, the PTEN gene deletion is an infrequent event in prostate cancer from subjects of African descent, particularly in Gleason 6-7 prostate cancer from subjects of African descent. Of note, Gleason 6-7 (also called primary pattern 3) prostate cancer represents the most commonly diagnosed form of prostate cancer in the PSA screened patient population. For example, as disclosed herein, in a sample of subjects of African descent, all of whom exhibited a future biochemical recurrence event, the PTEN gene was deleted in about 21% of the samples, whereas in samples from subjects of Caucasian descent, the PTEN gene was deleted in about 77% of the samples.

Patient Treatment

This application describes methods of diagnosing and prognosing prostate cancer in a sample obtained from a subject, in which gene expression in prostate cells and/or tissues are analyzed. If a sample shows expression of a genomic rearrangement resulting in the deletion of the LSAMP gene and/or a genomic rearrangement resulting in the deletion of the CHD1 gene, then there is an increased likelihood that the subject has prostate cancer or a more advanced/aggressive form (e.g., poorly-differentiated prostate cancer) of prostate cancer if the subject is of African descent. In the event of such a result, the methods of detecting or prognosing prostate cancer may include one or more of the following steps: informing the patient that they are likely to have prostate cancer or poorly-differentiated prostate cancer; performing confirmatory histological examination of prostate tissue; increasing the frequency of monitoring the subject for the development of prostate cancer or a more aggressive form of prostate cancer; and/or treating the subject.

Thus, in certain aspects, if the detection step indicates that prostate cells from the subject have a genomic rearrangement resulting in the deletion of the LSAMP gene and/or the CHD1 gene, the methods further comprise a step of taking a prostate biopsy from the subject and examining the prostate tissue in the biopsy (e.g., histological examination) to confirm whether the patient has prostate cancer or an aggressive form of prostate cancer. Alternatively, the methods of detecting or prognosing prostate cancer may be used to assess the need for therapy or to monitor a response to a therapy (e.g., disease-free recurrence following surgery or other therapy), and, thus may include an additional step of treating a subject having prostate cancer.

Also provided herein are methods of treating prostate cancer in a patient of African descent, the method comprising administering a prostate cancer treatment regimen to the patient, wherein prior to the administering step, the patient has been identified as having prostate cancer or a more advanced/aggressive form (e.g., poorly-differentiated prostate cancer) of prostate cancer because a biological sample from the patient was tested and found to contain a genomic rearrangement resulting in the deletion of the LSAMP gene or a genomic rearrangement resulting in the deletion of the CHD1 gene. As discussed above, deletion of the CHD1 gene may increase cancer cell sensitivity to DNA damage, resulting in an enhanced lethal response to therapies that inhibit the DNA damage control system. Such DNA damage control system therapies may include, for example, radiation, PARP inhibitors, and platinum-based therapeutics, as discussed below. Therefore, in certain embodiments, the methods disclosed herein may stratify patients, such as patients of African descent, by CHD1 and/or LSAMP deletion status for DNA damage control system therapies.

Prostate cancer treatment options include, but are not limited to, surgery, radiation therapy, hormone therapy, chemotherapy, biological therapy, or high intensity focused ultrasound. Drugs for prostate cancer treatment include, but are not limited to: Abiraterone Acetate, Cabazitaxel, Degarelix, Enzalutamide (XTANDI), Jevtana (Cabazitaxel), Prednisone, Provenge (Sipuleucel-T), Sipuleucel-T, or Docetaxel.

Additional drugs that may be used to treat prostate cancer include poly(ADP ribose) polymerase (PARP) inhibitors and platinum-based agents. PARP inhibitors may include, for example, olaparib, rucaparib, and niraparib. PARP1 is a protein that functions to repair single-stranded nicks in DNA. Drugs that inhibit PARP1 (PARP inhibitors) result in DNA containing multiple double stranded breaks during replication, which can lead to cell death. Platinum-based agents are chemical complexes comprising platinum and cause crosslinking of DNA. Crosslinked DNA inhibits DNA repair and synthesis in cancerous cells. Exemplary platinum-based agents may include cisplatin, oxaliplatin, and carboplatin.

A method as described in this application may, after a positive result, include a further therapy step, e.g., surgery, radiation therapy, hormone therapy, chemotherapy, biological therapy, or high intensity focused ultrasound. In certain embodiments, the therapy step comprises administering a DNA damage control system therapy, such as radiation, a PARP inhibitor, or a platinum-based agent.

Compositions and Kits

The polynucleotide probes and/or primers or antibodies or polypeptide probes that are used in the methods described in this application can be arranged in a composition or a kit. Thus, some embodiments are directed to a composition, or compositions, for diagnosing or prognosing prostate cancer comprising a polynucleotide probe for detecting a first genomic rearrangement resulting in the deletion of an LSAMP gene and a polynucleotide probe for detecting a second genomic rearrangement resulting in the deletion of a CHD1 gene. All of the polynucleotide probes described herein may be optionally labeled. Such labeled polynucleotide probes are not naturally occurring.

In some embodiments, a composition for diagnosing or prognosing prostate cancer comprises a polynucleotide probe, wherein the polynucleotide probe is designed to detect a deletion in chromosome region 3q13, wherein the deletion spans the LSAMP gene or a portion thereof or spans the ZBTB20 and LSAMP genes. In other embodiments, a composition for diagnosing or prognosing prostate cancer comprises a polynucleotide probe designed to detect a deletion in chromosome region 5q15-21, wherein the depletion spans the CHD1 gene or a portion thereof. In still other embodiments, a composition for diagnosing or prognosing prostate cancer comprises a polynucleotide probe, wherein the polynucleotide probe is designed to detect a genomic rearrangement resulting in the deletion of a PTEN gene. These compositions may be combined into kits as discussed below.

The compositions for diagnosing or prognosing prostate cancer may also comprise primers. In some embodiments, a composition for diagnosing or prognosing prostate cancer comprises primers for amplifying a chimeric junction created by a deletion of the LSAMP gene in chromosome region 3q13, wherein the deletion spans the LSAMP gene or a portion thereof or spans the ZBTB20 and LSAMP genes. In some embodiments, a composition for diagnosing or prognosing prostate cancer comprises primers for amplifying a chimeric junction created by a deletion of the CHD1 gene in chromosome region 5q15-21, wherein the deletion spans the CHD1 gene or a portion thereof. In other embodiments, a composition for diagnosing or prognosing prostate cancer comprises primers for amplifying a chimeric junction created by a deletion of the PTEN gene. These compositions may be combined into kits as discussed below.

Typically for each gene deletion of interest (LSAMP, CHD1, and PTEN), a composition comprises a first polynucleotide primer comprising a sequence that hybridizes under high stringency conditions to a first nucleic acid that borders a 5′ end of the gene deletion; and a second polynucleotide primer comprising a sequence that hybridizes under high stringency conditions to the second nucleic acid that borders a 3′ end of the gene deletion, wherein the first and second polynucleotide primers are capable of amplifying a nucleotide sequence that spans the chimeric junction created by the gene deletion.

Another aspect of the present application is directed to kits for diagnosing or prognosing prostate cancer. In some embodiments, a kit for diagnosing or prognosing prostate cancer comprises a first composition comprising one or more polynucleotide probes and/or primers for detecting a genomic rearrangement resulting from a deletion of the LSAMP gene and a second composition comprising one or more polynucleotide probes and/or primers for detecting a genomic rearrangement resulting from a deletion of the CHD1 gene, as discussed above. In some embodiments, a kit comprises one or more polynucleotide probes and/or primers for detecting a deletion in chromosome region 3q13, wherein the deletion spans the ZBTB20 and LSAMP genes. In other embodiments, the kit further comprises one or more polynucleotide probes and/or primers for detecting a genomic rearrangement resulting from a deletion of the PTEN gene. In other embodiments, in addition to one or or more polynucleotide probes and/or primers for detecting a genomic rearrangement resulting from a deletion of the LSMAP, CHD1, and PTEN genes, the kit further comprises one or more polynucleotide probes and/or primers for detecting expression of the ERG gene and/or one or more antibody probes for detecting expression of the ERG oncoprotein.

In certain embodiments, a kit further comprises a composition comprising a polynucleotide probe that hybridizes under high stringency conditions to a gene selected from COL10A1, HOXC4, ESPL1, MMP9, ABCA13, PCDHGA1, and AGSK1. In other embodiments, a kit for diagnosing or prognosing prostate cancer further comprises a composition comprising a polynucleotide probe that hybridizes under high stringency conditions to a gene selected from ERG, AMACR, PCA3, and KLK3.

A kit for diagnosing or prognosing prostate cancer may also comprise antibodies. Thus, in some embodiments, a kit for diagnosing or prognosing prostate cancer comprises an antibody that binds to a polypeptide encoded by a genomic rearrangement resulting from a deletion of the LSAMP gene. In some embodiments, a kit comprises an antibody that binds to a polypeptide encoded by a genomic rearrangement resulting from a deletion of the CHD1 gene. In some embodiments, a kit comprises an antibody that binds to a polypeptide encoded by a genomic rearrangement resulting from a deletion of the PTEN gene. In some embodiments, a kit comprises an antibody that binds to a polypeptide encoded by the ERG gene. An antibody may be optionally labeled. In other embodiments, a kit further comprises one or more antibodies for detecting at least 1, 2, 3, 4, 5, 6, or 7 of the polypeptides encoded by following human genes: COL10A1, HOXC4, ESPL1, MMP9, ABCA13, PCDHGA1, and AGSK1. In other embodiments, a kit further comprises one or more antibodies for detecting ERG, AMACR, PCA3, or KLK3.

In some embodiments, a kit for diagnosing or prognosing prostate cancer includes instructional materials disclosing methods of use of the kit contents in a disclosed method. The instructional materials may be provided in any number of forms, including, but not limited to, written form (e.g., hardcopy paper, etc.), in an electronic form (e.g., solid state media or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes (e.g., polymerase), labeling compounds, and the like. Such kits and appropriate contents are well known to those of skill in the art. A kit can also include a reference or control sample or one or more polynucleotide probes for detecting expression of a control gene. A reference or control sample can be a biological sample or a data base.

Polynucleotide probes and antibodies described in this application are optionally labeled with a detectable label. Any detectable label used in conjunction with probe or antibody technology, as known by one of ordinary skill in the art, can be used. Such labeled probes or antibodies do not exist in nature. In a particular embodiment, the probe is labeled with a detectable label selected from the group consisting of: a fluorescent label, a chemiluminescent label, a quencher, a radioactive label, biotin, mass tags and/or gold.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Example

High quality genome sequence data and coverage obtained from histologically defined and precisely dissected primary CaP specimens was compared between cohorts of 59 patients of Caucasian descent and 42 patients of African descent (101 samples total) to evaluate the observed disparities of CaP incidence and mortality between the two ethnic groups. These data and analyses provide an evaluation of prostate cancer genomes from CaP patients of African descent (“AD”) and Caucasian descent (“CD”).

Materials and Methods: Validation of LSAMP and CHD1 Deletion Frequencies by Interphase FISH Assay

Fluorescence In Situ Hybridization (FISH) analysis for the detection of deletions at the ZBTB20-LSAMP and CHD1 locus was performed on whole-mounted sections and on prostate tumor tissue microarrays (TMAs) constructed from a cohort including 59 patients of Caucasian descent (CD) and 42 patients of African descent (AD) prostate cancer patients with radical prostatectomy specimens as described in Merseburger et al., Limitations of tissue microarrays in the evaluation of focal alterations of blc-2 and p53 in whole mounted derived prostate tissues, Oncol. Rep. (2003), 10: 223-228. Both the LSAMP and CHD1 FISH probes were obtained from CytoTest Inc. (Rockville, Md., USA). A ZBTB20-LSAMP locus-specific probe was constructed from bacterial artificial chromosome clones obtained from a commercial vendor, Life Technologies (Carlsbad, Calif., USA). Clones were cultured in Luria-Bertani (LB) medium prior to DNA isolation using standard procedures and labeling with CytoOrange fluorescent dye.

Clone combinations were selected in the core deleted region and tested in an iterative trial-and-error process to optimize signal intensity and specificity, resulting in a probe matching about 500 kbp of genomic sequence between the ZBTB20 and LSAMP loci, including the complete GAP43 gene. A second, LSAMP-centered probe was designed using the same process, resulting in a probe containing about 600 kbp of genomic sequence centered on and covering the entire LSAMP. A probe derived from chromosome 3-specific alpha satellite centromeric DNA, labeled with CytoGreen fluorescent dye, was used as a control. A CHD1 locus-specific probe (LSP) covers a chromosomal region which includes the entire CHD1 gene located on chromosome band 5q15-21. A chromosome specific probe D5S23, D5S721 covers the chromosomal region between the STS marker D5S23 and D5S721 and the region upstream and downstream of the two markers. Before use on tissue samples, locus-specific and control probes were mapped to normal human peripheral blood lymphocyte metaphases to confirm location and performance in interphase nuclei.

Whole-mounted prostate sections were pre-warmed in oven at 180° C. for one hour. Then, the sections were de-paraffinized with xylene for 30 min. The deparaffinized sections were dehydrated with 100% ethanol for 2 min for 3 times. The air-dried sections were immersed with in 100 mM Trizma Base+50 mM EDTA (pH 7.0) at 94° C. for 30 min. The sections were rinsed with Phosphate-Buffered Saline (PBS) solution (pH 7.0) for 5 min. The air-dried sections were digested with Digest All III (Invitrogen, Cat number: 00-3009) at 37° C. for 18 min. The sections were rinsed with PBS solution (pH 7.0) for 5 min. The sections were dehydrated with serial dilutions of ethanol (70%, 80%, 95% and 100%) for 2 min for each ethanol solution. The air-dried sections were combined with FISH probes (10 Cytotest LSP CHD1, CytoOrange/CCP 5, CytoGreen Cocktail, or Cytotest LSP LSAMP CytoOrange/CCP3 CytoGreen Cocktail). The sections applied in the FISH assay were covered with glass cover slips and sealed with rubber cement. The sealed sections were denatured at 94° C. on a hot plate for 10 min. Then, the sections were incubated at 37° C. overnight and then washed with the 2× Saline Sodium Solution (SSC) (pH 7.0) at 73° C. for 5 min. The sections were washed with 0.5×SSC (pH 7.0) at room temperature for 5 min for 3 times and rinsed with deionized water for 1 min. Finally, the air-dried sections were covered with DAPI Mount (ProLong Gold antifade reagent with DAPI, Invitrogen, Cat Number: P36935).

The FISH probe signals were observed under fluorescence microscope with 60× magnification objective. The excitation peaks of CytoOrange and CytoGreen labels were 551 and 495 nm, respectively. Tumor cells with at least two centromeres were counted. Numbers of centromeres and LSAMP/CHD1 signals were compared to determine whether cells were homozygous or heterozygous for this locus. A minimum of 100 cells from each tissue core were evaluated. Deletions were called when more than 75% of evaluable tumor cells showed loss of allele. Focal deletions were called when more than 25% of evaluable tumor cells showed loss of allele or when more than 50% evaluable tumor cells in each gland of a cluster of two or three tumor glands showed loss of allele. Benign prostatic glands and stroma served as built-in controls. Further, the protein expression of ERG was assessed with immunohistochemical staining.

Results

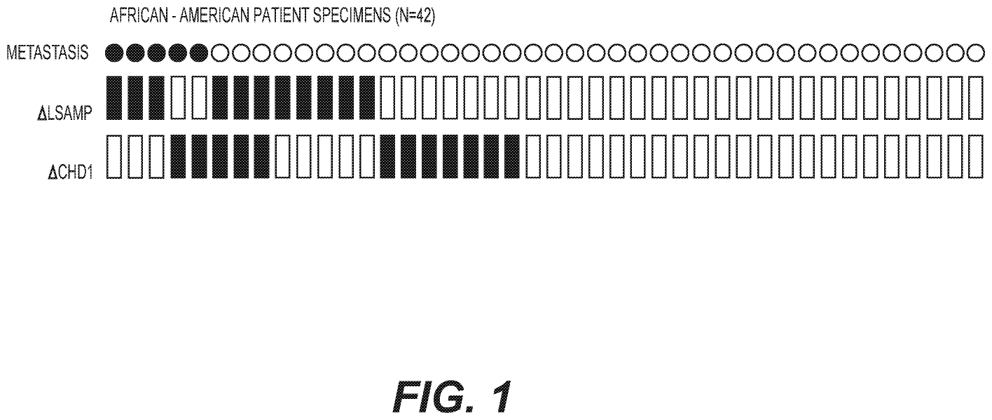

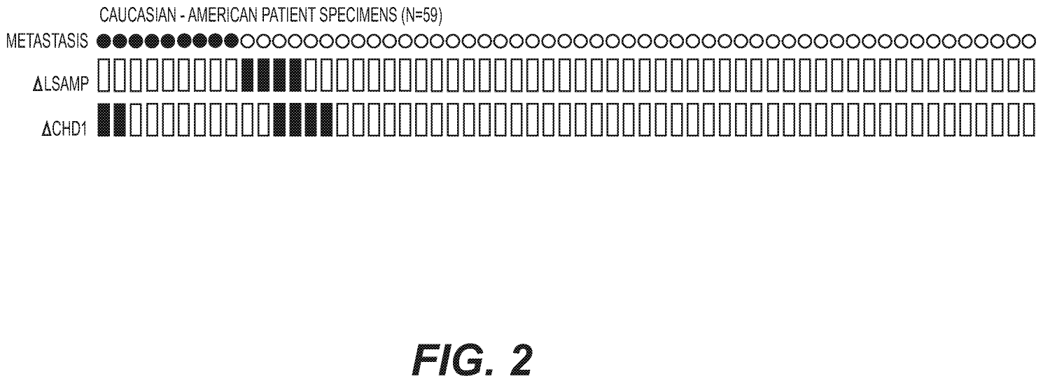

Whole genome sequence analysis of these prostate cancer samples identified genomic rearrangements resulting in the deletion of the PTEN gene, deletion of the LSAMP gene and/or deletion of the CHD1 gene. FIG. 1 schematically illustrates the results for the AD cohort, while FIG. 2 schematically illustrates the results for the CD cohort.

Of the 42 samples from subjects of AD, 23 of the subjects (55%) exhibited a future biochemical recurrence event, and of the 59 samples from subjects of CD, 33 of the subjects (56%) exhibited a future biochemical recurrence. As used herein, biochemical recurrence (BCR) is the measure of PSA rise that initiates hormonal ablations and/or chemotherapy treatment. A BCR event was defined as a post-radical prostatectomy serum PSA level greater than 0.2 ng/mL, measured no less than eight weeks after radical prostatectomy, followed by a successive, confirmatory PSA level greater than or equal to 0.2 ng/mL or the initiation of salvage radiation or hormonal therapy after a rising PSA level greater than or equal to 0.1 ng/mL. Patients who had an initial serum PSA greater than 0.2 ng/mL but no rise of PSA and no initiation of salvage therapy were classified into the non-BCR event category.

Out of the 23 samples from subjects of AD wherein the subject exhibited a future biochemical recurrence event, 9 (39%) were positive for the LSAMP deletion (i.e., the LSAMP gene was deleted), and 10 of the 23 samples (43%) were positive for the CHD1 deletion (i.e., the CHD1 gene was deleted). To the contrary, only 2 of the 33 samples (6%) from subjects of CD were positive for the LSAMP deletion, and 4 of the 33 samples (12%) were positive for the CHD1 deletion. Notably, 15 of the 23 samples (65%) from subjects of AD who exhibited a future biochemical recurrence were positive for either the LSAMP deletion or the CHD1 deletion. This is in contrast to 5 of the 33 samples (15%) from subjects of CD who exhibited a future biochemical recurrence and were positive for either the LSAMP deletion or the CHD1 deletion.

It was thus determined that a genomic rearrangement resulting in the deletion of either the LSAMP gene or the CHD1 gene was predictive of a future biochemical recurrent event for patients of AD, while such an LSAMP or CHD1 gene deletion was not predictive of a future biochemical recurrent event for patients of CD. However, due to mutual exclusivity between tumor foci, co-deletions of both CHD1 and LSAMP were rarely observed in the same patient. Only 3 (13%) of the 23 subjects from the AD cohort were positive for both CHD1 and LSAMP deletions, and 0 (0%) of the 33 subjects from the CD cohort were positive for both CHD1 and LSAMP deletions.

Fluorescence in situ hybridization analysis of these prostate cancer samples further identified genomic rearrangements resulting in the deletion of the PTEN gene. Out of the 23 samples from subjects of AD wherein the subject exhibited a future biochemical recurrence event, 11 (48%) were positive for the PTEN deletion (i.e., the PTEN gene was deleted) or positive for ERG oncoprotein by immunohistochemistry. In contrast, out of the 33 samples from subjects of CD wherein the subject exhibited a future biochemical recurrent event, 26 (79%) were positive for the PTEN deletion or positive for ERG oncoprotein. Out of the 23 samples from subjects of AD wherein the subject exhibited a future biochemical recurrent event, 2 (9%) were positive for both the PTEN deletion and positive for ERG expression, while out of the 33 samples from subjects of CD, 11 (33%) were positive for both the PTEN deletion and positive for ERG oncoprotein. It was thus determined that a genomic rearrangement resulting in the deletion of the PTEN gene was not predictive of a future biochemical recurrent event for patients of AD, while such a PTEN deletion was predictive of a future biochemical recurrent event for patients of CD. The results are summarized in Table 2 below.

TABLE 2
Biochemical Recurrence (BCR) in AD and CD cohorts

| | ΔCHD1 or | ΔPTEN or | ΔCHD1 and | ΔPTEN and |
|---|---|---|---|---|
| BCR (N = 56) | ΔLSAMP | ERG(+) | ΔLSAMP | ERG(+) |
| AD (N = 23) | 15 (65.2%) | 11 (47.8%) | 3 (13.0%) | 2 (8.7%) |
| CD (N = 33) | 5 (15.2%) | 26 (79%) | 0 (0.0%) | 11 (33.3%) |

Similarly, all of the subjects in the AD cohort who were determined to have a future bone metastasis (wherein the cancer has spread beyond the prostate gland and into bone tissue) were found to have at least one genetic alteration, i.e., either a CHD1 or an LSAMP gene deletion. Of the 5 bone metastasis subjects in the AD cohort, all 5 (100%) who exhibited a future bone metastasis were positive for either the LSAMP deletion or the CHD1 deletion. See FIG. 1. This is in contrast to 2 of the 9 samples (22%) from subjects in the CD cohort who exhibited a future bone metastasis and were positive for either the LSAMP deletion or the CHD1 deletion. See FIG. 2. As with future biochemical recurrence, co-deletions of both CHD1 and LSAMP were rare in future bone metastasis cases, constituting 0 (0%) of the samples from the both the AD and the CD cohorts. See FIGS. 1 and 2.

The prevalence of either deletion of PTEN or expression of ERG accounted for 2 (40%) of the 5 samples from the subjects in the AD cohort and 8 (89%) of the 9 samples from the subjects in the CD cohort. Additionally, it was found that the prevalence of both deletion of PTEN and expression of ERG accounted for 0 (0%) of the 5 samples from the subjects in the AD cohort and 6 (67%) of the 9 samples from the subjects in the CD cohort. The results are shown below in Table 3.

TABLE 3
Bone Metastatis (Met) in AD and CD cohorts

| | ΔCHD1 or | ΔPTEN or | ΔCHD1 and | ΔPTEN and |
|---|---|---|---|---|
| Met (N = 14) | ΔLSAMP | ERG(+) | ΔLSAMP | ERG(+) |
| AD (N = 5) | 5 (100%) | 2 (40%) | 0 (0.0%) | 0 (0.0%) |
| CD (N = 9) | 2 (22.2%) | 8 (88.9%) | 0 (0.0%) | 6 (66.7%) |

Finally, deletions of both CHD1 and LSAMP were detected in about 50% of the tumor foci having a higher Gleason score (Gleason score of 8 to 10), which was significantly higher than the prevalence of both deletions in groups having a Gleason score of 7 (30%) and a Gleason score of 6 (13%). To the contrary, the prevalence of genetic alterations association with the CD cohort (deletion of PTEN and expression of ERG) was found to be evenly distributed within the Gleason score groups. It was therefore concluded that deletions of CHD1 or LSAMP associated with the AD cohort tended to drive disease progression, biochemical recurrence, and bone metastasis, exceeding the predictive power of Gleason scores.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims. The claims are intended to cover the components and steps in any sequence which is effective to meet the objectives there intended, unless the context specifically indicates the contrary.

### REFERENCES

The following references are cited in the application and provide general information on the field of the invention and provide assays and other details discussed in the application. The following references are incorporated herein by reference in their entirety.
1. Siegel, R.; Miller, K.; Jemal, A. Cancer statistics, 2019. CA Cancer J Clin. 2019, 69, 7-34.
2. Chornokur, G.; Dalton, K.; Borysova, M. E.; Kumar, N. B. Disparities at presentation, diagnosis, treatment, and survival in African American men affected by prostate cancer. Prostate 2011, 71, 985-997.
3. Schwartz, K.; Powell, I. J.; Underwood, W., 3rd; George, J.; Yee, C.; Banerjee, M. Interplay of race, socioeconomic status, and treatment on survival of patients with prostate cancer. Urology 2009, 74, 1296-1302.
4. Major, J. M.; Oliver, M. N.; Doubeni, C. A.; Hollenbeck, A. R.; Graubard, B. I.; Sinha, R. Socioeconomic status, healthcare density, and risk of prostate cancer among African American and Caucasian men in a large prospective study. Cancer Causes Control 2012, 23, 1185-1191.
5. Sridhar, G.; Masho, S. W.; Adera, T.; Ramakrishnan, V.; Roberts, J. D. Do African American men have lower survival from prostate cancer compared with White men? A meta-analysis. Am. J Mens. Health 2010, 4, 189-206.
6. Cullen, J.; Brassell, S.; Chen, Y.; Porter, C.; L′Esperance, J.; Brand, T.; McLeod, D. G. Racial/ethnic patterns in prostate cancer outcomes in an active surveillance cohort. Prostate Cancer 2011, 2011, doi:10.1155/2011/234519.
7. Berger, A. D.; Satagopan, J.; Lee, P.; Taneja, S. S.; Osman, I. Differences in clinicopathologic features of prostate cancer between black and white patients treated in the 1990s and 2000s. Urology 2006, 67, 120-124.
8. Kheirandish, P.; Chinegwundoh, F. Ethnic differences in prostate cancer. Br. J. Cancer 2011, 105, 481-485.
9. Odedina, F. T.; Akinremi, T. O.; Chinegwundoh, F.; Roberts, R.; Yu, D.; Reams, R. R.; Freedman, M. L.; Rivers, B.; Green, B. L.; Kumar, N. Prostate cancer disparities in black men of African descent: A comparative literature review of prostate cancer burden among black men in the United States, Caribbean, United Kingdom, and West Africa. Infect. Agents Cancer 2009, 4, doi:10.1186/1750-9378-4S1-S2.
10. Heath, E. I.; Kaftan, M. W.; Powell, I. J.; Sakr, W.; Brand, T. C.; Rybicki, B. A.; Thompson, I. M.; Aronson, W. J.; Terris, M. K.; Kane, C. J.; et al. The effect of race/ethnicity on the accuracy of the 2001 Partin Tables for predicting pathologic stage of localized prostate cancer. Urology 2008, 71, 151-155.
11. Moul, J. W.; Sesterhenn, L A.; Connelly, R. R.; Douglas, T.; Srivastava, S.; Mostofi, F. K.; McLeod, D. G. Prostate-specific antigen values at the time of prostate cancer diagnosis in African-American men. JAMA 1995, 274, 1277-1281.
12. Tewari, A.; Horninger, W.; Badani, K. K.; Hasan, M.; Coon, S.; Crawford, E. D.; Gamito, E. J.; Wei, J.; Taub, D.; Montie, J.; et al. Racial differences in serum prostate-specific (PSA) doubling time, histopathological variables and long-term PSA recurrence between African-American and white American men undergoing radical prostatectomy for clinically localized prostate cancer. BJU Int. 2005, 96, 29-33.
13. Wallace, T. A.; Prueitt, R. L.; Yi, M.; Howe, T. M.; Gillespie, J. W.; Yfantis, H. G.; Stephens, R. M.; Caporaso, N. E.; Loffredo, C. A.; Ambs, S. Tumor immunobiological differences in prostate cancer between African-American and Caucasian-American men. Cancer Res. 2008, 68, 927-936.
14. Prensner, J. R.; Rubin, M. A.; Wei, J. T.; Chinnaiyan, A. M. Beyond PSA: The next generation of prostate cancer biomarkers. Sci. Transl. Med. 2012, 4, doi:10.1126/scitranslmed.3003180.
15. Rubin, M. A.; Maher, C. A.; Chinnaiyan, A. M. Common gene rearrangements in prostate cancer. J Clin. Oncol. 2011, 29, 3659-3668.
16. Sreenath, T. L.; Dobi, A.; Petrovics, G.; Srivastava, S. Oncogenic activation of ERG: A predominant mechanism in prostate cancer. J Carcinog. 2011, 11, 10-21.
17. Petrovics, G.; Liu, A.; Shaheduzzaman, S.; Furasato, B.; Sun, C.; Chen, Y.; Nau, M. Ravindranath, L.; Chen, Y.; Dobi, A.; et al. Frequent overexpression of ETS-related gene-1 (ERG1) in prostate cancer transcriptome. Oncogene 2005, 24, 3847-3852.
18. Tomlins, S. A.; Rhodes, D. R.; Perner, S.; Dhanasekaran, S. M.; Mehra, R.; Sun, X. W.; Varambally, S.; Cao, X.; Tchinda, J.; Kuefer, R.; et al. Recurrent fusion of TMPRSS2 and ETS transcription factor genes in prostate cancer. Science 2005, 310, 644-648.
19. Magi-Galluzzi, C.; Tsusuki, T.; Elson, P.; Simmerman, K.; LaFarque, C.; Esqueva, R.; Klein, E.; Rubin, M. A.; Zhou, M. TMPRSS2-ERG gene fusion prevalence and class are significantly different in prostate cancer of Caucasian, African-American and Japanese patients. Prostate 2011, 71, 489-497.
20. Rosen, P.; Pfister, D.; Young, D.; Petrovics, G.; Chen, Y.; Cullen, J.; Bohm, D.; Perner, S.; Dobi, A.; McLeod, D. G.; et al. Differences in frequency of ERG oncoprotein expression between index tumors of Caucasian and African American patients with prostate cancer. Urology 2012, 80, 749-753.
21. Hu, Y.; Dobi, A.; Sreenath, T.; Cook, C.; Tadase, A. Y.; Ravindranath, L.; Cullen, J.; Furusato, B.; Chen, Y.; Thanqapazham, R. L.; et al. Delineation of TMPRSS2-ERG splice variants in prostate cancer. Clin. Cancer Res. 2008, 14, 4719-4725.
22. Gary K Geiss, et al. (2008) Direct multiplexed measurement of gene expression with color-coded probe pairs, Nature Biotechnology 26:317-25.
23. Paolo Fortina and Saul Surrey, (2008) Digital mRNA Profiling, Nature Biotechnology 26:317-25.
Farrell J, Petrovics G, McLeod D G, Srivastava S.: Genetic and molecular differences in prostate carcinogenesis between African American and Caucasian American men. International Journal of Molecular Sciences. 2013; 14(8):15510-31.
25. Rodriquez-Suarez et al., Urine as a source for clinical proteome analysis: From discovery to clinical application, Biochimica et Biophysica Acta (2013).
26. Shi et al., Antibody-free, targeted mass-spectrometric approach for quantification of proteins at low picogram per milliliter levels in human plasma/serum, PNAS, 109(38):15395-15400 (2012).
27. Elentiboba-Johnson and Lim, Fusion peptides from oncogenic chimeric proteins as specific biomarkers of cancer, Mol Cell Proteomics, 12:2714 (2013).
28. Ras/Raf/MEK/ERK and PI3K/PTEN/Akt/mTOR Cascade Inhibitors: How Mutations Can Result in Therapy Resistance and How to Overcome Resistance, Oncotarget, 3(10):1068-1111 (2012).
29. Kuhn et al., High-resolution genomic profiling of adult and pediatric core-binding factor acute myeloid leukemia reveals new recurrent genomic alterations, Blood, 119(10):e67 (2012).
30. Pasic et al., Recurrent Focal Copy Number Changes and Loss of Heterozygosity Implicate Two Non-Coding RNAs and One Tumor Suppressor Gene at Chromosome 3q13.31 in Osteosarcoma, Cancer Research, 70(1):160-71 (2010).
31. Chen et al., The t(1;3) breakpoint-spanning genes LSAMP and NORE1 are involved in clear cell renal cell carcinomas, Cancer Cell, 4:405-413 (2003).
32. Ntougkos et al., Clin Cancer Res, 11:5764-5768 (2005).
33. Huang et al., Eur J Cancer 49:3729-37 (2013).
34. Mao et al., Cancer Res, 70:5207-5212 (2010).
35. Blattner et al., Neoplasia 16(1):14-20 (2014).
36. Khani et al., Clin Cancer Res 20(18):4925-34 (2014).
37. Farrell, et al., Predominance of ERG-negative high-grade prostate cancers in African American men, Mol Clin Onco 2: 982-986 (2014).

1.-4. (canceled)

5. A method of testing for the presence of genomic rearrangements in an LSAMP gene and a CHD1 gene in a biological sample obtained from a subject, the method comprising:
(a) assaying the biological sample to determine if it contains a first genomic rearrangement that results in deletion of an LSAMP gene, and
(b) assaying the biological sample to determine if it contains a second genomic rearrangement that results in deletion of a CHD1 gene,
wherein the subject is of African descent, and
wherein the biological sample comprises human prostate cells or nucleic acids isolated therefrom.

6. The method of claim 5, wherein the biological sample is a tissue sample, a cell sample, a blood sample, a serum sample, or a urine sample.

7. The method of claim 5, further comprising assaying the biological sample to determine if the biological sample contains a third genomic rearrangement that results in deletion of a PTEN gene in the biological sample.

8. The method of claim 5, further comprising assaying the biological sample to determine if the biological sample contains a TMPRSS2:ERG gene fusion in the biological sample.

9. The method of claim 5, wherein the first genomic rearrangement results from a genomic rearrangement on chromosome region 3q13 between a ZBTB20 gene and the LSAMP gene.

10. The method of claim 5, wherein the first genomic rearrangement comprises a deletion that spans the ZBTB20 and LSAMP genes.

11. The method of claim 5, further comprising measuring the expression of one or more of the following genes: PTEN, COL10A1, HOXC4, ESPL1, MMP9, ABCA13, PCDHGA1, AGSK1, ERG, AMACR, PCA3, or KLK3.

12. The method of claim 5, further comprising a step of performing confirmatory histological examination of prostate tissue from the subject, increasing the frequency of monitoring the subject for the development of prostate cancer or a more aggressive form of prostate cancer, or selecting a treatment regimen for the subject based on the detection of the presence of the first or the second genomic rearrangement.

13. The method of claim 5, further comprising a step of treating the subject with a treatment regimen if the presence of the first or second genomic rearrangement is detected in the biological sample obtained from the subject.

14. The method of claim 13, wherein the treatment regimen comprises at least one of surgery, radiation therapy, hormone therapy, chemotherapy, biological therapy, or high intensity focused ultrasound.

15. The method of claim 13, wherein the treatment regimen comprises at least one of radiation, poly(ADP-ribose) polymerase inhibitors and platinum-based agents.

16. The method of claim 12, further comprising a step of testing the biological sample from the subject to confirm that the biological sample does not contain a genomic rearrangement that results in deletion of a PTEN gene.

17. A kit for use in diagnosing or prognosing prostate cancer in a subject of African descent, the kit comprising at least two oligonucleotide probes, wherein the at least two oligonucleotide probes comprise a first oligonucleotide probe for detecting a first genomic rearrangement that results in a deletion of a human LSAMP gene and a second oligonucleotide probe for detecting a second genomic rearrangement that results in a deletion of a human CHD1 gene, wherein the kit contains oligonucleotide probes for detecting no more than 500 different genes.

18.-24. (canceled)

25. A method of treating a prostate cancer in a human patient, wherein the method comprises:
administering an effective amount of a treatment regimen to the human patient, wherein the treatment regimen comprises at least one of surgery, radiation therapy, hormone therapy, chemotherapy, biological therapy, or high intensity focused ultrasound, wherein the human patient is of African descent and prior to the administering step has been identified as having prostate cells that comprise at least one of a first genomic rearrangement that results in a deletion of an LSAMP gene and a second genomic rearrangement that results in a deletion of a CHD1 gene.

26. The method of claim 25, wherein the human patient is of African descent.

27. The method of claim 25, further comprising identifying the human patient as having prostate cells that do not contain a genomic rearrangement that results in deletion of a PTEN gene.

28. The method of claim 25, wherein the appropriate prostate cancer treatment or treatment regimen comprises administration of at least one of poly(ADP-ribose) polymerase inhibitors and platinum-based agents.


SEQUENCE LISTING (25 sequences)

SEQ ID NO: 1 - protein, 338 aa, Homo sapiens
MVRRVQPDRKQLPLVLLRLLCLLPTGLPVRSVDFNRGTDNITVRQGDTAILRCVVEDKNSKVAWLNRSGIIFAGHDKWSLDPRVELEKRHSLEYSLRIQKVDVYDEGSYTCSVQTQHEPKTSQVYLIVQVPPKISNISSDVTVNEGSNVTLVCMANGRPEPVITWRHLTPTGREFEGEEEYLEILGITREQSGKYECKAANEVSSADVKQVKVTVNYPPTITESKSNEATTGRQASLKCEASAVPAPDFEWYRDDTRINSANGLEIKSTEGQSSLTVTNVTEEHYGNYTCVAANKLGVTNASLVLFRPGSVRGINGSISLAVPLWLLAASLLCLLSKC

SEQ ID NO: 2 - DNA, 9478 bp, Homo sapiens
ggaggagggggagagaggctctgggttgctgctgcttctgctgctgctgctgctgtgtggctgtttctgtacactcactggcaggcttggtgccggctccctcgcccgcccgcccgccagcctgggaaagtgggttacagagcgaaggagctcagctcagacactggcagaggagcatccagtcacagagagaccaaacaagaaccctttcctttggcttcctcttcagctcttccagagggcttgctatttgcactctctcttttgaaattgtgttgcttttacttttcacccttctgcttgggttttatgagggctttgttaagtcttagagggaaaagagactgagcgagggaaagagagaggcaaagtggaaaggaccataaactggcaaagcccgctctgcgctcgctgtggatgaaagccccgtgttggtgaagcctctcctcgcgagcagcgcgcacccctccagagcaccccgcggacccgcacctcggcgtggccaccatggtcaggagagttcagccggatcggaaacagttgccactggtcctactgagattgctctgccttcttcccacaggactgcctgttcgcagcgtggattttaaccgaggcacggacaacatcaccgtgaggcagggggacacagccatcctcaggtgcgttgtagaagacaagaactcaaaggtggcctggttgaaccgttctggcatcatttttgctggacatgacaagtggtctctggacccacgggttgagctggagaaacgccattctctggaatacagcctccgaatccagaaggtggatgtctatgatgagggttcctacacttgctcagttcagacacagcatgagcccaagacctcccaagtttacttgatcgtacaagtcccaccaaagatctccaatatctcctcggatgtcactgtgaatgagggcagcaacgtgactctggtctgcatggccaatggccgtcctgaacctgttatcacctggagacaccttacaccaactggaagggaatttgaaggagaagaagaatatctggagatccttggcatcaccagggagcagtcaggcaaatatgagtgcaaagctgccaacgaggtctcctcggcggatgtcaaacaagtcaaggtcactgtgaactatcctcccactatcacagaatccaagagcaatgaagccaccacaggacgacaagcttcactcaaatgtgaggcctcggcagtgcctgcacctgactttgagtggtaccgggatgacactaggataaatagtgccaatggccttgagattaagagcacggagggccagtcttccctgacggtgaccaacgtcactgaggagcactacggcaactacacctgtgtggctgccaacaagctgggggtcaccaatgccagcctagtccttttcagacctgggtcggtgagaggaataaatggatccatcagtctggccgtaccactgtggctgctggcagcatctctgctctgccttctcagcaaatgttaatagaataaaaatttaaaaataatttaaaaaacacacaaaaatgcgtcacacagaatacagagagagagagacagagagagagagagagagagagatgggggagaccgtttatttcacaactttgtgtgtttatacatgaagggggaaataagaaagtgaagaagaaaatacaacatttaaaacaattttacagtccatcattaaaaatttatgtatcattcaggatggagaaggttctactgggatatgtttatatctactaagcaaatgtatgctgtgtaaagactacaccacactaaggacatctggatgctgtaaaaataagagaagaaccagatggatattaagccccccaacacacactttatccttccttccttcatcttttttcatctgtggggaagaaaataaggtcttgcctttggtgtttatatttccataaccttttaattctatttttcatttgagctgacttgtagccacttcagactatcaatggaatcttatgttgagcctttctctggctttccttcctccactatctctccaactttagagatcatcccctctccctccagtgcgttctatctcccccacacccaccctagatactcccttttcacccacctttcctccctcacctctcctcacctccaccccctccccagagcactagtcatgccgcaaatgctaggaagtgccattttcattttctccactgtgcgtgtgtgctcaagtctttcgctctcacgtgggtgtacatgtgtgtgagcgtgtgtgtgtctctctctaaagcatgccaagggaatggtccatgtgtacatagactcattgtgctgtagatactgtcctgcattgtaattgtgagatgcggctgtaacaagttgctgggggagatggcggggaaagaggcaaggagcagagtcctccctacatccatggctgtcacatggcatcagtgtgtattcaaaccaagctatgctccttccaagggcaggaccccatattcctcctagtcccatcatcagaaccgagtggggagtcactcagaatatcactgtaaatgaaagtgcctactatcgatggggtaagcaaacagcataaggaattatgacgtggacgaggtgacctaggagagaaaatttcagattttactctcatttcatgagtctgagggattcttatatttcctggcatttaacagggtaggccctgctccactgtgaaaatgagcagcatgtgttgagtaaatccccagaaacaggaaggtctccaagtgtcaactcccagtgaaagaatgatgaaccacttggagatcctaagcagccctgttttacctcctccctaatcttaaataacatttgtcccatgaattcccctgagcagagattgtttcctatttcagataaaatacagtgaaagtgagcaaggcagaaaaagtcaacagatgcccaggctcctactgtattctggagatactgtcagagctctaatacagagcactggccataatgaaaagcagttcactccttgtgctcctctgcagatgtttttcccagtgttctaggttaatgttttatttggttgcctgcataatccctgttctgtttcactgatggtgtttgcagcaccactgttcatggtggtccactgttatcctatgccagggtgctaagaattgcatgatattcatcttccctgctctatttaaatttacatctataagagtcatcttgacattaacactgaaatgtgatctaggtccttaaccaaaattgctgggcaacttgtaataaatttagacagaaattttatgagtaccacaaagcttggtgttaccacatcaccagaaggatttcttaggaaatgtcttgccgagagagctggctctctgcatatagatgtctttgtcagaaaaccaacccttgctctcacttacacagtagtaagcactgaaagtggttcagttcatgagaggacagagaattattttgagattatatttgaatgtaatcttgcagagccaaatatggtatgtcattaagttggaaccttgtaaatagctgttccatgttataaaatgagaaactttgtaactggaaaaaaagaaaggaaagaaggaaggaaggaaggaaggaaggcagggagggggggacggggaagggggggagggagggagggagggagggagggagggagggagggaggaaggaaggaaggaaggaaggaaggagaaaggaaaggaaggcaggagggagaaagatctaagtagcattgttaatttcttcaatttcttctaagggattttattgtttgttttagaagcttatcacagccttctttcatgattttgcagtttagacttgatacaaggaaaaattcagcttggggatggttaagagtgtttatagcagattctgacataggagagaaaacaaattctcatccaagaaggtagctagtaaaatatagggaaggtgagccatattcctatgcagcatcaatttattgacaatcaggtatttctcttaacagtttggtcttcttagttcaagaataaagggtatcatctttaataataagcattccccaaaaattgaagaggcagtcacacacttaagtgtgtggctttagaaaagcgcatgctaatttaaagatatacaggaagagaaaagtaggagttaagttggatgttgttagaagttggatgttagtattaccttcaggaacagatccccatggcatgtcacaggccttaattatatacctggctttcttattgtctccactttatcatgaggacaaggtcttggtttcatgggaggaacttctccattgaaataaatgtctgccatgtcagcaccgtttgttccctcagttttaatataatggaccatatattaaacataattaaacatatatttaaatgtggtgtttgcctgtgtctctagcaggatcttgaaattttaaaaatttgcttctggttcctgtttcagagaaaacattgtccccagaaatttcataggattgaaagtgttccctaagcagtgtgaacaatggaggaaaatatagtttagagaaaagtcagggaaaggtagggccagaggactgacaccaagaaatcattgaatctcaacataagacttcttggaatttagttaatcatattggaataaattccttcaagaatcttgtcccttggtaatcaaagtttgaaaccccgcactgaaaagcaccaactggttggaaataatatactgagaggagtgaaattcatcaattaatctgagtggctaatatatttaatatcctttgtatacaaagtaaaactccaccattcgtaaaaggaaatccttagacccaactttcagttaacaaaaacagaaatgactttgacccagggtgcttcctgaagaatgagaactatccagggctttacaactgcagaattgtaattatgctctgtgcaattgttgagcaaaggttttgccttgctggataaaaagtcttgtttgtttcgagacatgaaatccccatgtcttaaaagaactaaggcttatagaaaagcagatgggttttctctcaggaaggactgccccattgacctttgccttctcttccaagtcagacagacttctcgcttgccatgggcatttttttactacatagtcagactactggggctacttatagagaccttgtaaaagtactcgtgattttcacgttcttggaggaccaaacaaaaatctgtttctcctccaaaaatggacttacctcctttgcacacaaaagctaaactcctcagcatgaaattgtttgagttattactttaccaagttgtgagcttcttgaatcctccagagtcgcagattccatccctgagttggttgtggtttcactgtttctattggctattctccctgaatttttcattttgttctttgcagggctcgaattatttgtggaaaacaataatatatatgtgtgtgtattttttatctttatagatgctatatttacaataatgtatgtattataagacaaattaagaataatgttttgatcttaaaaggaagaaaagtactgaatttggttgtttagaaagaaaatctatgctcacgtagaaagacatagagccccactttttccgttttgtaattatttggcgaaaagaaatttgcttatagactatgtttaatgggattaaccatgtcctcatttttcttttcatcctcatacacttttagcctgcatttagtcttggttaccaatatcattttttaagagaaatgtaagtacagtgctatatcttacctacataaactattaatattttgaagacaaatgtgaaacacaccacaaaaatggtgagataagaaacaaaaatgcagtttaggaagcctcctccttgcttaaatgtttagaatatttcttctccaaagactgcatttgcctcagtgatgtaaattttccatcatggttggcataatatctgtaaacatctcactaaatgcaaaatggagtttacatttatgtgcatactagcagaaaagaagtaaactattctcatttgcatgtagctatgctgttcaaatgtcgccaaccaaaatttaggaaagaatttgttttcacccagcatgtacatctcacttttctcttggcaaggaagctggtgttaacgttgggtttaggtttaacatttacaccagcgaaaatgtttatgaatattatggaaaacttattttaaaccttgatttcttttgagcacatttacatgctgcgtgctgattaataaattaggcaccaatcatgtgtaaatcaatgtaaatcactagtttatgtacataatataaactatgtaaacttcatttttcatgcagtgcccaactactgctaaggtttactatgattcttaaggaaaaaaaaatcaaataaaaataaataaaaactgaaacgtttcacagttcagaatcggaagaattacgactaaagctccaaatatgaggttgccttagccaaaggaagcagacccacaggaacagttcaaggtttatatcctgctcaagtcacctctttggtctttcaggatctaagtggagattgtcccaactgttgctgtagttgtctcacccgaccccaaagcaagggaaatagagggaaaggttttaagggctatatgtctgctgtatccactcccagctatcttctttttactcctttctcaccatctaagatgtcttatttaaatagctccaaggaagtgacctaaaccttgatgagcaaaatattactcagtttttattttccattcaacaaaagcagtgggaaagcttgccatctggattctaagaaattgtgcaatataaaaaatgttatatcctcgagaaatatcttgctgagtcaccctaggaaataactaccttttatttatctggtagcctaatgtttccacacatttatcctgaatattgcaagtgagggactgaatcatttttaattgggagttatctttctcaggcatgttctcttggagtctttttgaagtgcctgacacgtgtaacaggatgacatatatatcattcctacaatatgaacaactgttacataaaaaattatcaaggagatgatttcaggaaacaagtgaactttctgcaaagacttataaaaattttaggtcaaattaacttcaggctttaaatgcactaatctacctaagagaaaaaaaaagaaaaaaaacaggaaggagttttaagggctcatgtgcctgttgtatcaacccccaagtgtcttcttggtactgcttcctcatcatctaagataaactgacaactttaaagtgaggtagaaggtgtattgaattgggagtcaggagaattgggttctaaccccaagttcagccacaaataaagctgtgagacattggcaagtcatttaactttcctgagtcttggttttctcatcctgaaagtgagggattcggctgacgtctctaaaatctctttcaactctaacctttattctgaataagaatttaatattcacttagtgctgtgcccagcactgtttgtaaacagcagctgtttgttatctctagtttggtctctgtattctcatcacttctcagaactatcattctaccgtcttcatttctaaacccaaaactgctagaatacagggactctggactgggtctgtaaattttttctgatcaaaactttatagcagtgtagagaagggacacattcaaattacactaaggacattgacatagctggggttgtttccttgtttatattataaaacctaaatgtggaactatattctaataatctttcataggaaggaaaatagccagactgggtattatgcatgtaacaaatgaggacattgtgcataagaaaggaaacattagttttctgtcatcctgggccaagtacctcattacagtaaatgtgtgtctttggaaactctttgcttgtgctgatggcggtaagcatggggtcccaggcaggttcaaaggctgaactgtaagaaatgggcaagacaatacattttgttttggaaggaatttctcatgggataagtttcccaaagcttgaattataggctatgaaataaagcaaatagatggagagaaaacaagtattgttttcaaaaagtacaagtcaattctatttaaagaagacaagctgaaaataaaacaaaaataaacacaatttaggaggttacagagttgaagacagtatgaattgttgtgaaggccaaaatcaaatgtgaaagttaggttctctgagaaaagggtaagcagaaaggatgatttctcaagcaattaataaggaattattttcttgtgccatgttctagatgcattgagcacagatcctcttgtcctaagctgtcctagaggctcaggttagcatctatccaaagttgtcctttgattttattgtctgaaagaacagaaggcatcagagtttccagtcactgaagagtagggtttgttcatcacttcccagcaatcacatcactttgtgtaggtaaggatatatgatgtgcttagattacttatgaagctctctctaagtgggagaatgacctgtccatgggacaactccccgttttcatggtcatttcagaagtacctctttttgggcagtgctcctggatctacttctacagccacattctactctgcacaatcctccctatgtaaagccaggcacagtacaaatatgcttcttgcaagtgaagaaaacccatggaagtcctagcttcatggcacgctgcagcaatcccaagctaccaggagcctcttttgaacccacttccctaagtctttgctcttcaccagagaatggaaattgttcatcctggtgaactgtggccaagttctgctccctaagtatttacttggagtagggaggttaaagggaagaaattcagggggagagaagcaaaagagaacacttccaactccctcccccatctcccaatgctccccaccttccttatcactgctctactgaagggtgtataaatcctgctcttggttagaattctccttattaacagtgttatatacatataaatatatatataaatatattccttttttcagccctgtagacatgaactgatcttcccttgaagatacaaacacatggccattttttgtttgggattttttgtttttcaaggtttttcatttttgtttattaggtggatttttttccctgggtactagctctgtgaaggagataaaaagcgcaatgtgtgttaaaaaaaaaaaattaaaattaaaatgaaaaaaagctttttttcttttctttttaaatgtatttaaattctgtttctctcttctgttactttacacgtatgaatgctctgctcttctgtgatcttaaaacaaaatgaaataaacgtgaaaaggagatgtgtcttcattgacctgtca

SEQ ID NO: 3 - DNA, 7895 bp, Homo sapiens
aagcggcatcacaggctgtcaacaaccgggtctctattttctctcctcctccggcgccccgcccgtgcgggacatcagctcccaggatgcgtcaatcgcagatcgcccaagcgtaagaggtggcgagaggccaccacggttgcccaacagttgcgttcccaggctgcctggtgtgacgcagcagcccccgcccggaccctgaggcctggcgctccgactgagccgaggcctgtgctacccgacactccttgcgacccggccccttaggcttattcagcgcggcccttcccggcttcccgagtcccatcgcagtctctccccacccccgttcctccccggttccttgtcagtgtccgccgcgatgtccttgcgcaaccatcccaagtgacattagcgcccatacccgcacggagcgagcctgaagctcccacttcccggaattcaacgccgggccgctaagggcgggcacccaagcgaggatcagcggactaaggccgcgaccagagtccagcggtctccgcgcagtcagagtccgacagcctctgcgcatccgccgtgcgcactcccattcgtgcgcgtgcgcgcactcgccccccccgccccgccccagcaccaatcgcttttttcctacgcccacgacccttcaacgtagtccctcccgcctcccgcgcgcgagcagaaaggcttcggcgtgcgcgcgcccgcccgcctcgcgctctgtcgggaaggggagtgagcagaggcgcttacgtcgcgcgggaggcggaggcgacggcgacctcggtggcggcggcggctacgacggagacggttgcgctcgcttgctttctcggcgtcatggcggctgccgggggcagataagcggagggagcgggagcgcgcgcgcgacggcggcggcggcggcggcgacgactggttacttatagctcttgctgccctcgcccttggtgcttcaataatgaattgttagctccactccgcaggggatcgcgcgttggtgctgcggaccggggcttccccttcccctccgcgtctgtccccttccccctcccccgcacggactcttgcttaccccgagacgccggagccctaggactgggcggcggagatcccgggcctgggcgcgggggaggccgtccacccgagtcggctccctccgccccgccaagcccgccggtgaatcaaaggagagtcccagaaaacctgtgactgttgaagaaaattcatctgtgaatttttatattcaaggagtcagtatttatattcatcttttaaactgggaagatttatattttactttaaaacttcttgataataatttacaatgaatggacacagtgatgaagaaagtgttagaaacagtagtggagaatcaagccagtcggatgatgattctgggtcagcttcaggctctggatctggttcgagttctggaagcagtagtgatggaagcagtagccagtcaggtagcagtgactctgactccggatctgaatcaggcagtcagtcagagtctgagtcagacacttcccgagaaaacaaagttcaagcaaaaccaccgaaagttgatggagctgagttttggaaatctagtcctagtattctggccgttcagagatctgcaatcctcaagaagcagcaacagcagcagcagcaacaacaacatcaagcctcatctaatagcggatcagaagaggattcctctagcagtgaagattccgatgactcatcaagtgaggtcaaaaggaaaaagcataaagatgaagattggcaaatgtctgggtcaggatctccatctcagtctggttcagattcagaatctgaagaagagagagagaaaagcagttgtgatgaaacagaatctgattatgagccaaaaaacaaagtcaaaagcagaaaacctcaaaatagatctaagtcaaaaaatggaaagaagattcttggacaaaaaaagagacagattgattcatctgaggaggatgatgatgaagaagattatgataatgataaaagaagttctcgtcgccaagcaactgttaatgttagctataaggaggatgaagaaatgaaaacagattctgatgacctactggaagtctgtggagaggatgttcctcaacctgaggaagaggaatttgaaaccatagaaagatttatggattgtcggattgggagaaaaggagctactggtgctactacaaccatctatgcagttgaagcagatggtgacccaaatgcaggctttgaaaaaaacaaagaaccaggagagattcagtatttaattaaatggaaaggatggtcccatatccacaacacttgggagacagaagaaaccctcaagcagcagaatgttagaggaatgaaaaaattggataattataagaaaaaagatcaggaaacaaaaagatggttgaaaaatgcctctccagaagatgtggaatattataattgccagcaagaacttacagatgatctacataaacagtatcaaatagtggaacgtataattgctcattccaatcaaaagtcagcagctggttatcctgattattactgcaaatggcagggccttccatactcagagtgcagctgggaagatggagctctcatttccaaaaagtttcaagcatgcattgatgagtattttagcaggaaccaatcaaaaaccactccttttaaagattgcaaagtattaaaacaaaggccaaggtttgtagccctgaagaagcagccatcctatattggaggacatgagggcttagaattaagagattatcaactgaatggtttaaattggcttgctcattcttggtgcaaaggaaatagttgcatactcgctgatgaaatgggccttggaaaaacaatacagacgatctcatttctgaattatttgtttcatgaacatcaattatatggaccttttttattggtagtaccgctctccactcttacttcctggcaaagggaaattcagacttgggcttctcaaatgaatgctgtggtttatttaggtgacattaacagcagaaacatgataagaactcatgaatggacgcatcatcagaccaaacggttaaaatttaatatattgttaacaacttatgaaattttattaaaagataaggcattccttggaggtctaaattgggcatttataggtgttgatgaagcacaccgattaaagaatgatgactcccttctgtataaaactttaatagattttaaatccaatcatcgtctccttatcactggaactcctctacagaattccctcaaagagctctggtctttgctacatttcattatgccagaaaagttttcttcctgggaagattttgaagaagaacatggcaaagggagagaatatggttatgcaagccttcacaaggagcttgagccatttctgttacgccgagttaagaaagatgtggaaaaatctcttcctgccaaggttgagcagattttaagaatggaaatgagtgctttacagaaacaatattacaaatggattttaactaggaattacaaagccctcagcaaaggttccaagggcagtacctcaggctttttgaacattatgatggagctaaagaaatgttgtaaccattgctacctcattaaaccaccagataataatgaattctataataaacaggaggccttacaacacttaattcgtagtagcggaaaattgattcttcttgacaagctattaattcgcctaagagaacgaggcaatcgagttcttattttttcacaaatggtgcggatgttagatatacttgcagaatatttgaaatatcgtcaattcccctttcaaagattagatggatcaataaaaggagaactgaggaaacaagctctagatcattttaatgctgagggatcagaggatttttgctttttgctgtccacaagagctggaggtctagggattaatttagcctctgctgacactgttgttatatttgattccgattggaatccacagaatgatcttcaggcacaggctagagcccatcgaattgggcaaaagaaacaggtgaatatttatcgtctagttacaaagggatcagttgaagaagatattcttgaaagggcgaaaaagaagatggttttagatcatcttgtaattcaaagaatggacacaactgggaagacagtactacatacaggttctgccccatcaagttctactcctttcaataaagaagagttatcagccattttaaagtttggtgctgaagaactttttaaggaacctgaaggagaagaacaagagccccaggaaatggatatagatgaaatcttgaagagagctgaaactcatgaaaatgaaccaggtcctttaactgtaggagatgaattgctttcccagttcaaggttgccaacttctcaaatatggatgaggatgacattgagttggaacctgaaagaaattcaaagaattgggaggaaattattccagaagatcaaagaagacgattagaagaagaagaaagacaaaaggaacttgaagaaatttatatgctcccaagaatgagaaattgtgcaaaacagattagtttcaatggaagtgaagggaggcgcagtagaagtaggagatactctggatctgatagtgattccatctcagaagggaaaaggccaaagaaacgtggaagaccacggactattcctcgggagaatattaaaggatttagtgatgcagaaattaggcggtttatcaagagctataagaaatttggtggtcctctggaaagattagatgcaattgctcgagatgctgagttagttgataagtcagaaacagaccttagacgactgggagaattggtacataatggttgcattaaagcattaaaggatagttcttcaggaacagaacgaacaggtggtagactcggaaaagtgaagggtccaacattccgaatatcaggagtacaggtgaatgccaaactagtcatctcccatgaagaagaattaatacctttgcacaaatccattccttctgatccagaagaaagaaagcagtatactatcccatgccacacaaaggcagctcattttgatatagactggggcaaagaagatgattccaatttgttaattggcatctatgaatatggatatggaagctgggaaatgattaaaatggatcctgacctcagtctaacacacaagattcttccagatgatcccgataaaaaaccacaagcaaaacagttgcagacccgtgcagactacctcatcaaattacttagtagagatcttgcaaaaaaagaagctctttctggtgcgggaagttcaaagaggagaaaagcaagagctaagaagaataaagcaatgaagtctataaaagtgaaagaggaaataaagagtgattcttctcctctgccttcagagaagtctgatgaagatgatgataaagatgagatcagttctgtgaaacatccaaataaaaaaattaaaacagaaagagacagtgaagaaaaacctgagccagatgtttatataaagaaggaaccagaagaaaagagggaagcaaaagaaaaggagaataaaaaagaacttaaaagggagataaaagaaaaagaggataagaaagatataaaggaaaaagattttaaagaaaaaagagaaaacaaagtaaaagaagctatacagaaagaaaaagacataaaggaagaaaagttgagtgaatccaagtctgatggtagggaaagatccaagaaatcttcagtgtcagatgctccagttcatatcacggcaagtggtgaaccagttcccatttctgaagaatctgaagagctggatcagaagacattcagcatttgtaaagaaagaatgaggcctgttaaagcagctttgaaacaacttgataggcctgagaaaggcctttcagaaagagaacaactagagcatactagacaatgtttaataaaaattggagaccatatcacagaatgtctaaaagagtatacaaatcctgaacaaattaagcaatggagaaaaaacctgtggatttttgtatctaagtttactgaatttgatgcaagaaaattacataaattatataagcatgctattaaaaaacggcaggagtctcagcaaaacagtgatcaaaacagcaacttgaatcctcacgtgattagaaatccagatgtggaaagattaaaagagaatacaaatcacgatgatagcagcagggacagttattcctctgatagacacttaactcagtaccatgatcatcataaagaccgacatcagggagattcttacaaaaaaagtgattccaggaaaagaccctattcttcttttagtaatggtaaagaccatcgtgattgggatcactacaagcaagacagcagatattacagtgacagagagaaacacagaaaactggatgatcacaggagtagagatcacaggtcaaatttggaaggaagtttaaaagatagatctcattctgatcatcgttctcactcagatcatcggttacattcagaccaccggtcaagttctgaatatacgcaccataaatcttccagggattataggtatcactcagactggcaaatggaccacagagcttccagcagtggccctaggtcaccactagatcagagatctccttatggctccagatctccatttgaacattcagttgaacacaaaagtacaccggagcatacctggagtagtcggaaaacataacaaaaactgatacttcgtctttctggacttttcttttagccatatatcataaaccaacacagtaattgccttacatgacttgaaagatataaacagatcttctatcagtagcagtattgttacttctttccaggatgcaaggtctattatcccaacagaagagaaaatatttttatatttaaggattatgctgcactgtactacaaaattgtagtacttttttttgttttcttttttaaagaaatggaaaatgtttactattacagggacctcaacactgccctcccatacaggctggataaaactgtttttaagtcagtgattttagactgacctccatttaaattatgtttatatatgaactttactctgacctgtgatcatgtttcaggaaggaatgaaagagagttctttcttaataaagaaaaacactcaaggactttgttcatttccaaagctacttgtttacattgtacactgcgaccaccttgccgcttttcatcacaagcttgaatatttaaattctgtacttatatctgtaaaatagccaggaatttcctgtttgtgatctattatgcctttttacaaaaaaaatggctgtaaattattgtaaatattaaaggaactttccttacttccttccctttctcaggctttttttgactgttcctttccctaccaactcaggccttcttattaaaaaaaaaaaaatcagtgtaataacactttttaatgatttgtcttgatggaatcattgtttagaatgtaaaaatggggaaaggggccacttaattccattagtcctctttttatactgaatattttattagatacatgttattcccttttttttccttttttagtcaatattgtgtttgtagttttaaaaaatggcgagatatgtaaaatctaaactgcatgctctggaaacacttttttcagatgcatctggtttaaaagggtaggtgtataaacacttttcagaatccaaaacggccaaaagttattgtaaatccgtttgttttcccgttttatgtgggcaataatgtcaaatgtgctatgcagccaggttaacattttagataaacttgattgacttttaatataaactgttacaatgcacactgattgtatataaaaacgttatatatgacaaattaaatttaagaaaaag

SEQ ID NO: 4 - protein, 741 aa, Homo sapiens
MLERKKPKTAENQKASEENEITQPGGSSAKPGLPCLNFEAVLSPDPALIHSTHSLTNSHAHTGSSDCDISCKGMTERIHSINLHNFSNSVLETLNEQRNRGHFCDVTVRIHGSMLRAHRCVLAAGSPFFQDKLLLGYSDIEIPSVVSVQSVQKLIDFMYSGVLRVSQSEALQILTAASILQIKTVIDECTRIVSQNVGDVFPGIQDSGQDTPRGTPESGTSGQSSDTESGYLQSHPQHSVDRIYSALYACSMQNGSGERSFYSGAVVSHHETALGLPRDHHMEDPSWITRIHERSQQMERYLSTTPETTHCRKQPRPVRIQTLVGNIHIKQEMEDDYDYYGQQRVQILERNESEECTEDTDQAEGTESEPKGESFDSGVSSSIGTEPDSVEQQFGPGAARDSQAEPTQPEQAAEAPAEGGPQTNQLETGASSPERSNEVEMDSTVITVSNSSDKSVLQQPSVNTSIGQPLPSTQLYLRQTETLTSNLRMPLTLTSNTQVIGTAGNTYLPALFTTQPAGSGPKPFLFSLPQPLAGQQTQFVTVSQPGLSTFTAQLPAPQPLASSAGHSTASGQGEKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

SEQ ID NO: 5 - DNA, 3317 bp, Homo sapiens
gttgggaaacagcccagtggtataaggatgaggaaactgaagcccagagaggtgaagtgaggtgcccaaggccacacagcaagttagaggcacagctagtacggtagctcaagtctcctgactcccagtccagtgctcctcccattactccacgagtcctgtctctaagcttcctgacaaatgctagaacggaagaaacccaagacagctgaaaaccagaaggcatctgaggagaatgagattactcagccgggtggatccagcgccaagccgggccttccctgcctgaactttgaagctgttttgtctccagacccagccctcatccactcaacacattcactgacaaactctcacgctcacaccgggtcatctgattgtgacatcagttgcaaggggatgaccgagcgcattcacagcatcaaccttcacaacttcagcaattccgtgctcgagaccctcaacgagcagcgcaaccgtggccacttctgtgacgtaacggtgcgcatccacgggagcatgctgcgcgcacaccgctgcgtgctggcagccggcagccccttcttccaggacaaactgctgcttggctacagcgacatcgagatcccgtcggtggtgtcagtgcagtcagtgcaaaagctcattgacttcatgtacagcggcgtgctacgggtctcgcagtcggaagctctgcagatcctcacggccgccagcatcctgcagatcaaaacagtcatcgacgagtgcacgcgcatcgtgtcacagaacgtgggcgatgtgttcccggggatccaggactcgggccaggacacgccgcggggcactcccgagtcaggcacgtcaggccagagcagcgacacggagtcgggctacctgcagagccacccacagcacagcgtggacaggatctactcggcactctacgcgtgctccatgcagaatggcagcggcgagcgctctttttacagcggcgcagtggtcagccaccacgagactgcgctcggcctgccccgcgaccaccacatggaagaccccagctggatcacacgcatccatgagcgctcgcagcagatggagcgctacctgtccaccacccccgagaccacgcactgccgcaagcagccccggcctgtgcgcatccagaccctagtgggcaacatccacatcaagcaggagatggaggacgattacgactactacgggcagcaaagggtgcagatcctggaacgcaacgaatccgaggagtgcacggaagacacagaccaggccgagggcaccgagagtgagcccaaaggtgaaagcttcgactcgggcgtcagctcctccataggcaccgagcctgactcggtggagcagcagtttgggcctggggcggcgcgggacagccaggctgaacccacccaacccgagcaggctgcagaagcccccgctgagggtggtccgcagacaaaccagctagaaacaggtgcttcctctccggagagaagcaatgaagtggagatggacagcactgttatcactgtcagcaacagctccgacaagagcgtcctacaacagccttcggtcaacacgtccatcgggcagccattgccaagtacccagctctacttacgccagacagaaaccctcaccagcaacctgaggatgcctctgaccttgaccagcaacacgcaggtcattggcacagctggcaacacctacctgccagccctcttcactacccagcccgcgggcagtggccccaagcctttcctcttcagcctgccacagcccctggcaggccagcagacccagtttgtgacagtgtcccagcccggtctgtcgacctttactgcacagctgccagcgccacagcccctggcctcatccgcaggccacagcacagccagtgggcaaggcgaaaaaaagccttatgagtgcactctctgcaacaagactttcaccgccaaacagaactacgtcaagcacatgttcgtacacacaggtgagaagccccaccaatgcagcatctgttggcgctccttctccttaaaggattaccttatcaagcacatggtgacacacacaggagtgagggcataccagtgtagtatctgcaacaagcgcttcacccagaagagctccctcaacgtgcacatgcgcctccaccggggagagaagtcctacgagtgctacatctgcaaaaagaagttctctcacaagaccctcctggagcgacacgtggccctgcacagtgccagcaatgggaccccccctgcaggcacacccccaggtgcccgcgctggccccccaggcgtggtggcctgcacggaggggaccacttacgtctgctccgtctgcccagcaaagtttgaccaaatcgagcagttcaacgaccacatgaggatgcatgtgtctgacggataagtagtatctttctctctttcttatgaacaaaacaaaacaacaacaaaaaacaaacaaacaaaaaagctatggcactagaatttaagaaatgttttggtttcatttttactttctgtttttgtttttgtttcgtttcattttgtactacatgaagaactgttttttgcctgctggtacattacatttccggaggcttgggtgaataatagttttcccagtctccctcggatggtggccttaaggcctggtagtgcttcaagaggtccactggttggatctctagctactggcctctaaatacaacccttctttacaaaaaaatcttttaaaaaaaagtaaaaaaaaaaaaaaaatttccacttgtgaagagcactacaaaaaatatataacaaaatctaaaaggcctactgtctttaagtacaccgcttgcagtgtttcagtggacattttcacaattctggccgcttggacttcacagtaaccagttaaaactgtggaatatcacttctggttgaaaacccagaggaaaggccctgctgttttccacctaccacgttgtctgatttcataaaagggctgtgggggtgggaagggcagtgggttcggtggtgtgggaaagaaagacgaatggcaggcttcttccccagattctgcccgggtccacacaccctggcccaccttctccatatccccctcttgcagcagaagccaggaagacttggacaagcaacaagcaacagtggctatcgtatttattcagtgtcttcgctgagccacagcctcagcacaatcaagagggactttcatgaaaggcaggaatgcagataaaacaaagatatcagaaatttgcacctatgtttctaggtacaagagaaggattatttccaacaatctttgcaaaaaaaaaaaaa

SEQ ID NO: 6 - protein, 668 aa, Homo sapiens
MTERIHSINLHNFSNSVLETLNEQRNRGHFCDVTVRIHGSMLRAHRCVLAAGSPFFQDKLLLGYSDIEIPSVVSVQSVQKLIDFMYSGVLRVSQSEALQILTAASILQIKTVIDECTRIVSQNVGDVFPGIQDSGQDTPRGTPESGTSGQSSDTESGYLQSHPQHSVDRIYSALYACSMQNGSGERSFYSGAVVSHHETALGLPRDHHMEDPSWITRIHERSQQMERYLSTTPETTHCRKQPRPVRIQTLVGNIHIKQEMEDDYDYYGQQRVQILERNESEECTEDTDQAEGTESEPKGESFDSGVSSSIGTEPDSVEQQFGPGAARDSQAEPTQPEQAAEAPAEGGPQTNQLETGASSPERSNEVEMDSTVITVSNSSDKSVLQQPSVNTSIGQPLPSTQLYLRQTETLTSNLRMPLTLTSNTQVIGTAGNTYLPALFTTQPAGSGPKPFLFSLPQPLAGQQTQFVTVSQPGLSTFTAQLPAPQPLASSAGHSTASGQGEKKPYECTLCNKTFTAKQNYVKHMFVHTGEKPHQCSICWRSFSLKDYLIKHMVTHTGVRAYQCSICNKRFTQKSSLNVHMRLHRGEKSYECYICKKKFSHKTLLERHVALHSASNGTPPAGTPPGARAGPPGVVACTEGTTYVCSVCPAKFDQIEQFNDHMRMHVSDG

SEQ ID NO: 7 - DNA, 3541 bp, Homo sapiens
gaagaagtaggggcggggggaagtttaggagttgaggaaagaagattaaagagcgcgaggagacaaataaaaagaagtgttaagaattgcctttgggactctgaaggctgaagaattgatgaattgcaagtttgtgccccatagctgcacagactgcctgaagttacatttagagactgaaatcactgcaccttaaaaacaaaagattgagctgcactgtattcctaatgtttcatcattactaacaggatattcctcatgacattgctgtctgatctttgaccatcagtctgtgacctgccccttctctttacatgcagccgctctctgctccctgccccaatgaacatctgcactaggcccaagccttggagtaatttacctgaagagtgacaccattgattttgaaactactgaagaaacccaagacagctgaaaaccagaaggcatctgaggagaatgagattactcagccgggtggatccagcgccaagccgggccttccctgcctgaactttgaagctgttttgtctccagacccagccctcatccactcaacacattcactgacaaactctcacgctcacaccgggtcatctgattgtgacatcagttgcaaggggatgaccgagcgcattcacagcatcaaccttcacaacttcagcaattccgtgctcgagaccctcaacgagcagcgcaaccgtggccacttctgtgacgtaacggtgcgcatccacgggagcatgctgcgcgcacaccgctgcgtgctggcagccggcagccccttcttccaggacaaactgctgcttggctacagcgacatcgagatcccgtcggtggtgtcagtgcagtcagtgcaaaagctcattgacttcatgtacagcggcgtgctacgggtctcgcagtcggaagctctgcagatcctcacggccgccagcatcctgcagatcaaaacagtcatcgacgagtgcacgcgcatcgtgtcacagaacgtgggcgatgtgttcccggggatccaggactcgggccaggacacgccgcggggcactcccgagtcaggcacgtcaggccagagcagcgacacggagtcgggctacctgcagagccacccacagcacagcgtggacaggatctactcggcactctacgcgtgctccatgcagaatggcagcggcgagcgctctttttacagcggcgcagtggtcagccaccacgagactgcgctcggcctgccccgcgaccaccacatggaagaccccagctggatcacacgcatccatgagcgctcgcagcagatggagcgctacctgtccaccacccccgagaccacgcactgccgcaagcagccccggcctgtgcgcatccagaccctagtgggcaacatccacatcaagcaggagatggaggacgattacgactactacgggcagcaaagggtgcagatcctggaacgcaacgaatccgaggagtgcacggaagacacagaccaggccgagggcaccgagagtgagcccaaaggtgaaagcttcgactcgggcgtcagctcctccataggcaccgagcctgactcggtggagcagcagtttgggcctggggcggcgcgggacagccaggctgaacccacccaacccgagcaggctgcagaagcccccgctgagggtggtccgcagacaaaccagctagaaacaggtgcttcctctccggagagaagcaatgaagtggagatggacagcactgttatcactgtcagcaacagctccgacaagagcgtcctacaacagccttcggtcaacacgtccatcgggcagccattgccaagtacccagctctacttacgccagacagaaaccctcaccagcaacctgaggatgcctctgaccttgaccagcaacacgcaggtcattggcacagctggcaacacctacctgccagccctcttcactacccagcccgcgggcagtggccccaagcctttcctcttcagcctgccacagcccctggcaggccagcagacccagtttgtgacagtgtcccagcccggtctgtcgacctttactgcacagctgccagcgccacagcccctggcctcatccgcaggccacagcacagccagtgggcaaggcgaaaaaaagccttatgagtgcactctctgcaacaagactttcaccgccaaacagaactacgtcaagcacatgttcgtacacacaggtgagaagccccaccaatgcagcatctgttggcgctccttctccttaaaggattaccttatcaagcacatggtgacacacacaggagtgagggcataccagtgtagtatctgcaacaagcgcttcacccagaagagctccctcaacgtgcacatgcgcctccaccggggagagaagtcctacgagtgctacatctgcaaaaagaagttctctcacaagaccctcctggagcgacacgtggccctgcacagtgccagcaatgggaccccccctgcaggcacacccccaggtgcccgcgctggccccccaggcgtggtggcctgcacggaggggaccacttacgtctgctccgtctgcccagcaaagtttgaccaaatcgagcagttcaacgaccacatgaggatgcatgtgtctgacggataagtagtatctttctctctttcttatgaacaaaacaaaacaacaacaaaaaacaaacaaacaaaaaagctatggcactagaatttaagaaatgttttggtttcatttttactttctgtttttgtttttgtttcgtttcattttgtactacatgaagaactgttttttgcctgctggtacattacatttccggaggcttgggtgaataatagttttcccagtctccctcggatggtggccttaaggcctggtagtgcttcaagaggtccactggttggatctctagctactggcctctaaatacaacccttctttacaaaaaaatcttttaaaaaaaagtaaaaaaaaaaaaaaaatttccacttgtgaagagcactacaaaaaatatataacaaaatctaaaaggcctactgtctttaagtacaccgcttgcagtgtttcagtggacattttcacaattctggccgcttggacttcacagtaaccagttaaaactgtggaatatcacttctggttgaaaacccagaggaaaggccctgctgttttccacctaccacgttgtctgatttcataaaagggctgtgggggtgggaagggcagtgggttcggtggtgtgggaaagaaagacgaatggcaggcttcttccccagattctgcccgggtccacacaccctggcccaccttctccatatccccctcttgcagcagaagccaggaagacttggacaagcaacaagcaacagtggctatcgtatttattcagtgtcttcgctgagccacagcctcagcacaatcaagagggactttcatgaaaggcaggaatgcagataaaacaaagatatcagaaatttgcacctatgtttctaggtacaagagaaggattatttccaacaatctttgcaaaaaaaaaaaaa

SEQ ID NO: 8 - DNA, 3290 bp, Homo sapiens
caccttctgcactgctcatctgggcagaggaagcttcagaaagctgccaaggcaccatctccaggaactcccagcacgcagaatccatctgagaatatgctgccacaaataccctttttgctgctagtatccttgaacttggttcatggagtgttttacgctgaacgataccaaatgcccacaggcataaaaggcccactacccaacaccaagacacagttcttcattccctacaccataaagagtaaaggtatagcagtaagaggagagcaaggtactcctggtccaccaggccctgctggacctcgagggcacccaggtccttctggaccaccaggaaaaccaggctacggaagtcctggactccaaggagagccagggttgccaggaccaccgggaccatcagctgtagggaaaccaggtgtgccaggactcccaggaaaaccaggagagagaggaccatatggaccaaaaggagatgttggaccagctggcctaccaggaccccggggcccaccaggaccacctggaatccctggaccggctggaatttctgtgccaggaaaacctggacaacagggacccacaggagccccaggacccaggggctttcctggagaaaagggtgcaccaggagtccctggtatgaatggacagaaaggggaaatgggatatggtgctcctggtcgtccaggtgagaggggtcttccaggccctcagggtcccacaggaccatctggccctcctggagtgggaaaaagaggtgaaaatggggttccaggacagccaggcatcaaaggtgatagaggttttccgggagaaatgggaccaattggcccaccaggtccccaaggccctcctggggaacgagggccagaaggcattggaaagccaggagctgctggagccccaggccagccagggattccaggaacaaaaggtctccctggggctccaggaatagctgggcccccagggcctcctggctttgggaaaccaggcttgccaggcctgaagggagaaagaggacctgctggccttcctgggggtccaggtgccaaaggggaacaagggccagcaggtcttcctgggaagccaggtctgactggaccccctgggaatatgggaccccaaggaccaaaaggcatcccgggtagccatggtctcccaggccctaaaggtgagacagggccagctgggcctgcaggataccctggggctaagggtgaaaggggttcccctgggtcagatggaaaaccagggtacccaggaaaaccaggtctcgatggtcctaagggtaacccagggttaccaggtccaaaaggtgatcctggagttggaggacctcctggtctcccaggccctgtgggcccagcaggagcaaagggaatgcccggacacaatggagaggctggcccaagaggtgcccctggaataccaggtactagaggccctattgggccaccaggcattccaggattccctgggtctaaaggggatccaggaagtcccggtcctcctggcccagctggcatagcaactaagggcctcaatggacccaccgggccaccagggcctccaggtccaagaggccactctggagagcctggtcttccagggccccctgggcctccaggcccaccaggtcaagcagtcatgcctgagggttttataaaggcaggccaaaggcccagtctttctgggacccctcttgttagtgccaaccagggggtaacaggaatgcctgtgtctgcttttactgttattctctccaaagcttacccagcaataggaactcccataccatttgataaaattttgtataacaggcaacagcattatgacccaaggactggaatctttacttgtcagataccaggaatatactatttttcataccacgtgcatgtgaaagggactcatgtttgggtaggcctgtataagaatggcacccctgtaatgtacacctatgatgaatacaccaaaggctacctggatcaggcttcagggagtgccatcatcgatctcacagaaaatgaccaggtgtggctccagcttcccaatgccgagtcaaatggcctatactcctctgagtatgtccactcctctttctcaggattcctagtggctccaatgtgagtacacacagagctaatctaaatcttgtgctagaaaaagcattctctaactctaccccaccctacaaaatgcatatggaggtaggctgaaaagaatgtaatttttattttctgaaatacagatttgagctatcagaccaacaaaccttccccctgaaaagtgagcagcaacgtaaaaacgtatgtgaagcctctcttgaatttctagttagcaatcttaaggctctttaaggttttctccaatattaaaaaatatcaccaaagaagtcctgctatgttaaaaacaaacaacaaaaaacaaacaacaaaaaaaaaattaaaaaaaaaaacagaaatagagctctaagttatgtgaaatttgatttgagaaactcggcatttcctttttaaaaaagcctgtttctaactatgaatatgagaacttctaggaaacatccaggaggtatcatataactttgtagaacttaaatacttgaatattcaaatttaaaagacactgtatcccctaaaatatttctgatggtgcactactctgaggcctgtatggcccctttcatcaatatctattcaaatatacaggtgcatatatacttgttaaagctcttatataaaaaagccccaaaatattgaagttcatctgaaatgcaaggtgctttcatcaatgaaccttttcaaacttttctatgattgcagagaagctttttatatacccagcataacttggaaacaggtatctgacctattcttatttagttaacacaagtgtgattaatttgatttctttaattccttattgaatcttatgtgatatgattttctggatttacagaacattagcacatgtaccttgtgcctcccattcaagtgaagttataatttacactgagggtttcaaaattcgactagaagtggagatatattatttatttatgcactgtactgtatttttatattgctgtttaaaacttttaagctgtgcctcacttattaaagcacaaaatgttttacctactccttatttacgacgcaataaaataacatcaatagatttttaggctgaattaatttgaaagcagcaatttgctgttctcaaccattctttcaaggcttttcattgttcaaagttaataaaaaagtaggacaataaagtga

SEQ ID NO: 9 - protein, 264 aa, Homo sapiens
MIMSSYLMDSNYIDPKFPPCEEYSQNSYIPEHSPEYYGRTRESGFQHHHQELYPPPPPRPSYPERQYSCTSLQGPGNSRGHGPAQAGHHHPEKSQSLCEPAPLSGASASPSPAPPACSQPAPDHPSSAASKQPIVYPWMKKIHVSTVNPNYNGGEPKRSRTAYTRQQVLELEKEFHYNRYLTRRRRIEIAHSLCLSERQIKIWFQNRRMKWKKDHRLPNTKVRSAPPAGAAPSTLSAATPGTSEDHSQSATPPEQQRAEDITRL

SEQ ID NO: 10 - DNA, 2459 bp, Homo sapiens
acacacgtgttacatggatacagctcctagccagaagcaagcaggctctacccacacaggcctccctcttagctagcgggggctcaacccccagacctccagaaatgacgtcagaatcatttgcatcccgctgcctctacctgcctggtccagctgggaccctgcctcgccggccgcatggccagagggttgggtgagtgtgtatggggaagaggggctggactctggtatccttggatggggggcactccaggctctccagcctcctcggctcagcctgggcccctccccatccaacatccactccagtcctcattcaacttcctcttcctgcgaaagaggggcgctgccccgtgacctacacagactgagacacgatcgccatgaatggagacctctggaaaagctcaggagccgaggcccacggggcccagcagaggcctgaggggagaccctgggcgggggctgaatcactgcctcccgacagtcccccaatgcccgggctttggaggggagccgggagcttcccatctccttttgcaggggagggttgtcagtctggcgggatgtgcactgggggcactccaacctctgctagctaaccccacatcaccacccacccccgcctcccagcaccaccaccaccacacacacaaaaaaattggatacattttgaataaagcgattcggttccttatccggggactgggttgctccgtgtgattggccggaggagtcacatggtgaaagtaactttacagggtcgctagctagtaggagggctttatggagcagaaaaacgacaaagcgagaaaaattattttccactccagaaattaatgatcatgagctcgtatttgatggactctaactacatcgatccgaaatttcctccatgcgaagaatattcgcaaaatagctacatccctgaacacagtccggaatattacggccggaccagggaatcgggattccagcatcaccaccaggagctgtacccaccaccgcctccgcgccctagctaccctgagcgccagtatagctgcaccagtctccaggggcccggcaattcgcgaggccacgggccggcccaggcgggccaccaccaccccgagaaatcacagtcgctctgcgagccggcgcctctctcaggcgcctccgcctccccgtccccagccccgccagcctgcagccagccagcccccgaccatccctccagcgccgccagcaagcaacccatagtctacccatggatgaaaaaaattcacgttagcacggtgaaccccaattataacggaggggaacccaagcgctcgaggacagcctatacccggcagcaagtcctggaattagagaaagagtttcattacaaccgctacctgacccgaaggagaaggatcgagatcgcccactcgctgtgcctctctgagaggcagatcaaaatctggttccaaaaccgtcgcatgaaatggaagaaggaccaccgactccccaacaccaaagtcaggtcagcacccccggccggcgctgcgcccagcaccctttcggcagctaccccgggtacttctgaagaccactcccagagcgccacgccgccggagcagcaacgggcagaggacattaccaggttataaaacataactcacacccctgcccccaccccatgcccccaccctcccctcacacacaaattgactcttatttatagaatttaatatatatatatatatatatatatataggttcttttctctcttcctctcaccttgtcccttgtcagttccaaacagacaaaacagataaacaaacaagccccctgccctcctctccctcccactgttaaggacccttttaagcatgtgatgttgtcttagcatggtacctgctgggtgtttttttttaaaaggccattttggggggttatttattttttaagaaaaaaagctgcaaaaattatatattgcaaggtgtgatggtctggcttgggtgaatttcaggggaaatgaggaaaagaaaaaaggaaagaaattttaaagccaattctcatccttctcctcctcctccttccccccctctttccttaggccttttgcattgaaaatgcaccaggggaggttagtgagggggaagtcattttaaggagaacaaagctatgaagttcttttgtattattgttgggggggggtgtgggaggagagggggcgaagacagcagacaaagctaaatgcatctggagagcctctcagagctgttcagtttgaggagccaaaagaaaatcaaaatgaactttcagttcagagaggcagtctataggtagaatctctccccacccctatcgtggttattgtgtttttggactgaatttacttgattattgtaaaacttgcaataaagaattttagtgtcgatgtgaaatgccccgtgatcaataataaaccagtggatgtgaattagtttta

SEQ ID NO: 11 - protein, 2120 aa, Homo sapiens
MRSFKRVNFGTLLSSQKEAEELLPALKEFLSNPPAGFPSSRSDAERRQACDAILRACNQQLTAKLACPRHLGSLLELAELACDGYLVSTPQRPPLYLERILFVLLRNAAAQGSPEATLRLAQPLHACLVQCSREAAPQDYEAVARGSFSLLWKGAEALLERRAAFAARLKALSFLVLLEDESTPCEVPHFASPTACRAVAAHQLFDASGHGLNEADADFLDDLLSRHVIRALVGERGSSSGLLSPQRALCLLELTLEHCRRFCWSRHHDKAISAVEKAHSYLRNTNLAPSLQLCQLGVKLLQVGEEGPQAVAKLLIKASAVLSKSMEAPSPPLRALYESCQFFLSGLERGTKRRYRLDAILSLFAFLGGYCSLLQQLRDDGVYGGSSKQQQSFLQMYFQGLHLYTVVVYDFAQGCQIVDLADLTQLVDSCKSTVVWMLEALEGLSGQELTDHMGMTASYTSNLAYSFYSHKLYAEACAISEPLCQHLGLVKPGTYPEVPPEKLHRCFRLQVESLKKLGKQAQGCKMVILWLAALQPCSPEHMAEPVTFWVRVKMDAARAGDKELQLKTLRDSLSGWDPETLALLLREELQAYKAVRADTGQERFNIICDLLELSPEETPAGAWARATHLVELAQVLCYHDFTQQTNCSALDAIREALQLLDSVRPEAQARDQLLDDKAQALLWLYICTLEAKMQEGIERDRRAQAPGNLEEFEVNDLNYEDKLQEDRFLYSNIAFNLAADAAQSKCLDQALALWKELLTKGQAPAVRCLQQTAASLQILAALYQLVAKPMQALEVLLLLRIVSERLKDHSKAAGSSCHITQLLLTLGCPSYAQLHLEEAASSLKHLDQTTDTYLLLSLTCDLLRSQLYWTHQKVTKGVSLLLSVLRDPALQKSSKAWYLLRVQVLQLVAAYLSLPSNNLSHSLWEQLCAQGWQTPEIALIDSHKLLRSIILLLMGSDILSTQKAAVETSFLDYGENLVQKWQVLSEVLSCSEKLVCHLGRLGSVSEAKAFCLEALKLTTKLQIPRQCALFLVLKGELELARNDIDLCQSDLQQVLFLLESCTEFGGVTQHLDSVKKVHLQKGKQQAQVPCPPQLPEEELFLRGPALELVATVAKEPGPIAPSTNSSPVLKTKPQPIPNFLSHSPTCDCSLCASPVLTAVCLRWVLVTAGVRLAMGHQAQGLDLLQVVLKGCPEAAERLTQALQASLNHKTPPSLVPSLLDEILAQAYTLLALEGLNQPSNESLQKVLQSGLKFVAARIPHLEPWRASLLLIWALTKLGGLSCCTTQLFASSWGWQPPLIKSVPGSEPSKTQGQKRSGRGRQKLASAPLRLNNTSQKGLEGRGLPCTPKPPDRIRQAGPHVPFTVFEEVCPTESKPEVPQAPRVQQRVQTRLKVNFSDDSDLEDPVSAEAWLAEEPKRRGTASRGRGRARKGLSLKTDAVVAPGSAPGNPGLNGRSRRAKKVASRHCEERRPQRASDQARPGPEIMRTIPEEELTDNWRKMSFEILRGSDGEDSASGGKTPAPGPEAASGEWELLRLDSSKKKLPSPCPDKESDKDLGPRLRLPSAPVATGLSTLDSICDSLSVAFRGISHCPPSGLYAHLCRFLALCLGHRDPYATAFLVTESVSITCRHQLLTHLHRQLSKAQKHRGSLEIADQLQGLSLQEMPGDVPLARIQRLFSFRALESGHFPQPEKESFQERLALIPSGVTVCVLALATLQPGTVGNTLLLTRLEKDSPPVSVQIPTGQNKLHLRSVLNEFDAIQKAQKENSSCTDKREWWTGRLALDHRMEVLIASLEKSVLGCWKGLLLPSSEEPGPAQEASRLQELLQDCGWKYPDRTLLKIMLSGAGALTPQDIQALAYGLCPTQPERAQELLNEAVGRLQGLTVPSNSHLVLVLDKDLQKLPWESMPSLQALPVTRLPSFRFLLSYSIIKEYGASPVLSQGVDPRSTFYVLNPHNNLSSTEEQFRANFSSEAGWRGVVGEVPRPEQVQEALTKHDLYIYAGHGAGARFLDGQAVLRLSCRAVALLFGCSSAALAVRGNLEGAGIVLKYIMAGCPLFLGNLWDVTDRDIDRYTEALLQGWLGAGPGAPLLYYVNQARQAPRLKYLIGAAPIAYGLPVSLR

SEQ ID NO: 12 - DNA, 6641 bp, Homo sapiens
ggttacattttggatcctcgcggagtactggtcaggcggttaagtcctgtacctaggaaagagggcgagctctggggcgctctccggtgtcatgaggagcttcaaaagagtcaactttgggactctgctaagcagccagaaggaggctgaagagttgctgcccgccttgaaggagttcctgtccaaccctccagctggttttcccagcagccgatctgatgctgagaggagacaagcttgtgatgccatcctgagggcttgcaaccagcagctgactgctaagctagcttgccctaggcatctggggagcctgctggagctggcagagctggcctgtgatggctacttagtgtctaccccacagcgtcctcccctctacctggaacgaattctctttgtcttactgcggaatgctgctgcacaaggaagcccagaggccacactccgccttgctcagcccctccatgcctgcttggtgcagtgctctcgcgaggctgctccccaggactatgaggccgtggctcggggcagcttttctctgctttggaagggggcagaagccctgttggaacggcgagctgcatttgcagctcggctgaaggccttgagcttcctagtactcttggaggatgaaagtaccccttgtgaggttcctcactttgcttctccaacagcctgtcgagcggtagctgcccatcagctatttgatgccagtggccatggtctaaatgaagcagatgctgatttcctagatgacctgctctccaggcacgtgatcagagccttggtgggtgagagagggagctcttctgggcttctttctccccagagggccctctgcctcttggagctcaccttggaacactgccgtcgcttttgctggagccgccaccatgacaaagccatcagcgcagtggagaaggctcacagttacctaaggaacaccaatctagcccctagccttcagctatgtcagctgggggttaagctgctgcaggttggggaggaaggacctcaggcagtggccaagcttctgatcaaggcatcagctgtcctgagcaagagtatggaggcaccatcacccccacttcgggcattgtatgagagctgccagttcttcctttcaggcctggaacgaggcaccaagaggcgctatagacttgatgccattctgagcctctttgcttttcttggagggtactgctctcttctgcagcagctgcgggatgatggtgtgtatgggggctcctccaagcaacagcagtcttttcttcagatgtactttcagggacttcacctctacactgtggtggtttatgactttgcccaaggctgtcagatagttgatttggctgacctgacccaactagtggacagttgtaaatctaccgttgtctggatgctggaggccttagagggcctgtcgggccaagagctgacggaccacatggggatgaccgcttcttacaccagtaatttggcctacagcttctatagtcacaagctctatgccgaggcctgtgccatctctgagccgctctgtcagcacctgggtttggtgaagccaggcacttatcccgaggtgcctcctgagaagttgcacaggtgcttccggctacaagtagagagtttgaagaaactgggtaaacaggcccagggctgcaagatggtgattttgtggctggcagccctgcaaccctgtagccctgaacacatggctgagccagtcactttctgggttcgggtcaagatggatgcggccagggctggagacaaggagctacagctaaagactctgcgagacagcctcagtggctgggacccggagaccctggccctcctgctgagggaggagctgcaggcctacaaggcggtgcgggccgacactggacaggaacgcttcaacatcatctgtgacctcctggagctgagccccgaggagacaccagccggggcctgggcacgagccacccacctggtagaactggctcaggtgctctgctaccacgactttacgcagcagaccaactgctctgctctggatgctatccgggaagccctgcagcttctggactctgtgaggcctgaggcccaggccagagatcagcttctggacgataaagcacaggccttgctgtggctttacatctgtactctggaagccaaaatgcaggaaggtatcgagcgggatcggagagcccaggcccctggtaacttggaggaatttgaagtcaatgacctgaactatgaagataaactccaggaagatcgtttcctatacagtaacattgccttcaacctggctgcagatgctgctcagtccaaatgcctggaccaagccctggccctgtggaaggagctgcttacaaaggggcaggccccagctgtacggtgtctccagcagacagcagcctcactgcagatcctagcagccctctaccagctggtggcaaagcccatgcaggctctggaggtcctcctgctgctacggattgtctctgagagactgaaggaccactcgaaggcagctggctcctcctgccacatcacccagctcctcctgaccctcggctgtcccagctatgcccagttacacctggaagaggcagcatcgagcctgaagcatctcgatcagactactgacacatacctgctcctttccctgacctgtgatctgcttcgaagtcaactctactggactcaccagaaggtgaccaagggtgtctctctgctgctgtctgtgcttcgggatcctgccctccagaagtcctccaaggcttggtacttgctgcgtgtccaggtcctgcagctggtggcagcttaccttagcctcccgtcaaacaacctctcacactccctgtgggagcagctctgtgcccaaggctggcagacacctgagatagctctcatagactcccataagctcctccgaagcatcatcctcctgctgatgggcagtgacattctctcaactcagaaagcagctgtggagacatcgtttttggactatggtgaaaatctggtacaaaaatggcaggttctttcagaggtgctgagctgctcagagaagctggtctgccacctgggccgcctgggtagtgtgagtgaagccaaggccttttgcttggaggccctaaaacttacaacaaagctgcagataccacgccagtgtgccctgttcctggtgctgaagggcgagctggagctggcccgcaatgacattgatctctgtcagtcggacctgcagcaggttctgttcttgcttgagtcttgcacagagtttggtggggtgactcagcacctggactctgtgaagaaggtccacctgcagaaggggaagcagcaggcccaggtcccctgtcctccacagctcccagaggaggagctcttcctaagaggccctgctctagagctggtggccactgtggccaaggagcctggccccatagcaccttctacaaactcctccccagtcttgaaaaccaagccccagcccatacccaacttcctgtcccattcacccacctgtgactgctcgctctgcgccagccctgtcctcacagcagtctgtctgcgctgggtattggtcacggcaggggtgaggctggccatgggccaccaagcccagggtctggatctgctgcaggtcgtgctgaagggctgtcctgaagccgctgagcgcctcacccaagctctccaagcttccctgaatcataaaacacccccctccttggttccaagcctcttggatgagatcttggctcaagcatacacactgttggcactggagggcctgaaccagccatcaaacgagagcctgcagaaggttctacagtcagggctgaagtttgtagcagcacggataccccacctagagccctggcgagccagcctgctcttgatttgggccctcacaaaactaggtggcctcagctgctgtactacccaactttttgcaagctcctggggctggcagccaccattaataaaaagtgtccctggctcagagccctctaagactcagggccaaaaacgttctggacgagggcgccaaaagttagcctctgctcccctgcgcctcaataatacctctcagaaaggtctggaaggtagaggactgccctgcacacctaaacccccagaccggatcaggcaagctggccctcatgtccccttcacggtgtttgaggaagtctgccctacagagagcaagcctgaagtaccccaggcccccagggtacaacagagagtccagacgcgcctcaaggtgaacttcagtgatgacagtgacttggaagaccctgtctcagctgaggcctggctggcagaggagcctaagagacggggcactgcttcccggggccgggggcgagcaaggaagggcctgagcctaaagacggatgccgtggttgccccaggtagtgcccctgggaaccctggcctgaatggcaggagccggagggccaagaaggtggcatcaagacattgtgaggagcggcgtccccagagggccagtgaccaggccaggcctggccctgagatcatgaggaccatccctgaggaagaactgactgacaactggagaaaaatgagctttgagatcctcaggggctctgacggggaagactcagcctcaggtgggaagactccagctccgggccctgaggcagcttctggagaatgggagctgctgaggctggattccagcaagaagaagctgcccagcccatgcccagacaaggagagtgacaaggaccttggtcctcggctccggctcccctcagcccccgtagccactggtctttctaccctggactccatctgtgactccctgagtgttgctttccggggcattagtcactgtcctcctagtgggctctatgcccacctctgccgcttcctggccttgtgcctgggccaccgggatccttatgccactgctttccttgtcaccgagtctgtctccatcacctgtcgccaccagctgctcacccacctccacagacagctcagcaaggcccagaagcaccgaggatcacttgaaatagcagaccagctgcaggggctgagccttcaggagatgcctggagatgtccccctggcccgcatccagcgcctcttttccttcagggctttggaatctggccacttcccccagcctgaaaaggagagtttccaggagcgcctggctctgatccccagtggggtgactgtgtgtgtgttggccctggccaccctccagcccggaaccgtgggcaacaccctcctgctgacccggctggaaaaggacagtcccccagtcagtgtgcagattcccactggccagaacaagcttcatctgcgttcagtcctgaatgagtttgatgccatccagaaggcacagaaagagaacagcagctgtactgacaagcgagaatggtggacagggcggctggcactggaccacaggatggaggttctcatcgcttccctagagaagtctgtgctgggctgctggaaggggctgctgctgccgtccagtgaggagcccggccctgcccaggaggcctcccgcctacaggagctgctacaggactgtggctggaaatatcctgaccgcactctgctgaaaatcatgctcagtggtgccggtgccctcacccctcaggacattcaggccctggcctacgggctgtgcccaacccagccagagcgagcccaggagctcctgaatgaggcagtaggacgtctacagggcctgacagtaccaagcaatagccaccttgtcttggtcctagacaaggacttgcagaagctgccgtgggaaagcatgcccagcctccaagcactgcctgtcacccggctgccctccttccgcttcctactcagctactccatcatcaaagagtatggggcctcgccagtgctgagtcaaggggtggatccacgaagtaccttctatgtcctgaaccctcacaataacctgtcaagcacagaggagcaatttcgagccaatttcagcagtgaagctggctggagaggagtggttggggaggtgccaagacctgaacaggtgcaggaagccctgacaaagcatgatttgtatatctatgcagggcatggggctggtgcccgcttccttgatgggcaggctgtcctgcggctgagctgtcgggcagtggccctgctgtttggctgtagcagtgcggccctggctgtgcgtggaaacctggagggggctggcatcgtgctcaagtacatcatggctggttgccccttgtttctgggtaatctctgggatgtgactgaccgcgacattgaccgctacacggaagctctgctgcaaggctggcttggagcaggcccaggggccccccttctctactatgtaaaccaggcccgccaagctccccgactcaagtatcttattggggctgcacctatagcctatggcttgcctgtctctctgcggtaaccccatggagctgtcttattgatgctagaagcctcataactgttctacctccaaggttagatttaatccttaggataactcttttaaagtgattttccccagtgttttatatgaaacatttccttttgatttaacctcagtataataaagatacatcatttaaaccctgaaaaaaaaaaaaaaaaaa

SEQ ID NO: 13 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 14 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 15 - protein, 5058 aa, Homo sapiens
  MOD_RES (283)..(283): Any amino acid
  MOD_RES (2674)..(2674): Any amino acid
MGHAGCQFKALLWKNWLCRLRNPVLFLAEFFWPCILFVILTVLRFQEPPRYRDICYLQPRDLPSCGVIPFVQSLLCNTGSRCRNFSYEGSMEHHFRLSRFQTAADPKKVNNLAFLKEIQDLAEEIHGMMDKAKNLKRLWVERSNTPDSSYGSSFFTMDLNKTEEVILKLESLHQQPHIWDFLLLLPRLHTSHDHVEDGMDVAVNLLQTILNSLISLEDLDWLPLNQTFSQVSELVLNVTISTLTFLQQHGVAVTEPVYHLSMQNIVWDPQKVQYDLKSQFGFXDLHTEQILNSSAELKEIPTDTSLEKMVCSVLSSTSEDEAEKWGHVGGCHPKWSEAKNYLVHAVSWLRVYQQVFVQWQQGSLLQKTLTGMGHSLEALRNQFEEESKPWKVVEALHTALLLLNDSLSADGPKDNHTFPKILQHLWKLQSLLQNLPQWPALKRFLQLDGALRNAIAQNLHFVQEVLICLETSANDFKWFELNQLKLEKDVFFWELKQMLAKNAVCPNGRFSEKEVFLPPGNSSIWGGLQGLLCYCNSSETSVLNKLLGSVEDADRILQEVITWHKNMSVLIPEEYLDWQELEMQLSEASLSCTRLFLLLGADPSPENDVFSSDCKHQLVSTVIFHTLEKTQFFLEQAYYWKAFKKFIRKTCEVAQYVNMQESFQNRLLAFPEESPCFEENMDWKMISDNYFQFLNNLLKSPTASISRALNFTKHLLMMEKKLHTLEDEQMNFLLSFVEFFEKLLLPNLFDSSIVPSFHSLPSLTEDILNISSLWTNHLKSLKRDPSATDAQKLLEFGNEVIWKMQTLGSHWIRKEPKNLLRFIELILFEINPKLLELWAYGISKGKRAKLENFFTLLNFSVPENEILSTSFNFSQLFHSDWPKSPAMNIDFVRLSEAIITSLHEFGFLEQEQISEALNTVYAIRNASDLFSALSEPQKQEVDKILTHIHLNVFQDKDSALLLQIYSSFYRYIYELLNIQSRGSSLTFLTQISKHILDIIKQFNFQNISKAFAFLFKTAEVLGGISNVSYCQQLLSIFNFLELQAQSFMSTEGQELEVIHTTLTGLKQLLIIDEDFRISLFQYMSQFFNSSVEDLLDNKCLISDNKHISSVNYSTSEESSFVFPLAQIFSNLSANVSVFNKFMSIHCTVSWLQMWTEIWETISQLFKFDMNVFTSLHHGFTQLLDELEDDVKVSKSCQGILPTHNVARLILNLFKNVTQANDFHNWEDFLDLRDFLVALGNALVSVKKLNLEQVEKSLFTMEAALHQLKTFPFNESTSREFLNSLLEVFIEFSSTSEYIVRNLDSINDFLSNNLTNYGEKFENIITELREAIVFLRNVSHDRDLFSCADIFQNVTECILEDGFLYVNTSQRMLRILDTLNSTFSSENTISSLKGCIVWLDVINHLYLLSNSSFSQGRLQNILGNFRDIENKMNSILKIVTWVLNIKKPLCSSNGSHINCVNIYLKDVTDFLNIVLTTVFEKEKKPKFEILLALLNDSTKQVRMSINNLTTDFDFASQSNWRYFTELILRPIEMSDEIPNQFQNIWLHLITLGKEFQKLVKGIYFNILENNSSSKTENLLNIFATSPKEKDVNSVGNSIYHLASYLAFSLSHDLQNSPKIIISPEIMKATGLGIQLIRDVFNSLMPVVHHTSPQNAGYMQALKKVTSVMRTLKKADIDLLVDQLEQVSVNLMDFFKNISSVGTGNLVVNLLVGLMEKFADSSHSWNVNHLLQLSRLFPKDVVDAVIDVYYVLPHAVRLLQGVPGKNITEGLKDVYSFTLLHGITISNITKEDFAIVIKILLDTIELVSDKPDIISEALACFPVVWCWNHTNSGFRQNSKIDPCNVHGLMSSSFYGKVASILDHFHLSPQGEDSPCSNESSRMEITRKVVCIIHELVDWNSILLELSEVFHVNISLVKTVQKFWHKILPFVPPSINQTRDSISELCPSGSIKQVALQIIEKLKNVNFTKVTSGENILDKLSSLNKILNINEDTETSVQNIISSNLERTVQLISEDWSLEKSTHNLLSLFMMLQNANVTGSSLEALSSFIEKSETPYNFEELWPKFQQIMKDLTQDFRIRHLLSEMNKGIKSINSMALQKITLQFAHFLEILDSPSLKTLEIIEDFLLVTKNWLQEYANEDYSRMIETLFIPVTNESSTEDIALLAKAIATFWGSLKNISRAGNFDVAFLTHLLNQEQLTNFSVVQLLFENILINLINNLAGNSQEAAWNLNDTDLQIMNFINLILNHMQSETSRKTVLSLRSIVDFTEQFLKTFFSLFLKEDSENKISLLLKYFHKDVIAEMSFVPKDKILEILKLDQFLTLMIQDRLMNIFSSLKETIYHLMKSSFILDNGEFYFDTHQGLKFMQDLFNALLRETSMKNKTENNIDFFTVVSQLFFHVNKSEDLFKLNQDLGSALHLVRECSTEMARLLDTILHSPNKDFYALYPTLQEVILANLTDLLFFINNSFPLRNRATLEITKRLVGAISRASEESHVLKPLLEMSGTLVMLLNDSADLRDLATSMDSIVKLLKLVKKVSGKMSTVFKTHFISNTKDSVKFFDTLYSIMQQSVQNLVKEIATLKKIDHFTFEKINDLLVPFLDLAFEMIGVEPYISSNSDIFSMSPSILSYMNQSKDFSDILEEIAEFLTSVKMNLEDMRSLAVAFNNETQTFSMDSVNLXEEILGCLVPINNITNQMDFLYPNPISTHSGPQDIKWEIIHEVILFLDKILSQNSTEIGSFLKMVICLTLEALWKNLKKDNWNVSNVLMTFTQHPNNLLKTIETVLEASSGIKSDYEGDLNKSLYFDTPLSQNITHHQLEKAIHNVLSRIALWRKGLRFNNSEWITSTRTLFQPLFEIFIKATTGKNVTSEKEERTEKEMIDFPYSFKPFFCLEKYLGGLFVLTKYWQQIPLTDQSVVEICEVFQQTVKPSEAMEMLQKVKMMVVRVLTIVAENPSWTKDILCATLSCKQNGIRHLILSAIQGVTLAQDHFQEIEKIWSSPNQLNCESLSKNLSSTLESFKSSLENATGQDCTSQPRLETVQQHLYMLAKSLEETWSSGNPIMTFLSNFTVTEDVKIKDLMKNITKLTEELRSSIQISNETIHSILEANISHSKVLFSALTVALSGKCDQEILHLLLTFPKGEKSWIAAEELCSLPGSKVYSLIVLLSRNLDVRAFIYKTLMPSEANGLLNSLLDIVSSLSALLAKAQHVFEYLPEFLHTFKITALLETLDFQQVSQNVQARSSAFGSFQFVMKMVCKDQASFLSDSNMFINLPRVKELLEDDKEKFNIPEDSTPFCLKLYQEILQLPNGALVWTFLKPILHGKILYTPNTPEINKVIQKANYTFYIVDKLKTLSETLLEMSSLFQRSGSGQMFNQLQEALRNKFVRNFVENQLHIDVDKLTEKLQTYGGLLDEMFNHAGAGRFRFLGSILVNLSSCVALNRFQALQSVDILETKAHELLQQNSFLASIIFSNSLFDKNFRSESVKLPPHVSYTIRTNVLYSVRTDVVKNPSWKFHPQNLPADGFKYNYVFAPLQDMIERAIILVQTGQEALEPAAQTQAAPYPCHTSDLFLNNVGFFFPLIMMLTWMVSVASMVRKLVYEQEIQIEEYMRMMGVHPVIHFLAWFLENMAVLTISSATLAIVLKTSGIFAHSNTFIVFLFLLDFGMSVVMLSYLLSAFFSQANTAALCTSLVYMISFLPYIVLLVLHNQLSFVNQTFLCLLSTTAFGQGVFFITFLEGQETGIQWNNMYQALEQGGMTFGWVCWMILFDSSLYFLCGWYLSNLIPGTFGLRKPWYFPFTASYWKSVGFLVEKRQYFLSSSLFFFNENFDNKGSSLQNREGELEGSAPGVTLVSVTKEYEGHKAVVQDLSLTFYRDQITALLGTNGAGKTTIISMLTGLHPPTSGTIIINGKNLQTDLSRVRMELGVCPQQDILLDNLTVREHLLLFASIKAPQWTKKELHQQVNQTLQDVDLTQHQHKQTRALSGGLKRKLSLGIAFMGMSRTVVLDEPTSGVDPCSRHSLWDILLKYREGRTIIFTTHHLDEAEALSDRVAVLQHGRLRCCGPPFCLKEAYGQGLRLTLTRQPSVLEAHDLKDMACVTSLIKIYIPQAFLKDSSGSELTYTIPKDTDKACLKGLFQALDENLHQLHLTGYGISDTTLEEVFLMLLQDSNKKSHIALGTESELQNHRPTGHLSGYCGSLARPATVQGVQLLRAQVAAILARRLRRTLRAGKSTLADLLLPVLFVALAMGLFMVRPLATEYPPLRLTPGHYQRAETYFFSSGGDNLDLTRVLLRKFRDQDLPCADLNPRQKNSSCWRTDPFSHPEFQDSCGCLKCPNRSASAPYLTNHLGHTLLNLSGFNMEEYLLAPSEKPRLGGWSFGLKIPSEAGGANGNISKPPTLAKVWYNQKGFHSLPSYLNHLNNLILWQHLPPTVDWRQYGITLYSHPYGGALLNEDKILESIRQCGVALCIVLGFSILSASIGSSVVRDRVIGAKRLQHISGLGYRMYWFTNFLYDMLFYLVSVCLCVAVIVAFQLTAFTFRKNLAATALLLSLFGYATLPWMYLMSRIFSSSDVAFISYVSLNFIFGLCTMPITIMPRLLAIISKAKNLQNIYDVLKWVFTIFPQFCLGQGLVELCYNQIKYDLTHNFGIDSYVSPFEMNFLGWIFVQLASQGTVLLLLRVLLHWDLLRWPRGHSTLQGTVKSSKDTDVEKEEKRVFEGRTNGDILVLYNLSKHYRRFFQNIIAVQDISLGIPKGECFGLLGVNGAGKSTTFKMLNGEVSLTSGHAIIRTPMGDAVDLSSAGTAGVLIGYCPQQDALDELLTGWEHLYYYCSLRGIPRQCIPEVAGDLIRRLHLEAHADKPVATYSGGTKRKLSTALALVGKPDILLLDEPSSGMDPCSKRYLWQTIMKEVREGCAAVLTSHSMEECEALCTRLAIMVNGSFKCLGSPQHIKNRFGDGYTVKVWLCKEANQHCTVSDHLKLYFPGIQFKGQHLNLLEYHVPKRWGCLADLFKVIENNKTFLNIKHYSINQTTLEQVFINFASEQQQTLQSTLDPSTDSHHTHHLPI

SEQ ID NO: 16 - DNA, 17209 bp, Homo sapiens
ggactgagagcagggagcagcaggcatggggcatgccgggtgccagttcaaagccctgctgtggaagaattggctctgcagactcaggaacccggtccttttccttgctgaattcttctggccttgtatcctgtttgtaattctgacagttcttcgttttcaagaacctcccagatacagagacatttgttatttgcagccccgagatctacccagctgtggtgttatcccctttgttcaaagccttctttgtaacactggatcaaggtgtaggaacttcagctatgaagggtcaatggagcatcattttcgtttgtctaggttccaaactgcagctgaccccaagaaagtcaacaacctggcctttttaaaagagatacaagacctggcagaggaaattcatggaatgatggacaaggcaaaaaacttaaaaagactttgggtagaacgatccaacactccagattcttcttatggttccagtttttttacaatggatctcaataagaccgaggaggtaatattgaaactggaaagcctccatcagcagcctcatatctgggattttctacttttactgccgagactacacacaagccatgatcatgtggaagatggcatggatgttgcagtgaaccttctccagaccattttgaattccttaatatccctagaagatttagattggcttccactcaaccaaactttttcccaggtttctgaacttgtactgaatgtgaccatttcgacactgacatttctgcagcaacatggagtagcagtcaccgagccagtttaccacctgtccatgcagaatatagtgtgggatccacagaaagtccagtatgatctcaaatcccagtttggctttgrtgatcttcacacggaacagatcctgaactcttcagctgaactgaaggagattcccacagacacttccttggagaagatggtgtgttcagtcttgtctagcacatcagaggatgaagctgagaaatggggccacgttggaggctgccaccctaagtggtcagaagccaaaaactatcttgtccatgcagtcagctggctgcgagtctaccaacaggtgtttgttcagtggcaacagggtagcctgcttcagaagacactcacaggcatgggccatagtctggaggctctcaggaatcagtttgaagaagagagcaagccctggaaggtggtggaagctctgcacactgcactgctcctgctgaatgacagcttgtcagcagatggcccaaaagataatcatacatttccaaagatattacagcatctgtggaaattgcaaagcttgctgcaaaacctgccccagtggccggcactgaagagatttcttcagcttgatggagctctcagaaatgcgatagctcagaatttacattttgtccaagaagtcctcatttgcctggagacatcagctaatgattttaaatggtttgaacttaaccaattgaaactggaaaaggatgtgttcttttgggagctgaaacagatgttggcgaagaatgctgtctgcccgaatggtcgtttctctgagaaggaggtctttttgccgcctggaaactccagcatatggggtggtctccagggactgttgtgctattgtaactcctctgagacgagtgttttaaacaagctacttggttcagtagaggatgctgatcgtattttgcaagaggtcattacttggcacaaaaatatgtcagttttaatacctgaagaatatttggactggcaggaacttgagatgcagctgtcagaagcaagcctttcctgtactcggctcttcctgctgctgggagctgatccctctcctgagaatgatgtcttttctagtgactgtaagcaccagcttgtctccacagtgatatttcatacacttgaaaaaacacaatttttcctggaacaagcatattattggaaagccttcaaaaagtttatcaggaagacttgcgaagtggcccaatatgtaaatatgcaagagagtttccagaacagactattggcttttcctgaggaatctccttgttttgaagaaaacatggattggaaaatgatcagtgataattattttcaatttttgaataacttactcaagtctccaacagcttccatatccagggctttaaatttcacaaagcaccttctaatgatggaaaagaagttgcacacccttgaggatgaacaaatgaactttcttttatcatttgtggaattttttgagaaattattgttgcctaatctttttgactcctccattgttcccagtttccacagcctcccatctctcacagaggatattctgaatataagttctctgtggacaaatcatttaaaaagtttaaagagagacccatctgccactgatgctcagaaactcttggaatttggcaacgaagtgatttggaaaatgcagactctcggaagtcactggataaggaaggaaccaaaaaatcttttgagattcatagaattaatactttttgaaattaatcccaaattactagaattatgggcctatggcatttcaaaaggaaaaagagctaaattggaaaacttctttacacttttaaatttttctgttccagaaaatgagattctgagtacaagttttaacttttcccagttgttccattcagattggcctaaatcaccagctatgaacatagattttgtacgtttaagtgaggctataataactagtctccatgaatttggatttttggagcaggaacagatctcagaagctctgaacacagtctacgctatcaggaatgcatctgatcttttctcagccctttctgaaccacaaaaacaagaagttgataaaattttgactcacatacacctaaatgtcttccaggacaaggattcagctttacttctgcaaatttattcttcattttaccgatatatttatgaattattgaatattcagagtagaggctcttcgttgactttccttacacaaatctcaaaacacattttggatatcataaaacaatttaatttccaaaacatcagtaaagcatttgcatttttatttaagacagcagaggttcttgggggaatttctaatgtatcttactgtcagcaattgctttcaatttttaactttttggagcttcaggcccaatccttcatgtctacagagggccaagaactggaagtgatccacactactttgacaggcctcaaacagctgctcataattgatgaagattttcgtatttctttatttcaatatatgagccaattcttcaacagttcagtagaagacctattggataataaatgcttgatttcggacaataaacacatttcttccgtaaattattcaacaagtgaggagtcttcatttgtttttccattggcacaaattttttcaaacctctcagcaaatgtcagtgtgttcaacaagtttatgtccattcactgtaccgtttcatggcttcaaatgtggactgaaatctgggaaaccatatctcaattatttaagtttgacatgaatgttttcacatctcttcatcatggtttcactcagcttttggatgaattggaagatgatgtgaaagtctctaaaagctgccagggtatacttcccacccataatgttgctagactcatattaaatttgtttaaaaatgtaactcaagccaatgacttccataattgggaggacttcctggatctcagggattttttggtagctttaggtaatgcattagtttcagtaaaaaaacttaacttggagcaagtggagaaatcccttttcaccatggaagctgccctgcatcagttgaagacatttccattcaacgaaagtacaagcagagagtttttaaattctctgcttgaagttttcattgagtttagcagtacctcagaatatatagtcagaaatctagattcaataaatgactttctttcaaataatctcacaaattatggagaaaaatttgaaaatatcatcactgagctaagagaagcaatagtatttcttagaaatgtatcacatgatcgagatttgttttcctgtgctgatattttccaaaatgttactgagtgtattttagaagatggctttttatatgtaaatacctcacagaggatgttacgtattctagacacgttaaattccacattttcctctgagaacacaattagcagtctgaaaggatgcattgtatggttagatgtcataaaccatttgtatttgttgtctaactccagtttttcacaaggtcgtcttcaaaatattttggggaatttcagagatatagaaaacaaaatgaactctatattaaaaattgtaacttgggtgttaaatataaaaaaacctctttgttcatcaaatggctcacatataaattgtgtcaatatttacttgaaagatgtaactgactttctaaatattgtacttactacagtctttgaaaaagagaagaaacctaaatttgagattttattagctcttttaaatgattccacaaagcaagtaaggatgagtatcaacaacttaacaacagactttgattttgcatctcagtccaattggagatattttactgaattaattctaagaccaatagaaatgtcagatgaaattcctaatcagtttcaaaatatttggcttcatttaataacactggggaaggaatttcagaagcttgtaaaaggtatttattttaacatcctggaaaataattcctcttctaaaactgaaaacttgttaaacatatttgccaccagtccaaaagaaaaggatgtaaacagtgtaggcaattccatttatcacttagctagttaccttgccttcagcttatctcatgacctccaaaattcaccaaaaataataatttcacctgaaataatgaaagctacaggtcttggtattcaactgataagggatgtgttcaactccttaatgcctgtagttcatcacactagtccacaaaatgcaggttatatgcaagctttgaagaaggtaacttctgtcatgcgtacccttaagaaagcagacatagaccttttagtggatcagcttgaacaagttagtgtaaacctaatggatttctttaagaatatcagtagtgtgggaactggcaatttagtggtcaatttgcttgttggcttgatggaaaaatttgcagacagctcacattcttggaatgttaatcatctgctgcagctctcacgcctgtttcctaaagatgttgtggatgctgtgatagatgtgtactatgtgcttcctcatgctgtaaggctcctgcagggagtacctggtaaaaacatcactgaaggcctcaaggatgtctacagcttcacactccttcatggcataaccatttcaaatatcaccaaggaagacttcgcaattgtgataaaaattcttttggatacaattgaattagtatcagataagccagatattatttcagaggctttagcttgttttcctgtggtttggtgctggaatcacacaaattctggatttcggcagaattcaaagatagacccctgcaatgtccatgggctcatgtcttcttccttttatggcaaagtggccagtatacttgatcatttccacctgtctccccaaggtgaagattcaccatgttcaaatgaaagctcccgaatggaaataactaggaaagtggtctgcataattcatgaattagtggactggaattctattcttctggagctctctgaagtcttccatgttaacatttctcttgtgaaaactgtgcagaaattttggcataagatattaccgtttgtcccaccttcaataaatcaaactagggatagcatctctgaactctgtcctagtggttccataaagcaagttgctttgcaaatcatagaaaaacttaaaaatgtcaactttacaaaagttacatcaggtgaaaatattcttgacaaactaagtagtttaaacaagatccttaacattaatgaagacacagagacatctgttcaaaatattatttcctcaaatttggaaaggacagtacaattgatttctgaagactggagcctagaaaaaagtacgcataatctactctctttattcatgatgctccagaatgcaaatgtcacaggtagcagtttagaagcattatcaagttttattgaaaaaagtgaaacaccttacaactttgaagaactatggcccaagtttcaacaaatcatgaaagacctaacccaagattttagaatcagacacctgctttctgaaatgaacaaaggaatcaaaagtataaattcaatggctcttcaaaagataactttgcagtttgcccatttcctggaaatcctggattcaccgtcattgaagacattagaaattattgaagattttctattggtcacaaaaaactggcttcaggaatatgcaaatgaggattactccagaatgatagaaacattattcattcctgtgaccaatgagagttcaactgaagatatagctttgttagccaaagctattgctactttttggggctctttaaaaaatatatctagagcaggcaattttgatgttgcctttcttacccatctgctaaatcaagaacagctgactaatttctcagttgttcagctgctttttgaaaacatcctaattaatttgatcaataacttagctgggaattctcaggaagcagcttggaacttaaatgatactgaccttcaaataatgaatttcattaaccttatcttgaaccatatgcagtcagaaactagtaggaaaacagttctctctctgagaagcatagtagatttcacagaacagtttttgaaaacattcttctccctttttctaaaggaagattctgagaacaaaatatctcttctgctgaaatatttccacaaagatgttattgcagagatgagttttgtcccaaaagataaaattctagaaattctgaaactggatcaatttcttaccctgatgatacaagacagattgatgaacattttttcaagtttaaaggagactatatatcacctaatgaaaagttcatttatattagacaatggagaattttattttgatactcatcaaggactgaagttcatgcaagatttatttaatgcccttctcagggaaacttcaatgaaaaataagactgaaaataatatagactttttcacagtggtgagtcagttgtttttccatgtgaataagtctgaggacctcttcaaactcaatcaagatcttgggtcagctcttcaccttgtaagagaatgttcaacagagatggcaagacttctggatacaattttacactctcctaataaggacttctatgctttgtatcctaccctccaagaagttatacttgctaatctaacggatttgcttttctttataaataattcattccctctaagaaacagagcaacattagaaattactaagagattagttggtgctatttcaagagcaagtgaagaaagtcacgtcctgaaacccctcttagaaatgtctgggactctggtcatgctgttgaatgacagtgctgacctgagagatcttgccacatcaatggactccattgtgaaacttcttaagctggtcaagaaagtttcggggaagatgtccacagtttttaaaactcattttatctccaataccaaggacagtgtgaaattctttgacactctgtattccatcatgcaacaaagtgttcaaaatcttgtgaaagaaatagctactttaaaaaaaatagatcatttcacatttgaaaagataaatgatttgttggtgccatttcttgacttggcctttgaaatgattggggtagaaccttatatatcatcaaactctgatattttcagtatgtcacctagcatactctcatatatgaaccaatctaaggacttttctgatattttggaagaaattgctgaatttttaacatctgtgaaaatgaacttggaagatatgaggagtcttgcggtagcatttaacaatgagactcaaacattttctatggattctgtcaacttaygggaagaaattctgggttgcttagttcctataaataacatcaccaaccaaatggacttcttataccctaatccaatttccactcatagtggccctcaagatataaaatgggaaataattcatgaagtgatcctttttttggataaaatattatcacaaaacagcacagaaataggatctttcttgaaaatggtgatctgtctcaccttagaagctctttggaaaaacttaaagaaagataattggaatgtttctaatgtgttgatgacgtttactcagcatccaaataaccttttgaaaaccatagaaacagttttagaggcctccagtggaattaaaagtgactatgaaggtgatttgaataaaagtttatattttgacacacctttgagtcagaatataactcatcatcaacttgaaaaagcaatccataatgttttaagtagaatagctctctggaggaaaggacttcgttttaacaactctgaatggataacttccacaagaactttgtttcagccactttttgagattttcattaaagcaaccaccggaaagaatgtcacatcagaaaaagaagagagaaccgagaaagagatgattgactttccttatagtttcaaaccatttttctgtttggagaaatacctgggaggattatttgtattgactaaatactggcaacaaatcccactaacagatcaaagtgttgttgagatttgtgaagttttccagcagactgtgaagccctcagaagccatggagatgctgcagaaagtgaagatgatggtcgtacgtgtgctcaccatcgttgcagaaaacccttcctggaccaaggacattttgtgtgctactctgagttgcaagcaaaatgggataaggcatctcattttatctgctatacaaggggtcactttggcgcaggaccacttccaggaaattgaaaagatatggtcctcgccgaatcagctaaattgtgaaagtcttagcaagaatctttctagcaccttggagagcttcaagagcagcttggaaaatgccactggccaggactgcacaagccagccgaggctggagacggtgcagcagcacttgtacatgttggccaaaagcctcgaggaaacttggtcatcagggaatcccatcatgacttttctcagcaatttcacagtaactgaggatgtaaaaataaaagatttgatgaagaatatcaccaagttgactgaggagcttcgctcttccatccaaatctcgaatgagactatccatagcattctagaagcaaatatttcccactccaaggttctcttcagtgccctcaccgtagctctgtctggaaagtgtgatcaggaaatccttcatctcctgctgacatttcccaaaggggaaaaatcttggatcgcagcggaggaactctgtagcctgccagggtcaaaagtgtattctctgattgtgttgctgagtcgaaacttggatgtgcgagctttcatttacaagactctgatgccttctgaagcaaatggcttgctcaactccttgctggatatagtttccagcctcagcgccttgcttgccaaagcccagcacgtctttgagtatcttcctgagtttcttcacacatttaaaatcactgccttgctagaaaccctggactttcaacaggtttcacaaaatgtccaggccagaagttcagcttttggttctttccagtttgtgatgaagatggtttgcaaggaccaagcatcattccttagcgattctaatatgtttattaatttgcccagagttaaggaactcttggaagatgacaaagaaaaattcaacattcctgaagattcaacaccgttttgcttgaagctttatcaggaaattctacaattgccaaatggtgctttggtgtggaccttcctaaaacccatattgcatggaaaaatactatacacaccaaacactccagaaattaacaaggtcattcaaaaggctaattacaccttttatattgtggacaaactaaaaactttatcagaaacactgctggaaatgtccagccttttccagagaagtggaagtggccagatgttcaaccagctgcaggaggccctgagaaacaaatttgtaagaaactttgtagaaaaccagttgcacattgatgtagacaaacttactgaaaaactccagacatacggagggctgctggatgagatgtttaaccatgcaggcgctggacgcttccgtttcttgggcagcatcttggtcaatctctcttcctgcgtggcactgaaccgtttccaggctctgcagtctgtcgacatcctggagactaaagcacatgaactcttgcagcagaacagcttcttggccagtatcattttcagcaattccttattcgacaagaacttcagatcagagtctgtcaaactgccaccccatgtctcatacacaatccggaccaatgtgttatacagcgtgcgaacagatgtggtaaaaaacccttcttggaagttccaccctcagaatctaccagctgatgggttcaaatataactacgtctttgccccactgcaagacatgatcgaaagagccatcattttggtgcagactgggcaggaagccctggaaccagcagcacagactcaggcggccccttacccctgccataccagcgacctattcctgaacaacgttggtttcttttttccactgataatgatgctgacgtggatggtgtctgtggccagcatggtcagaaagttggtgtatgagcaggagatacagatagaagagtatatgcggatgatgggagtgcatccagtgatccatttcctggcctggttcctggagaacatggctgtgttgaccataagcagtgctactctggccatcgttctgaaaacaagtggcatctttgcacacagcaatacctttattgttttcctctttctcttggattttgggatgtcagtcgtcatgctgagctacctcttgagtgcatttttcagccaagctaatacagcggccctttgtaccagcctggtgtacatgatcagctttctgccctacatagttctattggttctacataaccaattaagttttgttaatcagacatttctgtgccttctttcgacaaccgcctttggacaaggggtattttttattacattcctggaaggacaagagacagggattcaatggaataatatgtaccaggctctggaacaagggggcatgacatttggctgggtttgctggatgattctttttgattcaagcctttattttttgtgtggatggtacttgagcaacttgattcctggaacatttggtttacggaaaccatggtatttcccctttactgcctcatattggaagagtgtgggtttcttggtggagaaaaggcaatactttctaagttctagtctgttcttcttcaatgagaactttgacaataaagggtcatcactgcaaaacagggaaggagagcttgaaggaagtgccccgggagtcaccctggtgtctgtgaccaaggaatatgagggccacaaggctgtggtccaagacctcagcctgaccttctacagagaccaaatcaccgccctgctggggacaaacggtgccgggaaaaccactatcatatccatgttgacggggctccaccctcccacttctggaaccatcatcatcaatggcaagaacctacagacagacctgtcgagggtcagaatggagcttggtgtgtgtccgcagcaggacatcctgttggacaacctcaccgtccgggaacatttgctgctctttgcttccataaaggcgcctcagtggaccaagaaggagctgcatcagcaagtcaatcaaactcttcaggatgtggacttaactcagcatcagcacaaacagacccgagctctgtctggaggcctgaagaggaagctctcccttggcattgctttcatgggcatgtcgaggaccgtggttctggatgagcccaccagtggggtggacccttgctcccggcatagcctgtgggacattctgctcaagtaccgagaaggtcgtacgatcatcttcacaacccaccacctggatgaagccgaagcgctgagtgaccgcgtggccgtcctccagcatgggaggctcaggtgctgcggtcctcccttctgcctgaaggaggcatatggccaggggctccgcctgacactcacgaggcagccttctgttctggaggcccatgatctgaaagacatggcttgtgttacatccctgataaagatctatattccacaagcatttctcaaagacagcagtggaagtgagctgacctacaccattccaaaggacacagacaaggcctgcttgaaagggctcttccaggccctggatgagaacctgcatcagctgcacctgacgggctatgggatctcagacaccaccttagaagaggtgtttttgatgcttttgcaagattccaacaagaaatctcacattgccctggggactgagtcagagctgcagaaccacaggcctacaggacatctgtctggctactgtggctccctagcacggcccgcaactgtgcagggcgtccagctgctccgcgcacaagtggccgcgatcctggcccggaggctccgccgcacgctgcgcgccgggaagagcaccctcgccgacctgctgctgccagtcctcttcgtggccttggccatgggcttgttcatggtgagacccctggccaccgagtaccctcccctcagactcacacctggacattaccagcgggccgagacctactttttcagcagtgggggcgacaacttggacctcacccgtgtgcttctgcggaagtttagagatcaagatttgccctgtgcagatttaaacccacgccagaagaattcttcatgctggcgcacagatcccttttctcacccagaattccaggattcatgtggctgcctgaagtgtccaaatagaagtgctagtgctccctacctgaccaaccacctgggccacacactgttgaatctctcaggcttcaatatggaggagtacttgctggcaccatctgaaaaaccaaggcttggaggttggtcttttggattaaaaatccccagtgaagctggaggtgcaaatggaaacatatcaaaacccccaactctggcaaaggtgtggtataatcagaagggttttcattccctaccttcctacttaaatcatctaaacaaccttattttgtggcagcacctaccccctactgtggactggagacaatacggaataacactctacagccacccatatggaggggccttgctgaacgaggacaagatcctggagagcatccgtcagtgtggagtggccctctgcatcgtgctgggattctccatcctgtctgcatccatcggcagctctgtggtgagggacagggtgattggagccaaaaggttgcagcacataagtggccttggctacaggatgtactggttcacaaacttcctatatgacatgctcttttacttggtttccgtctgcctgtgtgttgccgttattgtcgccttccagttaacagcttttactttccgcaagaacttggcagccacggccctcctgctgtcacttttcggatatgcaactcttccatggatgtacctgatgtccagaatcttttccagttcggacgtggctttcatttcctatgtctcactaaacttcatctttggcctttgtaccatgcccataaccattatgccccggttgctagccatcatctccaaagctaagaatttacagaatatctatgatgtcctcaagtgggtctttactatttttcctcaattctgtcttggtcaaggactggtagaactctgctataatcagatcaaatatgacctgacccacaacttcggcattgattcctatgtgagtccctttgagatgaactttctgggctggatcttcgtgcaactggcctcgcagggcacagtacttctcctcttgagggttctgctacactgggaccttctgcgatggccaaggggtcattctactctccaaggcacagtcaaatcttctaaggatacagatgttgaaaaagaggaaaagagagtgtttgaaggaaggaccaatggagacattcttgtgttatacaaccttagtaaacattatcgacgctttttccagaatattattgctgtgcaagatattagtttgggcataccaaaaggagagtgctttggacttctaggggtgaatggagctgggaagagcacgactttcaaaatgctgaatggtgaagtttctctaacttcaggacatgctatcatcaggactcccatgggagacgccgtggacctgtcttctgctggcacggcaggcgtgctcattggctactgtccccagcaggatgccctggacgagcttctgactggttgggaacatctctattattactgtagcttacgcgggattccaaggcagtgcatccctgaggttgctggagacctcatcaggcgcttacacctcgaagcccacgcggacaaacctgtggccacctacagtgggggaaccaagcggaaactctctacagccctggccctggtggggaaacctgacattcttttattggatgagcccagctctgggatggatccctgctctaagcggtacctgtggcaaacaataatgaaggaggttcgggaaggctgtgctgcggtgctgacctcccacagcatggaggagtgtgaggctctttgcacaagactggccataatggttaacggcagcttcaaatgtcttggttctcctcagcacatcaaaaataggtttggtgatggttatacagtcaaagtttggctctgtaaggaagcaaatcaacattgcactgtttctgaccacttgaagctttattttccaggaattcagttcaagggacagcacctgaatttattagaatatcatgtgccaaaaagatggggatgcctagctgacttgttcaaagttatagagaacaataaaaccttcttgaatattaagcattattccattaaccaaaccactttggagcaggtatttattaattttgcttctgagcagcagcaaactctacaatctactcttgatccatccactgacagtcaccacacacatcacttgcccatctgagcactaaagaagtttccataaggaataaaaccttgtcttccattacaattaacagtcaaggataaaacaagcacgcgcacaatcaaggagctggaacacactctccaggccgtcaaattattctcttgttcattttctattttgaatctccttgttagttaataaccaccaaatggaaaggtcattctttctgcagacttttggggagctcctccaaaacatttgttctctttaccatgccagatggacaccagcttctttgtgacaaaggcatgaatgatttgacagtgtccaaactgagacattctggagctggaaagcctgtcacactagagtgtgtgtgacatgtccactctaaacatgtcacttttctgttaagaaaactgagccccctccccacaggttaaaaaactttagtaacttgtttgtatagaaaatagtaacaaggactattttctattgttgtcatctatttactagatacatgtttttaatgattttaatgtaagcttttattaatactgatgacattatatggtatgatatgaaaaaatcaccaattttttacatataaaagatacctttttaaaaaaataggttttaagagctcttttagtatacactttagcaaaattaattaaattgaactagttactctgtatcaattacagtagttctaccagaatctcccaggttataatttatgagggtagagaaataaaatgtagatgcattttctttttcttcatttggatgaataattactgttttttgttattctaagtcagtgtttttcaaagcgtagtggtccccatattagctgcattgccatcttctgggagcttgcaagaaatgtacattctcaggatccactccagacctattgaatctcaaattctggggcttaaacaagcacattccaagttaagaaccaatgacctaagggaatgtctggttacctcctagttatacaagcaaaatctgcatagtatgtagtcttttttatttatttattttttttttttgaggcggagtctcgctctgtcacccaggctggagtgcagtggcgcgatgtcggctcactgcaagctccgcctcctgggttcacgccattctcctgcctcagcctccccagcagctgggactacaggcacacatcgccacacccggctaatttttagtatttttagtagagacggggtttcaccgtgttagccaggatggtctctatctcctgaccttgtgatccgcccccctccacctcccaaagtgctgggattacaggcgtgagccaccgtgtccggccgtagtttattttaaaatatatttttaaaagctttgtaaaaattatgtcattctcagaattgttgtcttcaaagcattgtcagatgtagagtgctcagatgtggctcttaaagactatatacatctgaatttttcatcctatagttagtaagatgcataaaatcaatccactactgaaatagtttccagtcagacatttctgagttcagacatttctcaacattcttttaacaacatttttctgaatcctcaatagaaaatcacattaatcttattttaaaatttggcctttttcaacactaacgttgagtaccggtagctttgtgatcaaaggcatatacttccttatgagatttctttactaaagcaagatttcattaaatctctatttcctaaatatcattctatacaaaagatattttttaaacggtaaggattaagacaatcactgatagctttgttgtgagcaattttgattcccatgtatcacatgaattacacttccttaaataaattacagtttatggctgtatgatttattctctaattctaacatagtctagttgtcaaaaggaaatatgtaatctttttatgattgttgaatcaataaataccaatttgtgaaacatgaatgtgtttaaactgcagtgaataaatgagatgtgctttaatttaacccaaaaaaaaaaaaaaaaaaaaaaa

SEQ ID NO: 17 - protein, 931 aa, Homo sapiens
MKIQKKLTGCSRLMLLCLSLELLLEAGAGNIHYSVPEETDKGSFVGNIAKDLGLQPQELADGGVRIVSRGRMPLFALNPRSGSLITARRIDREELCAQSMPCLVSFNILVEDKMKLFPVEVEIIDINDNTPQFQLEELEFKMNEITTPGTRVSLPFGQDLDVGMNSLQSYQLSSNPHFSLDVQQGADGPQHPEMVLQSPLDREEEAVHHLILTASDGGEPVRSGTLRIYIQVVDANDNPPAFTQAQYHINVPENVPLGTQLLMVNATDPDEGANGEVTYSFHNVDHRVAQIFRLDSYTGEISNKEPLDFEEYKMYSMEVQAQDGAGLMAKVKVLIKVLDVNDNAPEVTITSVTTAVPENFPPGTIIALISVHDQDSGDNGYTTCFIPGNLPFKLEKLVDNYYRLVTERTLDRELISGYNITITAIDQGTPALSTETHISLLVTDINDNSPVFHQDSYSAYIPENNPRGASIFSVRAHDLDSNENAQITYSLIEDTIQGAPLSAYLSINSDTGVLYALRSFDYEQFRDMQLKVMARDSGDPPLSSNVSLSLFLLDQNDNAPEILYPALPTDGSTGVELAPLSAEPGYLVTKVVAVDRDSGQNAWLSYRLLKASEPGLFSVGLHTGEVRTARALLDRDALKQSLVVAVQDHGQPPLSATVTLTVAVADRISDILADLGSLEPSAKPNDSDLTLYLVVAAAAVSCVFLAFVIVLLAHRLRRWHKSRLLQASGGGLASMPGSHFVGVDGVRAFLQTYSHEVSLTADSRKSHLIFPQPNYADTLISQESCEKKGFLSAPQSLLEDKKEPFSQQAPPNTDWRFSQAQRPGTSGSQNGDDTGTWPNNQFDTEMLQAMILASASEAADGSSTLGGGAGTMGLSARYGPQFTLQHVPDYRQNVYIPGSNATLTNAAGKRDGKAPAGGNGNKKKSGKKEKK

SEQ ID NO: 18 - DNA, 4602 bp, Homo sapiens
atgaagattcagaaaaagctgactggctgcagcaggctgatgcttctgtgtctttctctggagctgctgttggaagctggggctgggaatattcactactcagtgccggaagagacagacaaaggttccttcgtaggcaacatcgccaaggacctagggctgcaaccccaggagctggcagatggcggagtccgcatcgtctccagaggtaggatgccgcttttcgctctgaatcctagaagtggcagcttgatcaccgcgcgcaggatagaccgggaggagctctgcgctcagagcatgccgtgtctcgtgagttttaatatccttgttgaggataaaatgaagctttttcctgttgaagtagaaataattgatattaatgacaacactccccaattccagttagaggaactggagtttaaaatgaatgaaataacgactccaggtaccagagtctcattgccttttgggcaagaccttgatgtgggtatgaactcactccagagctaccaactcagctctaaccctcatttctccctggatgtgcaacagggagccgatgggcctcaacatccagagatggtgctgcagagtcccttagacagagaagaagaagctgtccaccacctcatcctcacagcttctgatgggggtgaaccagtccgttcagggaccctcagaatttacattcaggtggtggatgcaaatgacaatcctccagcatttactcaggcacaataccatataaatgtccccgaaaacgtgccgctgggtactcagctgctcatggtaaatgccactgaccctgatgagggagccaatggggaagtaacgtactcctttcacaatgtagaccacagagtggcccaaatatttcgtttagattcttacacaggagaaatatcaaataaagaaccactagatttcgaagaatacaaaatgtattcaatggaagttcaagcccaggatggtgcggggctcatggctaaagttaaggtactgatcaaagttttggatgtaaatgataatgccccagaagtgaccatcacctctgtcaccactgcagttccagaaaactttcctcctgggaccataattgctcttatcagtgtgcatgaccaggactcaggagacaatggctacaccacatgtttcattcctggaaatttaccctttaaattggaaaagttagttgataattattaccgtttagtgactgaaagaacactggacagagaacttatctctgggtacaacatcacaataacagcaatagaccaaggaactccagctctatctactgaaactcacatttcactactagtgacagatatcaatgacaactccccagtcttccatcaggactcctactctgcctacattcccgaaaacaaccccagaggagcctccatcttctctgtgagggcccacgacttggacagcaatgagaatgcacaaatcacttactccctaatagaggacactatccagggggcacccctatctgcctacctctccatcaactccgacactggggtcctgtatgcgctgcgatccttcgactatgagcagttccgggacatgcaactgaaagtgatggcgcgggacagtggggatccgcccctcagcagcaacgtgtctctcagcctattcctgctggaccagaacgacaacgcgcccgagatcctgtaccccgccctccccacagatggttctaccggcgtggagctggcgcccctctccgcagagcccggctacctggtgaccaaggtggtggcggtggacagagactcgggccagaacgcctggctgtcctaccgcctgctcaaggccagcgagccgggactcttctcggtgggtctgcacacgggcgaggtgcgcacggcgcgagccctgctggacagagacgcgctcaagcagagtctcgtggtggccgtccaggaccacggccagcccccgctctccgccactgtcacgctcaccgtggccgtggccgacaggatctccgacatcctggccgacctgggcagcctcgagccctccgccaaacccaacgattcggacctcactctgtacctggtggtggcggcggccgcggtctcctgcgtcttcctggccttcgtcatcgtgctgctggcgcacaggctgcggcgctggcacaagtcacgtctgctacaggcttcgggaggcggcttagcgagcatgcccggttcgcactttgtgggcgtggacggggttcgggctttcctgcagacctattcccacgaggtctccctcactgcggactcgcggaagagccacctgattttcccccagcccaactatgcggacacactcatcagccaggagagctgtgagaaaaagggttttctatcagcaccccagtctttacttgaagacaaaaaggaaccattttctcagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgccacactgaccaacgcagctggcaagcgggatggcaaggccccagcaggtggcaatggcaacaagaagaagtcgggcaagaaggagaagaagtaacatggaggccaggccaagagccacagggcggcctctccccaaccagcccagcttctccttacctgcacccaggcctcagagtttcagggctaacccccagaatactggtaggggccaaggccatgctccccttgggaaacagaaacaagtgcccagtcagcacctaccccttcccccccagggggttgaatatgcaaaagcagttccgctgggaacccccatccaatcaactgctgtacccatgggggtagtggggttactgtagacaccaagaaccatttgccacaccccgtttagttacagctgaactcctccatcttccaaatcaatcaggcccatccatcccatgcctccctcctccccaccccactccaacagttcctctttcccgagtaaggtggttggggtgttgaagtaccaagtaacctacaagcctcctagttctgaaaagttggaagggcatcatgacctcttggcctctcctttgattctcaatcttcccccaaagcatggtttggtgccagccccttcacctccttccagagcccaagatcaatgctcaagttttggaggacatgatcaccatccccatggtactgatgcttgctggatttagggagggcattttgctaccaagcctcttcccaacgccctggggaccagtcttctgttttgtttttcattgtttgacgtttccactgcatgccttgacttcccccacctcctcctcaaacaagagactccactgcatgttccaagacagtatggggtggtaagataaggaagggaagtgtgtggatgtggatggtgggggcatggacaaagcttgacacatcaagttatcaaggccttggaggaggctctgtatgtcctcaggggactgacaacatcctccagattccagccataaaccaataactaggctggacccttcccactacataatagggctcagcccaggcagccagctttgggctgagctaacaggaccaatggattaaactggcatttcagtccaaggaagctcgaagcaggtttaggaccaggtccccttgagaggtcagaggggcctctgtgggtgctgggtactccagaggtgccactggtggaagggtcagcggagccccagcaggaagggtgggccagccaggccattcttagtccctgggttggggaggcagggagctagggcagggaccaaatgaacagaaagtctcagcccaggatggggcttcttcaacagggcccctgccctcctgaagcctcagtccttcaccttgccaggtgccgtttctcttccgtgaaggccactgcccaggtccccagtgcgccccctagtggccatagcctggttaaagttccccagtgcctccttgtgcatagaccttcttctcccacccccttctgcccctgggtccccggccatccagcggggctgccagagaaccccagacctgcccttacagtagtgtagcgccccctccctctttcggctggtgtagaatagccagtagtgtagtgcggtgtgcttttacgtgatggcgggtgggcagcgggcggcgggctccgcgcagccgtctgtccttgatctgcccgcggcggcccgtgttgtgttttgtgctgtgtccacgcgctaaggcgaccccctcccccgtactgacttctcctataagcgcttctcttcgcatagtcacgtagctcccaccccaccctcttcctgtgtctcacgcaagttttatactctaatatttatatggctttttttcttcgacaaaaaaataataaaacgtttcttctgaaaagctg

SEQ ID NO: 19 - DNA, 3298 bp, Homo sapiens
gcagacgttactgccctcttgcgtgccccggccacccccgggcggcttgtagccggtgcgcggggtggctggggctacgtgcagagctgtcgcggagccggagcagcagcggtgaagcccctcggctcggccgagaccgccgtgcccattgctcgcctcggttgccgccgctttagccgcagccgctgctgccgccgccgggggagaggcagcctattgtctttctccgcggcgaaggtgaggagctgtctcggctcggcccgcgggggagccccgggagccgcacggtggcatttttcaactccgctggagccaatgcccaggaggaacaaagggtgtgctgccagcccctggctcacccagtggcctcgtcccagaaaaagccagaggtagcggccccagccccagagagtgggggtgagtctgtgtttggggagacccaccgggccctgcagggggccatggagaagctgcagcgactttatggaaggagaaggtggacctgaaggagcgggtagagaaactagagcttcaattcatccacctctcaggacagacagacaccatagggagaaagtacatcagccagggggcagtgtcagagacgcagcactgggagaggaggacatcgtcaggctggcccaggaccaggaggagatgaaggtgaacctgcaggagctgcggggcaggtgttgcagcttgtgggagaccacaaggaggggcatggcaaattctgaccattgcccagaaccctgctgatgagcccactctaggagccccaatagcccaggagcttgggtgtgctgacgagcagggtgatcaccactggattgctgacagatagaggacgtgggaccgtgactatcacccctaatctgcagtggatttggctctcggcactcccaggctgggagctggatacctgccctggcagcatgactcagactgcatgacagagaacgtggccagtggagacggcacactggaaatcagagtgaatgttcttgaaagagggtcacgggtcaacaaggcccagccaaaggatgcagtagaaccattttccttagaaatctttgggagtgaagtaggcttcagccactcccatccctgcccttgcggctaccactaccccattagtttagacagggtcgggcggggaggggtgtggagaagaaatgagcttgcctgtggcccccaggctccctctgtcctagctcaggtctgggtgccattctttacactcgtgtgctcgctcacgcacacatcacacaccttgctggtcacacagtcacagactcgcctctgctcctgtggtccagtggccggacaccccctgggatggctcaaaggagtcaggacttggaagtggggacatcagggtagctgaaggaaatccacacacccagagcatctcggagttcagactctcagacctgaagtaggcgcccccgggactgggctaggagttggacggaatggaggatggaggacagcgagaagaaaggaagagaaatgcaacgtgtgggcagccgccaagagtgaaaatagagggaagtgtcatgcaagtgctggacagaaggcggcaggtgggacgagccccacagccccctcctcaaaaacgaccacctccaggactcagtgatccctggggggcaggctctgccagccctcggccacacgtggctccggcacccatggtcccagtgccttggatggagacggccagttctggcggccagatgtggtgctctggaatccagtcccatttccttcctggccacgcctgtccagcggcctcttcagccgcattcagcccctacttacctggggaccccggctggggcacgagagcaccaggggggtagggcccaaagggatcaggggaagcctctggcctggagggtatggggcacgcttccccaagggcggacccggcaggaggaagcccaggagctgggtcctgccgcccaggagctgggccctgccacccaggccgggctagggacatggcagggcctgggcatcctgacgctggacttgggcgacctgggaggcacagggaggggagagatgggcgaccccgccccagcgcagtgccggccacaccccaaggcggttgccagagcttaagccccgcccccagcagcgagaacatcccagctccacaccccccccccccccccccggcagccagtgctccttgtcaagctccccccgtcactccaggtgggagccaccccggtgagggggtgtgccacttgcccccagggcactcctctgggcatcccgggtgggggattttggggccgtggggggcagtctttggtacctgtgttcgtcagggatgctctgacaaccaggtgtcgtccacgggcgggggcatgggcatggtgacagtggtcctgttgatgtcaccgatgatgctgagcgcctccttcagcgcgtggtgcatgtgcagcatctcgtcatgctgctgtgcctgctctgccaactcctccatcagtgtgttctggttcccacatgagtacatattggccagcggctccgagatgatgaactccggggtctgagagtgggcaaacagggaagaaggttgggacctggtgcctgtgccgccctggctgccttgctgggcccttctgggactgtgcgctggacttggagccccttggagtatggcttttcacacgggcttctataccgcttcgactggaagatccacctccccactgccttttctcactcagatggggacaccgaggtccagaggaaaagacacctgtcaaatgtcacagatctgggaggggacttaagacttatcatgccaagaggacacctgtctactcagtttttttttggtggggcggggggcggtgatagggtctcgctctgtcaccaggctggagtacagtgatgactgctcactgcagcctccacctcctgggctcaaagtgatcctccaacgtcagcctctcgagtagctaggactacaggcacatgccaccaccaagcccagctatttttaaaatttttgtgtggagacaaggtctcactatgtggcccaggctggtctcgaactcctgggctcaagtgatcctcctgcctcggcctccaggagtgggagttggagttgatgcctggatacaggagctctgtgggtgggagtgagacaaaacacagggtcctgagctctggggaccaagcaatgtcctctggtgaaaaaaatcctggacttgctggcagaagatttgcctcttactcgccatgtgctctgaatacatttacctgccctctggga

SEQ ID NO: 20 - DNA, 5037 bp, Homo sapiens
gttttcacttggtcggaatggggagagtgtgcaagagatcgctgcgggacaggttcctagagatcgctccgggacggtcgtgacggcccccgagggacatgagagaagaggagcggcgctcaggttattccaggatctttggagacccgaggaaagccgtgttgaccaaaagcaagacaaatgactcacagagaaaaaagatggcagaaccaagggcaactaaagccgtcaggttctgaacagctggtagatgggctggcttactgaaggacatgattcagactgtcccggacccagcagctcatatcaaggaagccttatcagttgtgagtgaggaccagtcgttgtttgagtgtgcctacggaacgccacacctggctaagacagagatgaccgcgtcctcctccagcgactatggacagacttccaagatgagcccacgcgtccctcagcaggattggctgtctcaacccccagccagggtcaccatcaaaatggaatgtaaccctagccaggtgaatggctcaaggaactctcctgatgaatgcagtgtggccaaaggcgggaagatggtgggcagcccagacaccgttgggatgaactacggcagctacatggaggagaagcacatgccacccccaaacatgaccacgaacgagcgcagagttatcgtgccagcagatcctacgctatggagtacagaccatgtgcggcagtggctggagtgggcggtgaaagaatatggccttccagacgtcaacatcttgttattccagaacatcgatgggaaggaactgtgcaagatgaccaaggacgacttccagaggctcacccccagctacaacgccgacatccttctctcacatctccactacctcagagagactcctcttccacatttgacttcagatgatgttgataaagccttacaaaactctccacggttaatgcatgctagaaacacagatttaccatatgagccccccaggagatcagcctggaccggtcacggccaccccacgccccagtcgaaagctgctcaaccatctccttccacagtgcccaaaactgaagaccagcgtcctcagttagatccttatcagattcttggaccaacaagtagccgccttgcaaatccaggcagtggccagatccagctttggcagttcctcctggagctcctgtcggacagctccaactccagctgcatcacctgggaaggcaccaacggggagttcaagatgacggatcccgacgaggtggcccggcgctggggagagcggaagagcaaacccaacatgaactacgataagctcagccgcgccctccgttactactatgacaagaacatcatgaccaaggtccatgggaagcgctacgcctacaagttcgacttccacgggatcgcccaggccctccagccccaccccccggagtcatctctgtacaagtacccctcagacctcccgtacatgggctcctatcacgcccacccacagaagatgaactttgtggcgccccaccctccagccctccccgtgacatcttccagtttttttgctgccccaaacccatactggaattcaccaactgggggtatataccccaacactaggctccccaccagccatatgccttctcatctgggcacttactactaaagacctggcggaggcttttcccatcagcgtgcattcaccagcccatcgccacaaactctatcggagaacatgaatcaaaagtgcctcaagaggaatgaaaaaagctttactggggctggggaaggaagccggggaagagatccaaagactcttgggagggagttactgaagtcttactacagaaatgaggaggatgctaaaaatgtcacgaatatggacatatcatctgtggactgaccttgtaaaagacagtgtatgtagaagcatgaagtcttaaggacaaagtgccaaagaaagtggtcttaagaaatgtataaactttagagtagagtttggaatcccactaatgcaaactgggatgaaactaaagcaatagaaacaacacagttttgacctaacataccgtttataatgccattttaaggaaaactacctgtatttaaaaatagaaacatatcaaaaacaagagaaaagacacgagagagactgtggcccatcaacagacgttgatatgcaactgcatggcatgtgctgttttggttgaaatcaaatacattccgtttgatggacagctgtcagctttctcaaactgtgaagatgacccaaagtttccaactcctttacagtattaccgggactatgaactaaaaggtgggactgaggatgtgtatagagtgagcgtgtgattgtagacagaggggtgaagaaggaggaggaagaggcagagaaggaggagaccagggctgggaaagaaacttctcaagcaatgaagactggactcaggacatttggggactgtgtacaatgagttatggagactcgagggttcatgcagtcagtgttataccaaacccagtgttaggagaaaggacacagcgtaatggagaaaggggaagtagtagaattcagaaacaaaaatgcgcatctctttctttgtttgtcaaatgaaaattttaactggaattgtctgatatttaagagaaacattcaggacctcatcattatgtgggggctttgttctccacagggtcaggtaagagatggccttcttggctgccacaatcagaaatcacgcaggcattttgggtaggcggcctccagttttcctttgagtcgcgaacgctgtgcgtttgtcagaatgaagtatacaagtcaatgtttttccccctttttatataataattatataacttatgcatttatacactacgagttgatctcggccagccaaagacacacgacaaaagagacaatcgatataatgtggccttgaattttaactctgtatgcttaatgtttacaatatgaagttattagttcttagaatgcagaatgtatgtaataaaataagcttggcctagcatggcaaatcagatttatacaggagtctgcatttgcactttttttagtgactaaagttgcttaatgaaaacatgtgctgaatgttgtggattttgtgttataatttactttgtccaggaacttgtgcaagggagagccaaggaaataggatgtttggcacccaaatggcgtcagcctctccaggtccttcttgcctcccctcctgtcttttatttctagccccttttggaacagaaggaccccgggtttcacattggagcctccatatttatgcctggaatggaaagaggcctatgaagctggggttgtcattgagaaattctagttcagcacctggtcacaaatcacccttaattcctgctatgattaaaatacatttgttgaacagtgaacaagctaccactcgtaaggcaaactgtattattactggcaaataaagcgtcatggatagctgcaatttctcactttacagaaacaagggataacgtctagatttgctgcggggtttctctttcaggagctctcactaggtagacagctttagtcctgctacatcagagttacctgggcactgtggcttgggattcactagccctgagcctgatgttgctggctatcccttgaagacaatgtttatttccataatctagagtcagtttccctgggcatcttttctttgaatcacaaatgctgccaaccttggtccaggtgaaggcaactcaaaaggtgaaaatacaaggtgaccgtgcgaaggcgctagccgaaacatcttagctgaataggtttctgaactggcccttttcatagctgtttcagggcctgtttttttcacgttgcagtccttttgctatgattatgtgaagttgccaaacctctgtgctgtggatgttttggcagtgggctttgaagtcggcaggacacgattaccaatgctcctgacaccccgtgtcatttggattagacggagcccaaccatccatcattttgcagcagcctgggaaggcccacaaagtgcccgtatctccttagggaaaataaataaatacaatcatgaaagctggcagttaggctgacccaaactgtgctaatggaaaagatcagtcatttttattttggaatgcaaagtcaagacacacctacattcttcatagaaatacacatttacttggataatcactcagttctctcttcaagactgtctcatgagcaagatcataaaaacaagacatgattatcatattcaattttaacagatgttttccattagatccctcaaccctccacccccagtccaggttattagcaagtcttatgagcaactgggataattttggataacatgataatactgagttccttcaaatacataattcttaaattgtttcaaaatggcattaactctctgttactgttgtaatctaattccaaagccccctccaggtcatattcataattgcatgaaccttttctctctgtttgtccctgtctcttggcttgccctgatgtatactcagactcctgtacaatcttactcctgctggcaagagatttgtcttcttttcttgtcttcaattggctttcgggccttgtatgtggtaaaatcaccaaatcacagtcaagactgtgtttttgttcctagtttgatgcccttatgtcccggaggggttcacaaagtgctttgtcaggactgctgcagttagaaggctcactgcttctcctaagccttctgcacagatgtggcacctgcaacccaggagcaggagccggaggagctgccctctgacagcaggtgcagcagagatggctacagctcaggagctgggaaggtgatggggcacagggaaagcacagatgttctgcagcgccccaaagtgacccattgcctggagaaagagaagaaaatattttttaaaaagctagtttatttagcttctcattaattcattcaaataaagtcgtgaggtgactaattagagaataaaaattactttggactactcaaaaatacaccaaa

SEQ ID NO: 21 - DNA, 3352 bp, Homo sapiens
ggggcgtggcgccggggattgggagggcttcttgcaggctgctgggctggggctaagggctgctcagtttccttcagcggggcactgggaagcgccatggcactgcagggcatctcggtcgtggagctgtccggcctggccccgggcccgttctgtgctatggtcctggctgacttcggggcgcgtgtggtacgcgtggaccggcccggctcccgctacgacgtgagccgcttgggccggggcaagcgctcgctagtgctggacctgaagcagccgcggggagccgccgtgctgcggcgtctgtgcaagcggtcggatgtgctgctggagcccttccgccgcggtgtcatggagaaactccagctgggcccagagattctgcagcgggaaaatccaaggcttatttatgccaggctgagtggatttggccagtcaggaagcttctgccggttagctggccacgatatcaactatttggctttgtcaggtgttctctcaaaaattggcagaagtggtgagaatccgtatgccccgctgaatctcctggctgactttgctggtggtggccttatgtgtgcactgggcattataatggctctttttgaccgcacacgcactggcaagggtcaggtcattgatgcaaatatggtggaaggaacagcatatttaagttcttttctgtggaaaactcagaaattgagtctgtgggaagcacctcgaggacagaacatgttggatggtggagcacctttctatacgacttacaggacagcagatggggaattcatggctgttggagcaatagaaccccagttctacgagctgctgatcaaaggacttggactaaagtctgatgaacttcccaatcagatgagcatggatgattggccagaaatgaagaagaagtttgcagatgtatttgcagagaagacgaaggcagagtggtgtcaaatctttgacggcacagatgcctgtgtgactccggttctgacttttgaggaggttgttcatcatgatcacaacaaggaacggggctcgtttatcaccagtgaggagcaggacgtgagcccccgccctgcacctctgctgttaaacaccccagccatcccttctttcaaaagggatcctttcataggagaacacactgaggagatacttgaagaatttggattcagccgcgaagagatttatcagcttaactcagataaaatcattgaaagtaataaggtaaaagctagtctctaacttccaggcccacggctcaagtgaatttgaatactgcatttacagtgtagagtaacacataacattgtatgcatggaaacatggaggaacagtattacagtgtcctaccactctaatcaagaaaagaattacagactctgattctacagtgatgattgaattctaaaaatggttatcattagggcttttgatttataaaactttgggtacttatactaaattatggtagttattctgccttccagtttgcttgatatatttgttgatattaagattcttgacttatattttgaatgggttctagtgaaaaaggaatgatatattcttgaagacatcgatatacatttatttacactcttgattctacaatgtagaaaatgaggaaatgccacaaattgtatggtgataaaagtcacgtgaaacagagtgattggttgcatccaggccttttgtcttggtgttcatgatctccctctaagcacattccaaactttagcaacagttatcacactttgtaatttgcaaagaaaagtttcacctgtattgaatcagaatgccttcaactgaaaaaaacatatccaaaataatgaggaaatgtgttggctcactacgtagagtccagagggacagtcagttttagggttgcctgtatccagtaactcggggcctgtttccccgtgggtctctgggctgtcagctttcctttctccatgtgtttgatttctcctcaggctggtagcaagttctggatcttatacccaacacacagcaacatccagaaataaagatctcaggaccccccagcaagtcgttttgtgtctccttggactgagttaagttacaagcctttcttatacctgtctttgacaaagaagacgggattgtctttacataaaaccagcctgctcctggagcttccctggactcaacttcctaaaggcatgtgaggaaggggtagattccacaatctaatccgggtgccatcagagtagagggagtagagaatggatgttgggtaggccatcaataaggtccattctgcgcagtatctcaactgccgttcaacaatcgcaagaggaaggtggagcaggtttcttcatcttacagttgagaaaacagagactcagaagggcttcttagttcatgtttcccttagcgcctcagtgattttttcatggtggcttaggccaaaagaaatatctaaccattcaatttataaataattaggtccccaacgaattaaatattatgtcctaccaacttattagctgcttgaaaaatataatacacataaataaaaaaatatatttttcatttctatttcattgttaatcacaactacttactaaggagatgtatgcacctattggacactgtgcaacttctcacctggaatgagattggacactgctgccctcattttctgctccatgttggtgtccatatagtacttgattttttatcagatggcctggaaaacccagtctcacaaaaatatgaaattatcagaaggattatagtgcaatcttatgttgaaagaatgaactacctcactagtagttcacgtgatgtctgacagatgttgagtttcattgtgtttgtgtgttcaaatttttaaatattctgagatactcttgtgaggtcactctaatgccctgggtgccttggcacagttttagaaataccagttgaaaatatttgctcaggaatatgcaactaggaaggggcagaatcagaatttaagctttcatattctagccttcagtcttgttcttcaaccatttttaggaactttcccataaggttatgttttccagcccaggcatggaggatcacttgaggccaagagttcgagaccagcctggggaacttggctggacctccgtttctacgaaataaaaataaaaaaattatccaggtatggtggtgtgtgcctgtagtcctatctactcaagggtggggcaggaggatcacttgagcccaggaatttgaggccacagtgaattaggattgcaccactgcactctagcccaggcaacagaacaagaacctgtctctaaataaataaataaaaataataataataaaaaagatgttttccctacaa

SEQ ID NO: 22 - DNA, 1464 bp, Homo sapiens
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 23 - DNA, 3735 bp, Homo sapiens
agaagaaatagcaagtgccgagaagctggcatcagaaaaacagaggggagatttgtgtggctgcagccgagggagaccaggaagatctgcatggtgggaaggacctgatgatacagaggtgagaaataagaaaggctgctgactttaccatctgaggccacacatctgctgaaatggagataattaacatcactagaaacagcaagatgacaatataatgtctaagtagtgacatgtttttgcacatttccagcccctttaaatatccacacacacaggaagcacaaaaggaagcacagagatccctgggagaaatgcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggattacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaagacccttcgtgttgctgcctaatatgtagctgactgtttttcctaaggagtgttctggcccaggggatctgtgaacaggctgggaagcatctcaagatctttccagggttatacttactagcacacagcatgatcattacggagtgaattatctaatcaacatcatcctcagtgtctttgcccatactgaaattcatttcccacttttgtgcccattctcaagacctcaaaatgtcattccattaatatcacaggattaacttttttttttaacctggaagaattcaatgttacatgcagctatgggaatttaattacatattttgttttccagtgcaaagatgactaagtcctttatccctcccctttgtttgattttttttccagtataaagttaaaatgcttagccttgtactgaggctgtatacagccacagcctctccccatccctccagccttatctgtcatcaccatcaacccctcccatgcacctaaacaaaatctaacttgtaattccttgaacatgtcaggcatacattattccttctgcctgagaagctcttccttgtctcttaaatctagaatgatgtaaagttttgaataagttgactatcttacttcatgcaaagaagggacacatatgagattcatcatcacatgagacagcaaatactaaaagtgtaatttgattataagagtttagataaatatatgaaatgcaagagccacagagggaatgtttatggggcacgtttgtaagcctgggatgtgaagcaaaggcagggaacctcatagtatcttatataatatacttcatttctctatctctatcacaatatccaacaagcttttcacagaattcatgcagtgcaaatccccaaaggtaacctttatccatttcatggtgagtgcgctttagaattttggcaaatcatactggtcacttatctcaactttgagatgtgtttgtccttgtagttaattgaaagaaatagggcactcttgtgagccactttagggttcactcctggcaataaagaatttacaaagagctactcaggaccagttgttaagagctctgtgtgtgtgtgtgtgtgtgtgagtgtacatgccaaagtgtgcctctctctctttgacccattatttcagacttaaaaacaagcatgttttcaaatggcactatgagctgccaatgatgtatcaccaccatatctcattattctccagtaaatgtgataataatgtcatctgttaacataaaaaaagtttgacttcacaaaagcagctggaaatggacaaccacaatatgcataaatctaactcctaccatcagctacacactgcttgacatatattgttagaagcacctcgcatttgtgggttctcttaagcaaaatacttgcattaggtctcagctggggctgtgcatcaggcggtttgagaaatattcaattctcagcagaagccagaatttgaattccctcatcttttaggaatcatttaccaggtttggagaggattcagacagctcaggtgctttcactaatgtctctgaacttctgtccctctttgtgttcatggatagtccaataaataatgttatctttgaactgatgctcataggagagaatataagaactctgagtgatatcaacattagggattcaaagaaatattagatttaagctcacactggtcaaaaggaaccaagatacaaagaactctgagctgtcatcgtccccatctctgtgagccacaaccaacagcaggacccaacgcatgtctgagatccttaaatcaaggaaaccagtgtcatgagttgaattctcctattatggatgctagcttctggccatctctggctctcctcttgacacatattagcttctagcctttgcttccacgacttttatcttttctccaacacatcgcttaccaatcctctctctgctctgttgctttggacttccccacaagaatttcaacgactctcaagtcttttcttccatccccaccactaacctgaatgcctagacccttatttttattaatttccaatagatgctgcctatgggctatattgctttagatgaacattagatatttaaagctcaagaggttcaaaatccaactcattatcttctctttctttcacctccctgctcctctccctatattactgattgcactgaacagcatggtccccaatgtagccatgcaaatgagaaacccagtggctccttgtggtacatgcatgcaagactgctgaagccagaaggatgactgattacgcctcatgggtggaggggaccactcctgggccttcgtgattgtcaggagcaagacctgagatgctccctgccttcagtgtcctctgcatctcccctttctaatgaagatccatagaatttgctacatttgagaattccaattaggaactcacatgttttatctgccctatcaattttttaaacttgctgaaaattaagttttttcaaaatctgtccttgtaaattactttttcttacagtgtcttggcatactatatcaactttgattctttgttacaacttttcttactcttttatcaccaaagtggcttttattctctttattattattattttcttttactactatattacgttgttattattttgttctctatagtatcaatttatttgatttagtttcaatttatttttattgctgacttttaaaataagtgattcggggggtgggagaacaggggagggagagcattaggacaaatacctaatgcatgtgggacttaaaacctagatgatgggttgataggtgcagcaaaccactatggcacacgtatacctgtgtaacaaacctacacattctgcacatgtatcccagaacgtaaag

SEQ ID NO: 24 - protein, 403 aa, Homo sapiens [sequence omitted]

SEQ ID NO: 25 - DNA, 5572 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaagggttttgctacattctaatgcatgtattcgggttaggggaatggagggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaacgacttctccatctcctgtgtaatcaaggccagtgctaaaattcagatgctgttagtacctacatcagtcaacaacttacacttattttactagttttcaatcataatacctgctgtggatgcttcatgtgctgcctgcaagcttcttttttctcattaaatataaaatattttgtaatgctgcacagaaattttcaatttgagattctacagtaagcgttttttttctttgaagatttatgatgcacttattcaatagctgtcagccgttccacccttttgaccttacacattctattacaatgaattttgcagttttgcacattttttaaatgtcattaactgttagggaattttacttgaatactgaatacatataatgtttatattaaaaaggacatttgtgttaaaaaggaaattagagttgcagtaaactttcaatgctgcacacaaaaaaaagacatttgatttttcagtagaaattgtcctacatgtgctttattgatttgctattgaaagaatagggtttttttttttttttttttttttttttttaaatgtgcagtgttgaatcatttcttcatagtgctcccccgagttgggactagggcttcaatttcacttcttaaaaaaaatcatcatatatttgatatgcccagactgcatacgattttaagcggagtacaactactattgtaaagctaatgtgaagatattattaaaaaggtttttttttccagaaatttggtgtcttcaaattataccttcaccttgacatttgaatatccagccattttgtttcttaatggtataaaattccattttcaataacttattggtgctgaaattgttcactagctgtggtctgacctagttaatttacaaatacagattgaataggacctactagagcagcatttatagagtttgatggcaaatagattaggcagaacttcatctaaaatattcttagtaaataatgttgacacgttttccataccttgtcagtttcattcaacaatttttaaatttttaacaaagctcttaggatttacacatttatatttaaacattgatatatagagtattgattgattgctcataagttaaattggtaaagttagagacaactattctaacacctcaccattgaaatttatatgccaccttgtctttcataaaagctgaaaattgttacctaaaatgaaaatcaacttcatgttttgaagatagttataaatattgttctttgttacaatttcgggcaccgcatattaaaacgtaactttattgttccaatatgtaacatggagggccaggtcataaataatgacattataatgggcttttgcactgttattatttttcctttggaatgtgaaggtctgaatgagggttttgattttgaatgtttcaatgtttttgagaagccttgcttacattttatggtgtagtcattggaaatggaaaaatggcattatatatattatatatataaatatatattatacatactctccttactttatttcagttaccatccccatagaatttgacaagaattgctatgactgaaaggttttcgagtcctaattaaaactttatttatggcagtattcataattagcctgaaatgcattctgtaggtaatctctgagtttctggaatattttcttagactttttggatgtgcagcagcttacatgtctgaagttacttgaaggcatcacttttaagaaagcttacagttgggccctgtaccatcccaagtcctttgtagctcctcttgaacatgtttgccatacttttaaaagggtagttgaataaatagcatcaccattctttgctgtggcacaggttataaacttaagtggagtttaccggcagcatcaaatgtttcagctttaaaaaataaaagtagggtacaagtttaatgtttagttctagaaattttgtgcaatatgttcataacgatggctgtggttgccacaaagtgcctcgtttacctttaaatactgttaatgtgtcatgcatgcagatggaaggggtggaactgtgcactaaagtgggggctttaactgtagtatttggcagagttgccttctacctgccagttcaaaagttcaacctgttttcatatagaatatatatactaaaaaatttcagtctgttaaacagccttactctgattcagcctcttcagatactcttgtgctgtgcagcagtggctctgtgtgtaaatgctatgcactgaggatacacaaaaataccaatatgatgtgtacaggataatgcctcatcccaatcagatgtccatttgttattgtgtttgttaacaaccctttatctcttagtgttataaactccacttaaaactgattaaagtctcattcttgtcaaaaaaaaaaaaaaaaaaaaaaaaaaa